(19) United States
(12) Patent Application Publication
(10) Pub. No.: US 2021/0095293 A1
(43) Pub. Date: Apr. 1, 2021

(54) METHODS OF TREATING CANCERS WITH GPR132 INHIBITORS

(71) Applicant: The Board of Regents of the University of Texas System, Austin, TX (US)

(72) Inventors: Wing Yin CHENG, Dallas, TX (US); Yihong WAN, Dallas, TX (US); Peiwen CHEN, Dallas, TX (US)

(21) Appl. No.: 16/098,781

(22) PCT Filed: May 5, 2017
(86) PCT No.: PCT/US2017/031270

§ 371 Date: Nov. 2, 2018

Related U.S. Application Data
(60) Provisional application No. 62/407,627, filed on Oct. 13, 2016.
(60) Provisional application No. 62/332,262, filed on May 5, 2016.

(51) Int. Cl. C12N 15/113; C12Q 1/6886; A61K 31/4439; G01N 33/574

(52) U.S. Cl. CPC C12N 15/1138; C12Q 1/6886; A61K 31/4439; G01N 2333/705; C12N 2310/14; C12Q 2600/112; C12Q 2600/158; G01N 33/57415

(57) ABSTRACT

The current disclosure describes therapeutic methods for treating cancer by inhibition of G protein-coupled receptor 132 (GPR132) in the tumor microenvironment. Aspects of the disclosure relate to a method for reducing and/or inhibiting cancer cell proliferation or tumor growth in a subject having cancerous cells or tumor growth, the method comprising administering an effective amount of an agent that inhibits GPR132 to the subject.

This application claims priority to U.S. Provisional Patent Application Ser. Nos. 62/332,262 filed May 5, 2016 and 62/407,627 filed Oct. 13, 2016, each of which is incorporated herein by reference in its entirety.

### BACKGROUND OF THE INVENTION

### I. Field of the Invention

Embodiments are directed generally to biology and medicine. In certain aspects there are methods and compositions for treating cancers.

### II. Background

How immune cells in the tumor microenvironment modulate cancer malignancy is a fundamental and fascinating question with tremendous therapeutic significance for cancer intervention. Traditional studies target the cancer cells themselves, but the idea of targeting pro-cancer aspects of the tumor microenvironment is somewhat new. Tumor-associated macrophage (TAM) have been implicated in playing a role in cancer progression. Although numerous clinical studies and experimental mouse models support that macrophages generally play a pro-cancer role, anti-tumor properties have also been reported for certain subtypes of macrophages, suggesting that macrophage regulation of cancer malignancy is pleotropic and context-dependent (Krzeszinski and Wan, 2015; Noy and Pollard, 2014; Qian and Pollard, 2010; Ruffell et al., 2012; Ruffell and Coussens, 2015).

There is a need in the art for a better understanding of TAMs and how they contribute to tumor progression. Such advancements could lead to therapies that reduce the ability of cancer cells to proliferate and grow by manipulating the tumor microenvironment.

### SUMMARY OF THE INVENTION

The current disclosure fulfills the aforementioned need in the art by providing therapeutics that treat cancer by inhibition of G protein-coupled receptor 132 (GPR132) in the tumor microenvironment. Accordingly, aspects of the disclosure relate to a method for reducing and/or inhibiting cancer cell proliferation or tumor growth in a subject having cancerous cells or tumor growth, the method comprising administering an effective amount of an agent that inhibits GPR132 to the subject. Further aspects relate to a method for inhibiting or reducing cancer metastasis, the method comprising administering an effective amount of an agent that inhibits GPR132 to the subject. Further aspects relate to a method for reducing inflammation in a tumor microenvironment or a subject having a tumor comprising administering an effective amount of an agent that inhibits GPR132 to the subject. A further aspect relates to a method for treating an inflammatory disorder in a subject in need thereof comprising administering an effective amount of an agent that inhibits GPR132 to the subject. In some embodiments, the inflammatory disorder is associated with a cancerous condition. In some embodiments, the inflammatory disorder is associated with an autoimmune condition. In some embodiments, the autoimmune or cancerous condition is one described herein.

In some embodiments, the tumor or cancer cell has no detectable expression of GPR132. In some embodiments, the tumor or cancer cell has not statistically significant detectable expression of GPR132. In some embodiments, the tumor or cancer cell has a level of GPR132 expression that is not significantly (or statistically) different than the level of GPR132 in a non-cancerous cell. In some embodiments, the tumor or cancer cell has a level of GPR132 expression that is the same or less than the level of expression in a non-cancerous control cell. In some embodiments, the tumor or cancer cell and the non-cancerous control cell are the same cell type.

The cell may be any appropriate cell type. In some embodiments, GPR132 is inhibited in an immune cell. In some embodiments, the immune cell is a macrophage. Immune cells include, for example, B Cells; dendritic cells; granulocytes; innate lymphoid cells (ILCs); megakaryocytes; monocytes/macrophages; natural killer (NK) cells; platelets; red blood cells (RBCs); T Cells such as killer T cells, helper T cells, gamma delta T cells, regulatory T cells; phagocytes; neutrophils; mast cells; eosinophils; basophils; and thymocytes. In some embodiments, the immune cell is a macrophage.

The agent may be any appropriate agent that inhibits GPR132. In some embodiments, the agent is a nucleic acid inhibitor, an antibody inhibitor, a peptide or polypeptide inhibitor, or a small molecule inhibitor. In some embodiments, the agent inhibits a GPR132 polypeptide. In some embodiments, the agent inhibits a GPR132 nucleic acid expression.

The agent may be administered in any appropriate manner. In some embodiments, the agent is administered systemically. In some embodiments, the administration is parenteral. In some embodiments, the administration is intravenous or intratumoral. In some embodiments, the administration is a route of administration described herein.

In some embodiments of the methods of the disclosure, the tumor microenvironment comprises macrophages. In some embodiments, the macrophages express GPR132. In some embodiments, the cancer cell or tumor is a breast cancer cell or a tumor in the breast. In some embodiments, the breast cancer comprises ductal carcinoma in situ, invasive ductal carcinoma, tubular carcinoma of the breast, medularry carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, paget's disease of the nipple, phyllodes tumors of the breast, luminal breast cancer, and/or recurrent and metatastatic breast cancer.

In some embodiments of the methods described herein, the subject is a human subject. The term “subject,” “individual” or “patient” is used interchangeably herein. In some embodiments, the subject has been diagnosed with a cancer or inflammatory disorder. In some embodiments, the subject has previously been treated for the cancer or inflammatory disorder. In some embodiments, the method comprises administration of a second therapeutic agent. In some embodiments, the second therapeutic agent is one described herein.

Further aspects relate to a method for classifying a cancer patient, comprising: measuring the level of expression of GPR132 in a cancer sample of the patient; classifying the patient as having a favorable prognosis based on a lower expression of GPR132 in the sample as compared to a control or reference level that is normal or indicating favorable prognosis, or classifying the patient as having a poor prognosis based on a higher expression level of GPR132 as compared to the control or a reference level.

In some embodiments, the method further comprises obtaining the cancer sample. In some embodiments, the cancer sample is provided. In some embodiments, measuring the level of GPR132 expression comprises assaying nucleic acids in the cancer sample. In some embodiments, assaying nucleic acids comprises using PCR, microarray analysis, digital PCR, dd PCR (digital droplet PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), RT-PCR, in situ hybridization, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco), next generation RNA sequencing, or a combination thereof. In some embodiments, measuring the level of GPR132 expression comprises measuring protein expression in the cancer sample. In some embodiments, measuring protein expression comprises performing ELISA, MA, FACS, dot blot, Western Blot, immunohistochemistry, antibody-based radioimaging, mass spectroscopy, or a combination thereof.

In some embodiments, the cancer is breast cancer. In some embodiments, the cancer is one described herein. In some embodiments, the cancer is pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, or wilms tumor (kidney cancer).

In some embodiments, the method further comprises monitoring the patient for cancer under intensive surveillance after the patient has been classified as having a poor prognosis. In some embodiments, the method further comprises monitoring the patient for cancer under regular surveillance after the patient has been classified as having a favorable prognosis. In some embodiments, the method further comprises treating the patient for cancer with a pharmaceutical composting comprising a GPR132 inhibitor and/or rosiglitazone after the patient has been classified as having a poor prognosis. In some embodiments, the method further comprises treating the patient for cancer under regular surveillance if the patient is classified as having a favorable prognosis.

Any of the methods described herein may be implemented on tangible computer-readable medium comprising computer-readable code that, when executed by a computer, causes the computer to perform one or more operations. In some embodiments, there is a tangible computer-readable medium comprising computer-readable code that, when executed by a computer, causes the computer to perform operations comprising: a) receiving information corresponding to the level of expression of GPR132 in a cancer sample of a patient suspected of having or determined to have a cancer; and b) determining a difference value in the expression level of GPR132 using the information corresponding to the level of expression of GPR132 in the cancer sample as compared to a control or reference level that is normal or indicating favorable prognosis. In further embodiments, the receiving information comprises receiving the information corresponding to the expression level from a tangible data storage device.

In some embodiments, the method further comprises recording the classification or the expression level of GPR132 in a tangible, computer-readable medium or a tangible data storage device. In some embodiments, the method further comprises reporting the classification or the expression level of GPR132 to the patient, a health care payer, a physician, an insurance agent, or a tangible data storage device. In additional embodiments the medium further comprises computer-readable code that, when executed by a computer, causes the computer to perform one or more additional operations comprising sending information corresponding to the difference value to a tangible data storage device; calculating a prognosis score for the patient; classifying the patient as having a favorable prognosis or poor prognosis; or determining a management, surveillance or treatment plan for the patient.

In some embodiments, the prognosis score is expressed as a number that represents a probability of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% likelihood (or any range derivable therein) that a patient has a chance of poor survival or cancer recurrence or poor response to a particular treatment. Alternatively, the probability may be expressed generally in percentiles, quartiles, or deciles.

In some embodiments, determination of calculation of a diagnostic, prognostic, or risk score is performed by applying classification algorithms based on the expression values of biomarkers with differential expression p values of about, between about, or at most about 0.005, 0.006, 0.007, 0.008, 0.009, 0.01, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.1, 0.2, 0.3, 0.4, 0.5, 0.6, 0.7, 0.8, 0.9 or higher (or any range derivable therein). In certain embodiments, the prognosis score is calculated using one or more statistically significantly differentially expressed biomarkers (either individually or as difference pairs), including expression or activity levels in a gene or protein corresponding to GPR132.

Embodiments concern determining that the level of expression or activity of GPR132. In some embodiments, that level is compared to a control in order to determine whether the expression level or activity of GPR132 is elevated as compared to the level in non-cancerous tissue. The control may be a non-cancerous tissue or it may be a cancerous tissue. If the control is a cancerous tissue a sample may be determined to have an elevated level of GPR132 because the levels in the control and the patient sample are similar, such as within, at least or at most 1, 2, 3, or 4 standard deviations (or any range derivable therein) of one another.

In some embodiments, the biological sample, as stated herein, comprises a hematopoietic cell or a cell progeny thereof. In some embodiments, the biological sample from the patient is a sample from a primary cancer tumor. In some embodiments, the biological sample is from a tissue or organ as described herein. Non-limiting examples of the sample include a tissue sample, a whole blood sample, a urine sample, a saliva sample, a serum sample or a fecal sample. In some embodiments, the biological sample comprises immune cells. In some embodiments, the immune cells are macrophages. In some embodiments the immune cells or macrophages are isolated.

In some embodiments, the method further comprises excluding the tumor cells from the biological sample. In some embodiments, the biological sample excludes tumor cells. In some embodiments, the expression of GPR132 is measured in a hematopoietic cell or a cell progeny thereof.

A further aspect relates to a method for treating a patient for aggressive or non-aggressive cancer comprising: treating the patient for aggressive cancer after the patient is determined to have an elevated level of GPR132 expression in a biological sample from the patient compared to a biological sample from a patient with non-aggressive cancer; or treating the patient for non-aggressive cancer after the patient is determined to have a GPR132 level of expression that is lower than or not significantly different than a level of GPR132 expression in a biological sample from a patient with non-aggressive cancer. In some embodiments, the method further comprises measuring the expression or activity level of GPR132 in a biological sample from the patient. In some embodiments, the method further comprises comparing the expression or activity level of GPR132 in the biological sample from the patient to the expression or activity level of GPR132 in a biological sample from a patient with non-aggressive cancer. In some embodiments, the method further comprises comparing the expression or activity level of GPR132 in the biological sample from the patient to a cut-off value. In some embodiments, the aggressive cancer comprises breast cancer. In some embodiments, the aggressive cancer comprises distant metastasis. In some embodiments, the distant metastasis is liver metastasis or bone metastasis. In some embodiments, the biological sample comprises a hematopoietic cell or a cell progeny thereof. In some embodiments, the biological sample comprises macrophages. In some embodiments, the macrophages are isolated. In some embodiments, the method further comprises excluding the tumor cells from the biological sample. In some embodiments, the biological sample excludes tumor cells. In some embodiments, the expression of GPR132 in a hematopoietic cell or a cell progeny thereof is measured. In some embodiments, the treatment for non-aggressive cancer comprises surgical incision of the primary tumor. In some embodiments, the treatment for non-aggressive cancer excludes chemotherapy. In some embodiments, the treatment for the aggressive cancer comprises a GRP132 inhibitor and/or rosiglitazone. In some embodiments, the treatment for the aggressive cancer comprises surgical removal of one or more secondary tumors. In some embodiments, the secondary tumor is a distant liver or bone metastasis. In some embodiments, the non-aggressive cancer treatment excludes surgical removal of one or more secondary tumors.

Further aspects relate to a kit comprising an agent for detecting GPR132 expression. In some embodiments, the agent detects GPR132 protein expression or mRNA expression. In some embodiments, the agent comprises one or more nucleic acid probes for amplification of a GPR132 nucleic acid from a biological sample. In some embodiments, the agent is an antibody. In some embodiments, the agent is labeled.

Further aspects relate to a method for diagnosing a patient with aggressive or non-aggressive cancer comprising: diagnosing the patient as having or likely to have aggressive cancer or providing an analysis or report that the patient has or likely has aggressive cancer when the expression or activity level of GPR132 in a biological sample from the patient is determined to be elevated compared to the expression or activity level of GPR132 in a biological sample from a patient with non-aggressive cancer; or diagnosing the patient as having or likely to have non-aggressive cancer or providing an analysis or report that the patient has or likely has non-aggressive cancer when the expression or activity level of GPR132 in the biological sample from the patient is determined to be not significantly different or lower than the expression or activity level of GPR132 in a biological sample from a patient with non-aggressive cancer. In some embodiments, the method further comprises measuring the expression or activity level of GPR132 in a biological sample from the patient. In some embodiments, the biological sample comprises a hematopoietic cell or a cell progeny thereof. In some embodiments, the biological sample comprises macrophages. In some embodiments, the macrophages are isolated. In some embodiments, the method further comprises excluding the tumor cells from the biological sample. In some embodiments, the biological sample excludes tumor cells. In some embodiments, the expression of GPR132 is measured in a hematopoietic cell or a cell progeny thereof. In some embodiments, the method further comprises comparing the expression or activity level of GPR132 in the biological sample from the patient to a control level of expression. In some embodiments, the aggressive cancer comprises distant metastasis. In some embodiments, the distant metastasis is liver or bone metastasis. In some embodiments, the biological sample from the patient is a sample from a primary cancer tumor. In some embodiments, the method further comprises comparing the level of expression of GPR132 in the biological sample from the patient to a cut-off value. In some embodiments, the method further comprises treating the patient for aggressive or non-aggressive cancer. In some embodiments, the treatment for non-aggressive cancer comprises surgical incision of the primary tumor. In some embodiments, the treatment for non-aggressive cancer excludes chemotherapy. In some embodiments, the treatment for the aggressive cancer comprises a GRP132 inhibitor and/or rosiglitazone. In some embodiments, the treatment for the aggressive cancer comprises surgical removal of one or more secondary tumors. In some embodiments, the secondary tumor is a distant liver or bone metastasis. In some embodiments, the non-aggressive cancer treatment excludes surgical removal of one or more secondary tumors.

In some embodiments, the elevated level is at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900%, or any derivable range therein.

The terms “ameliorating,” “inhibiting,” or “reducing,” or any variation of these terms, when used in the claims and/or the specification includes any measurable decrease or complete inhibition to achieve a desired result.

As used herein, “increased expression” or “decreased expression” refers to an expression level of a biomarker in the subject's sample as compared to a reference level representing the same biomarker or a different biomarker. In certain aspects, the reference level may be a reference level of expression from a normal sample, such as a non-cancerous tissue from the same subject, particularly normal mucosa, or a sample from a different subject that does not have the cancer to be treated. Alternatively, the reference level may be a reference level of expression from a different subject or group of subjects, such as a reference level of expression from a subject or a group of subjects that have a favorable prognosis of cancer, such as having at most 20, 30, 40, or 50, 60, 70, 80% recurrence risk (or any range derivable therefrom) or at least 50, 60, 70, 80, or 90% survival chance (or any range derivable therefrom) of cancer relative to a group of poor prognosis or favorable prognosis subjects or a combination thereof. Alternatively, the reference level may be a reference level of expression from a subject or a group of subjects that has a poor prognosis, such as having a high recurrence risk of more than 50, 60, 70, 80, or 90 (or any range derivable therefrom) or at most 20, 30, 40, or 50, 60, 70, 80% survival chance (or any range derivable therefrom) relative to a group of poor prognosis or favorable prognosis subjects or a combination thereof. The combined group may be randomly selected or may be a group of clinical trial subjects, subjects in a particular geographic area, an age group, a gender group, or a stage of colorectal cancer, or any group based on one or more predetermined classification criteria, like inclusion or exclusion of patients that have favorable or poor prognosis.

A person of ordinary skill in the art understands that an expression level from a test subject may be determined to have an elevated level of expression, a similar level of expression or a decreased level of expression compared to a reference level.

“Diagnosis” may refer to the process of attempting to determine or identify a possible disease or disorder, or to the opinion reached by this process. From the point of view of statistics the diagnostic procedure may involve classification tests.

“Prognosis” may refer to a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. Prognosis may also include prediction of favorable responses to cancer treatments, such as a conventional cancer therapy.

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets. In some embodiments, the subject is a human subject.

In some embodiments, the method further comprises administration of pain medication and/or antibiotics. In some embodiments, the method further comprises administration of a traditional therapeutic for cancer such as chemotherapy and/or surgery. In some embodiments, the additional therapeutic is one known in the art and/or described herein.

Use of the one or more compositions may be employed based on methods described herein. Use of one or more compositions may be employed in the preparation of medicaments for treatments according to the methods described herein. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

As used herein, “treatment” or “therapy” is an approach for obtaining beneficial or desired clinical results. This includes: reduce the alleviation of symptoms, the reduction of inflammation, the inhibition of cancer cell growth, and/or the reduction of tumor size. In some embodiments, the term treatment refers to the inhibition or reduction of cancer cell proliferation in a subject having cancer.

The term “therapeutically effective amount” refers to an amount of the drug that treats or inhibits cancer in the subject. In some embodiments, the therapeutically effective amount inhibits at least or at most or exactly 100, 99, 98, 96, 94, 92, 90, 85, 80, 75, 70, 65, 60, 55, 50, 40, 30, 20, or 10%, or any derivable range therein, of GPR132 activity.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used in this specification and the claim(s), when referring to a particular therapeutic drug regimen, the words “consisting essentially of” includes therapeutic drug remiments including, as active ingredients, only the recited active ingredients and excludes any active ingredients not recited.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1H. Macrophage PPARγ deletion enhances mammary tumor growth in vivo. (FIG. 1A) Tie2Cre-induced mf-g-KO mice (n=26; second bar of each time-point of days after E0771 cell fat pad injection) showed enhanced tumor growth compared to control mice (n=16; first bar of each time-point of days after E0771 cell fat pad injection) as indicated by earlier onset and larger tumor volume. EO771 mouse mammary tumor cells were injected into the mammary fat pad of 6-8 weeks old female mice. (FIG. 1B) LyzCre-induced mf-g-KO mice (n=6; second bar of each time-point of days after E0771 cell fat pad injection) showed augmented tumor growth compared to control mice (n=6; first bar of each time-point of days after E0771 cell fat pad injection) as indicated by earlier onset and larger tumor volume. (FIGS. 1C-1D) Quantification of cell proliferation markers Ki67 (FIG. 1C) and phosphor histone H3 (PH3) (FIG. 1D) in tumor sections showed increased cell proliferation in mf-g-KO mice (n=4). (FIGS. 1E-1G) RT-QPCR analyses showed an increased expression of pro-inflammatory genes in tumor tissues (FIG. 1E), bone marrow (BM) (FIG. 1F) and spleen (FIG. 1G) from mf-g-KO mice (n=3). (FIG. 1H) Immunofluorescence staining of tumor sections for macrophage marker CD11b showed an enhanced macrophage recruitment in the tumors from both Tie2Cre- and LyzCre-induced mf-g-KO mice compared with control mice (n=4). Error bars, SD; *, p<0.05; **, p<0.01; ***, p<0.005; ****, p<0.001; n. s. non-significant.

FIGS. 2A-2J. Macrophage PPARγ deletion exacerbates breast cancer cell proliferation and attenuates the anti-tumor effect of rosiglitazone. (FIG. 2A) A diagram of mouse macrophage and human breast cancer cell co-culture. Progenitors in bone marrow or spleen were differentiated into macrophages with M-CSF for 9 days before the seeding of luciferase-labelled 1833 human breast cancer cells to the cultures. For rosiglitazone (Rosi) pre-treatment, macrophages were treated with 1 μM Rosi or vehicle control for the last 24 hrs of macrophage differentiation; after medium were removed and cells were washed, cancer cells were added to the macrophage cultures in fresh medium without Rosi or vehicle. (FIG. 2B) Cancer cell proliferation was increased when co-cultured with PPARγ-deficient macrophages derived from bone marrow (left) or spleen (right) of mf-g-KO mice compared with WT control macrophages (n=3). Cancer cell growth was quantified by luciferase signal for 2-6 days. (FIG. 2C) PPARγ-deficient macrophages promoted tumor cell colony formation in the co-cultures (n=3). Tumor cells were cultured for 11-12 days for the colonies to form. Left, representative images of crystal violet staining. Right, quantification of colony formation. The first bar of each group is the control, and the second bar of each group is mf-g-KO. (FIGS. 2D-2E) Co-culture with PPARγ-deficient macrophages resulted in higher expression of proliferation markers (FIG. 2D) and lower expression of apoptosis markers (FIG. 2E) in breast cancer cells (n=3). Human gene expression in cancer cells was quantified by RT-QPCR and human-specific primers. (FIG. 2F) PPARγ-deficient macrophages exhibited higher expression of pro-inflammatory genes (n=3). BMMf, bone marrow macrophage; SpMf, spleen macrophage. The first bar of each group is the control, and the second bar of each group is mf-g-KO. (FIG. 2G) PPARγ-deficient macrophages displayed higher levels of anti-apoptotic genes (left) and lower levels of pro-apoptotic genes (right) (n=3). The first bar of each group is the control, and the second bar of each group is mf-g-KO. (FIG. 2H) PPARγ-deficient macrophages showed increased proliferation (n=3). The number of metabolically active cells was determined by ATP content using the CellTiter-Glo® Assay. The first bar of each group is the control, and the second bar of each group is mf-g-KO. (FIG. 2I) Co-culture with Rosi pre-treated macrophages inhibited breast cancer cell growth compared with vehicle (Veh) pre-treated macrophages in a macrophage-PPARγ-dependent manner (n=3). The bars of each group are, from left to right, 1) Ctrl+Veh, 2) Ctrl+Rosi, 3) mf-g-KO+Veh, and 4) mf-G-KO+Rose. (FIG. 2J) The ability of Rosi to suppress tumor growth in vivo was significantly attenuated in mf-g-KO mice (n=6). Four days after EO771 cell mammary fat pad injection, mf-g-KO mice or control mice were treated with Veh or Rosi (10 mg/kg) every two days for one week before tumor volume measurement. Error bars, SD; *, p<0.05; **, p<0.01; ***, p<0.005; ****, p<0.001; n. s. non-significant.

FIGS. 3A-3J. PPARγ represses Gpr132 transcription in macrophages. (FIGS. 3A-3B) Physical contact is required for the pro-tumor effects of PPARγ-deficient macrophages. (FIG. 3A) A schematic diagram of the co-culture vs. trans-well systems. (FIG. 3B) Tumor cell proliferation was enhanced by co-culture with PPARγ-deficient macrophages but not by their conditioned medium delivered via trans-well (n=3). The bars of each group are, from left to right, 1) Ctrl BMW, 2) g-KO BMMf, 3) Ctrl SpMf, and 4) g-KO SpMf. FIG. 3C) Gpr132 was predominantly expressed in the hematopoietic cell types and tissues (n=3). (FIG. 3D) Gpr132 was expressed in macrophages but largely absent in breast cancer cells. mBMMf, mouse bone marrow macrophage; mSpmf, mouse spleen macrophage; mBC, mouse breast cancer cells; hBC, human breast cancer cells. (FIG. 3E) Gpr132 mRNA levels were significantly higher in PPARγ-deficient macrophages compared with control macrophages, either in macrophage cultures alone or in macrophages co-cultured with human breast cancer cells (n=3). The first bar of each group is the control, and the second bar of each group is mf-g-KO. (FIG. 3F) Gpr132 protein expression was significantly higher in PPARγ-deficient macrophages (n=3). (FIG. 3G) PPARγ activation by rosiglitazone reduced Gpr132 mRNA in WT macrophages but not in PPARγ-deficient macrophages (n=3). The first bar of each group is the vehicle control, and the second bar of each group is Rosi. (FIG. 3H) Transcriptional output from both 0.5 Kb and 1 Kb Gpr132 promoters was reduced by the co-transfection of PPARγ and further diminished by rosiglitazone (n=3). HEK293 cells were transfected with PPARγ and its heterodimer partner retinoic X receptor α (RXRα), together with a luciferase reporter driven by 0.5 Kb or 1 Kb Gpr132 promoter, and compared with vector-transfected controls. Next day, cells were treated with rosiglitazone or vehicle control for 24 hours before harvest and reporter analyses. The first bar of each group is the vehicle control, and the second bar of each group is Rosi. (FIG. 3I) ChIP assay of PPARγ binding to the endogenous Gpr132 promoter in macrophages. PPRE region in the Gpr132 promoter was pull-down with anti-PPARγ antibody (second bar of each group) or an IgG control antibody (first bar in each group) in RAW264.7 mouse macrophages and detected by QPCR (n=3). An upstream Gpr132 promoter region served as a negative control. (FIG. 3J) ChIP assay of H3K9Ac active transcription histone mark at the Gpr132 transcription start site showed that rosiglitazone represses the transcriptional activity from Gpr132 promoter (n=3). RAW264.7 macrophages were treated with 1 μM rosi or vehicle control for 4 hours before harvest. Error bars, SD; *, p<0.05; **, p<0.01; ***, p<0.005; ****, p<0.001; n. s. non-significant.

FIGS. 4A-4F. Gpr132 is repressed by PPARγ in human macrophages and correlates with human breast cancer. (FIG. 4A) Human Gpr132 expression in hPBMN-derived macrophages was blunted by rosiglitazone treatment (n=3). Macrophages were treated with 1 μM rosiglitazone or vehicle for 4 hours. (FIG. 4B) TCGA BRCA data analysis showed that compared with normal breast samples, breast cancer lesions displayed higher Gpr132 expression. Normal Breast (n=111); Infiltrating Ductal Carcinoma (n=750); Infiltrating Lobular Carcinoma (n=168); Medullary Carcinoma (n=5); Mixed Histology (n=29); Mucinous Carcinoma (n=14); Other Histology (n=44). Error bars, SE. (FIG. 4C) TCGA BRCA data analysis showed that compared with ER+breast cancers (n=746), ER-breast cancers (n=221) exhibited higher Gpr132 expression. Error bars, SE. (FIGS. 4D-4E) Immunohistochemistry (IHC) of human tissue microarrays showed higher Gpr132 expression in breast cancer tissues (n=16) compared with normal breast tissues (n=13). Tissues were stained with anti-Gpr132 and hematoxylin. (FIG. 4D) Representative images. Scale bars, 200 μm. (FIG. 4E) Quantification of relative IHC scores. Error bars, SE. (FIG. 4F) Linear regression analyses of TCGA BRCA data showed that Gpr132 expression was positively correlated with the expression of CCL2 (MCP-1), MMP9 and PTGS2 (COX-2) in breast cancer lesions (n=805). Error bars, SD (FIG. 4A) or SE (FIGS. 4B, 4C, 4E); *, p<0.05; **, p<0.01; ***, p<0.005; ****, p<0.001; n. s. non-significant.

FIGS. 5A-5Q. Macrophage Gpr132 promotes tumor growth and mediates the anti-tumor effect of rosiglitazone (FIGS. 5A-5B) Gpr132 knockdown (second bar of each group) decreased Gpr132 mRNA (FIG. 5A) and protein (FIG. 5B) in macrophages (n=3). (FIG. 5C) In co-cultures, Gpr132 knockdown in macrophages reduced cancer cell growth (n=3). (FIGS. 5D-5E) Gpr132 over-expression (second bar in (FIG. 5D)) increased both mRNA (FIG. 5D) and protein (FIG. 5E) in macrophages (n=3). (FIG. 5F) In co-cultures, Gpr132 over-expression in macrophages enhanced cancer cell growth (n=3). Cancer cell alone without macrophages (no mf) served as a negative control. (FIG. 5G) Gpr132-KO macrophages (second bar of each group) exhibited lower expression of pro-inflammatory genes compared with WT controls (n=3; first bar of each group). (FIG. 5H) Gpr132-KO macrophages displayed higher levels of pro-apoptotic genes and lower levels of anti-apoptotic genes (n=3). The 4 bars in each group from left to right correspond to 1) Gpr132-WT BMMf, 2) Gpr132-KO BMMf, 3) Gpr132-WT SpMf, and 4) Gpr132-KO SpMf. (FIGS. 5I-5J) In vitro co-cultures showed that Gpr132 deletion in macrophages significantly reduced the ability of macrophages to promote cancer cell colony formation (FIG. 5I) and proliferation (FIG. 5J) (n=3). (FIG. 5K) In vivo mammary fat pad tumor grafts showed that tumor growth was significantly diminished in Gpr132-KO mice (third bar of each group) compared with WT (first bar of each group) or Gpr132-Het mice (second bar of each group) (n=6). (FIG. 5L) In in vitro co-cultures, Rosi pre-treated WT macrophages but not Rosi pre-treated Gpr132-KO macrophages was able to inhibit cancer cell growth (n=3). (FIG. 5M) The ability of Rosi to suppress tumor growth in vivo was abolished in Gpr132-KO mice (n=6). Four days after EO771 cell mammary fat pad injection, Gpr132-KO or WT mice were treated with Veh or Rosi (10 mg/kg) every two days. The 4 bars in each group from left to right correspond to 1) Gpr132-WT/Veh, 2) Gpr132-WT/Rose, 3) Gpr132-KO/Veh, and 4) Gpr132-KO/Rose. (FIG. 5N) The ability of macrophage PPARγ deletion to exacerbate tumor growth in vivo was abolished in Gpr132-KO mice (n=4). DKO, mf-g/Gpr132 double KO. (O-P) Pharmacological Gpr132 inhibition impeded mammary tumor growth. Female mice (6-week-old) were treated with si-Gpr132 (n=8) or si-Ctrl (n=6) for 18 days via intravenous injection at 10 μg/mouse twice/week, 3 days before and 15 days after EO771 cell mammary fat pad injection. (FIG. 5O) Tumor volume was significantly decreased by si-Gpr132 treatment. (FIG. 5P) Gpr132 expression in tumors was effectively depleted. Error bars, SD; *, p<0.05; **, p<0.01; ***, p<0.005; ****, p<0.001; n. s. non-significant. (FIG. 5Q) A simplified model for how macrophage PPARγ inhibits inflammation and tumor growth by repressing the transcription of macrophage Gpr132, a novel pro-inflammatory and pro-tumor membrane receptor. Pharmacological PPARγ activation or Gpr132 blockade attenuates breast cancer progression. Moreover, both macrophage PPARγ and Gpr132 are key mediators of the anti-tumor effects of the clinically used TZD drug rosiglitazone.

FIGS. 6A-6D. Immunofluorescence staining of tumor sections. FIGS. 5A-6B, Macrophage recruitment was increased in both Tie2Cre-PPARγ-KO and LyzCre-PPARγ-KO mice compared to WT control mice. Tumor sections were stained for a macrophage marker F4/80 (n=4). FIGS. 6C-6D, Angiogenesis was increased in Tie2Cre-PPARγ-KO mice but not in LyzCre-PPARγ-KO mice. Tumor sections were stained for an endothelial marker endomucin (n=4). FIGS. 6A, 6C Representative images of staining; scale bars, 25 μm. b,d Quantification of the number of blood vessels. Error bars, SD.

FIG. 7. Expression of other candidates genes was unaltered in PPARγ-deficient macrophages. Candidate genes that encode membrane proteins and are potentially regulated by macrophage PPARγ were selected from published microarray databases. Their expression in bone marrow macrophages (BMMf) or spleen macrophages (SpMf) derived from mf-g-KO mice (second bar of each group) or littermate control mice (first bar of each group) were quantified by RT-QPCR (n=3). LGR5 and CCR4 expression was also examined, but the expression was too low to detect. Error bars, SD.

FIG. 8. Gpr132-null macrophages were refractory to the anti-tumor effects of rosiglitazone pre-treatment. Rosiglitazone pre-treatment of WT macrophages, but not Gpr132-KO macrophages, inhibited the growth of luciferase labeled 4T1.2 mouse mammary tumor cells in the co-cultures (n=3). Error bars, SD.

FIGS. 9A-9D. Tumor-derived factors activate M2-like macrophages via Gpr132. (FIG. 9A) The pH values of conditioned media (CM) from cancer cells or macrophages (n=5-8); *p<0.05, ****p<0.001 compared with RAW 264.7 macrophage CM. (FIG. 9B) Western blot for CD206 and Gpr132 in RAW 264.7 macrophages after treatment with indicated cancer cell CM for 24 hrs. Actin was used as a loading control. The CD206/actin or Gpr132/actin ratio was quantified and shown as fold changes compared to control (n=3); *p<0.05, ***p<0.005 compared with control. (FIG. 9C) Flow cytometry analysis of Gpr132 and CD206 in BMDMs with or without EO771 CM treatment. The experiments were repeated twice and the representative results are shown. (FIG. 9D) Immunofluorescence staining for CD11b in WT and Gpr132-KO BMDMs after differentiation in the absence or presence of 30% EO771 CM or 4T1.2 CM for 7 days. Elongated macrophage morphology indicates a M2-like phenotype. Nuclei were stained with DAPI. Scale bar, 25 μm. WT, wild-type; Gpr132-KO, Gpr132 knockout.

FIGS. 10A-10F. Lactate is a Gpr132 ligand/activator that induces macrophage M2 phenotype. (FIG. 10A) Lactate in the EO771<3 kDa CM bound to macrophage Gpr132. LC-MS was used to quantify lactate in the eluent of Gpr132 co-IP. (Left) Representative LC-MS chromatograms; (Right) Quantification of relative lactate binding (n=3). ****p<0.001 compared with WT. (FIG. 10B) Macrophage morphology after cancer cell CM treatment. 4T1.2 cells were cultured in the presence or absence of oxamic acid (90 mM) for 3 days. CM was harvested after the cells were cultured for another 24 hrs without oxamic acid. As a rescue, CM of oxamic acid-treated 4T1.2 cells was supplemented with exogenous lactate. Representative images of WT and Gpr132-KO BMDMs treated with indicated CM for 24 hrs are shown. Scale bar, 500 μm. (FIG. 10C) Western blot for CD206 in WT and Gpr132-KO BMDMs after treatment with lactate (5 mM) for 24 hrs. Actin was used as a loading control. CD206/actin ratio was quantified and shown as fold changes compared to control (n=3-4); *p<0.05 compared with WT control. (FIGS. 10D-10F) RT-qPCR analysis of the expression of PPARγ (FIG. 10D), GM-CSF (FIG. 10E) and CCL17 (FIG. 10F) in WT or Gpr132-KO BMDMs in the presence or absence of lactate (25 mM) for 6 hrs (n=3-4).

FIGS. 11A-11C. Lactate-activated macrophage promotes cancer cell adhesion, migration and invasion via Gpr132. (FIG. 11A) Adherence assays. 4T1.2 cells were suspended in fibronectin (10 mg/ml) pre-coated plates with CM from spleen-derived macrophages (mf) that were treated with 4T1.2 CM or lactate (5 mM) with or without Gpr132 antibody (6 mg/ml) or normal IgG (6 mg/ml) for 10 min. The adhered cells were stained with crystal violet, dissolved in 1% Triton X-100 and measured by OD590. (FIGS. 11B-11C) Boyden chamber assay of cancer cell migration and invasion. 4T1.2 cells (B) or EO771 cells (FIG. 11C) were plated on the upper chamber inserts, with untreated (Control), 4T1.2 CM-activated or lactate-activated spleen-derived WT or Gpr132-KO macrophages plated in the lower chambers. For invasion assay, the inserts were pre-coated with 60 μl matrigel. After migration for 6 hrs (FIG. 11B) or invasion for 24 hrs (FIG. 11C), the migrated or invaded cells were stained with crystal violet and counted as cells per field of view under microscope (n=3-4); For (FIG. 11C), the first bar of each data set of the bar graphs represents the control mf, and the second bar represents Gpr132KO; *p<0.05; **p<0.01; ***p<0.005; ****p<0.001. Scale bar, 500 μm.

FIGS. 12A-12L. Gpr132 deletion attenuates breast cancer lung metastasis by reducing M2 macrophages. (FIGS. 12A-12G) Lung metastases from breast cancer cells were decreased in Gpr132-KO mice. EO771 cells (FIGS. 12A-12C) or EO771-LMB cells (FIGS. 12D-12G) were transplanted into the mammary fat pad of WT and Gpr132-KO mice. Primary tumors were resected when reached 1500 mm3 (FIGS. 12A-12C) or 650 mm3 (FIGS. 12D-12G). After 25 days (FIGS. 12A-12C) or 16 days (FIGS. 12D-12G), lungs were harvested and subjected to H&E staining (FIGS. 12A, 12D). The number (FIGS. 12B, 12E) and size (FIGS. 12C, 12F) of tumor nodules were quantified from the stained lung sections (n=7-9). (FIG. 12G) Lung metastases were quantified using a fluorescent probe that selectively activates in tumors but not normal tissues by responding to low pH. In the EO771-LMB model, mice were intravenously injected with Probe 5c 24 hrs before lung dissection and image acquisition (n=2-3). (FIGS. 12H-12L) M2 macrophages in lung metastasis were reduced in Gpr132-KO mice. Immunohistochemistry for CD206 (FIG. 12H), as well as RT-qPCR for Arg-1 (FIG. 12I), CCL17 (FIG. 12J), CCL22 (FIG. 12K) and Ym-1 (FIG. 12L) in the lungs of WT or Gpr132-KO mice of EO771-LMB model (n=4); *p<0.05; ***p<0.005. Scale bar, 500 μm.

FIGS. 13A-13B. Gpr132 expression positively correlates with metastasis and M2 macrophage in human breast cancer. (FIG. 13A) Higher Gpr132 expression correlated with lower metastasis-free and relapse-free survival in breast cancer patients. Data were obtained from PrognoScan. (FIG. 13B) Linear regression analyses of TCGA-BRCA RNA-seq data showed that Gpr132 expression positively correlated with the expression of M2 macrophage markers, including CD163, CCL17, CCL22, CCR2, TLR1, TLR8, TGM2 and CD200R1, in human breast cancer lesions (n=1100).

FIG. 14. A working model for how lactate-Gpr132 axis facilitates a positive feedback loop between tumor and macrophage to promote breast cancer metastasis. Cancer cell-secreted lactate activates macrophage Gpr132 receptor to induce M2 polarization via enhancing AKT/mTOR signaling. In turn, lactate-activated macrophages facilitate breast cancer metastasis. Thus, Gpr132 functions as a macrophage sensor to relay the acidic signal in the tumor microenvironment to TAM activation and metastasis induction.

FIGS. 15A-15B. Cancer cell-derived factors promote macrophage M2 activation via Gpr132. (FIG. 15A) Western blot for CD206 and Gpr132 in RAW 264.7 macrophages after treatment with EO771 CM for 0-24 hrs. Actin was used as a loading control. CD206/actin or Gpr132/actin ratio was quantified and shown as fold changes compared to control (0 hr). *p<0.05, **p<0.01 compared with control (0 hr). (FIG. 15B) Western blot for arginase 1 (Arg-1) in WT and Gpr132-KO bone marrow-derived macrophages after treatment with 4T1.2 CM for 24 hrs. Actin was used as a loading control. WT, wild-type; Gpr132-KO, Gpr132 knockout.

FIGS. 16A-16C. The <3 kDa fraction of cancer cell CM displays lower pH and induces macrophage M2 phenotype. (FIG. 16A) The pH values in the whole media, <3 kDa fraction and >3 kDa fraction of RPMI-1640 control media or EO771 CM (n=3-4); ****p<0.001. (FIG. 16B) Western blot for CD206 in bone marrow-derived macrophages (BMDMs) after treatment with whole media, <3 kDa fraction or >3 kDa fraction of RPMI-1640 control media for 24 hrs. Actin was used as a loading control. CD206/actin ratio was quantified and shown as fold changes compared to the group treated with whole media. (FIG. 16C) Western blot for CD206 in WT and Gpr132-KO BMDMs after treatment with mock control, whole CM, <3 kDa fraction or >3 kDa fraction of CM for 24 hrs. Actin was used as a loading control. CD206/actin ratio was quantified and shown as fold changes compared to WT BMDM treated with mock control. CM, conditional media; WT, wild-type; Gpr132-KO, Gpr132 knockout.

FIGS. 17A-17E. Cancer cell-derived lipids reduce macrophage M2 phenotype independent of Gpr132. RT-qPCR analysis of the expression of Arg-1 (FIG. 17A), CCL17 (FIG. 17B), CCL22 (FIG. 17C), YM-1 (FIG. 17D) and YM-2 (FIG. 17E) in WT or Gpr132-KO spleen macrophages treated with cancer cell-derived lipids or vehicle control for 24 hrs (n=4). WT, wild-type; Gpr132-KO, Gpr132 knockout.

FIGS. 18A-18E. Lactate secreted by EO771 and 4T1.2 cells is responsible for the low pH in their CM. (FIG. 18A) Lactate levels in the conditional media (CM) from various cancer cells (n=3); *p<0.05, ****p<0.001 compared with EO771 CM; #### p<0.001 compared with 4T1.2 CM. (FIG. 18B) Lactate levels in EO771 CM at the indicated time point of culture (n=3). (FIG. 18C) Lactate levels in the whole CM, <3 kDa and >3 kDa fractions of CM from 24 h EO771 cell cultures (n=3). (FIGS. 18D-18E) EO771 and 4T1.2 cells were treated with or without oxamic acid (90 mM) for 3 days. CM was harvested after the cells were cultured for another 24 hrs without oxamic acid before lactate (FIG. 18D) and pH (FIG. 18E) quantification (n=3). **p<0.01, ***p<0.005, ****p<0.001.

FIGS. 19A-19B. Lactate activates macrophage M2 phenotype via Gpr132. (FIG. 19A) Macrophage morphology after cancer cell CM treatment. EO771 cells were cultured in the presence or absence of oxamic acid (90 mM) for 3 days. CM was harvested after the cells were cultured for another 24 hrs without oxamic acid. Representative images are shown for WT and Gpr132-KO bone marrow-derived macrophages treated with indicated CM for 24 hrs. Scale bar=500 μm. WT, wild-type; Gpr132-KO, Gpr132 knockout. (FIG. 19B) Western blot for CD206 in WT and Gpr132-KO spleen macrophages after treatment with lactate (5 mM) for 24 hrs. Actin was used as a loading control.

FIG. 20. Lactate activates macrophage AKT/mTOR signaling via Gpr132. WT and Gpr132-KO bone marrow-derived macrophages were serum starved for 24 hrs. After treatment with lactate (5 mM) for 15 min, cell lysate was harvested for detecting the activation status of AKT signaling pathway using a PathScan Akt Signaling Antibody Array Kit. WT, wild-type; Gpr132-KO, Gpr132 knockout.

FIG. 21A-21F. Gpr132 is not required for IL-4 induction of M2 macrophage. RT-qPCR analysis of the expression of Arg-1 (FIG. 21A), CCL17 FIG. 21B), CCL22 (FIG. 21C), PPARγ (FIG. 21D), YM-1 (FIG. 21E) and YM-2 (FIG. 21F) in WT or Gpr132-KO spleen macrophages treated with IL-4 (10 ng/ml) or vehicle control for 16 hrs (n=3). WT, wild-type; Gpr132-KO, Gpr132 knockout.

FIGS. 22A-22F. Gpr132 is not required for LPS induction of M1 macrophage. RT-qPCR analysis of the expression of iNOS (FIG. 22A), COX-2 (FIG. 22B), MCP-1 (FIG. 22C), TNFα (FIG. 22D), IL-1β (FIG. 22E) and IL-6 (FIG. 22F) in WT or Gpr132-KO bone marrow-derived macrophages treated with LPS (50 ng/ml) or vehicle control for 16 hrs (n=3-4). WT, wild-type; Gpr132-KO, Gpr132 knockout.

FIGS. 23A-23B. Cancer cell-derived lactate activates macrophages to promote cancer cell migration and invasion via Gpr132. (FIGS. 23A-23B) Boyden chamber assay. EO771 or 4T1.2 cells were cultured in the presence or absence of oxamic acid (90 mM) for 3 days. CM was harvested after the cells were cultured for another 24 hrs without oxamic acid. As a rescue, CM of oxamic acid-treated EO771 or 4T1.2 cells was supplemented with exogenous lactate. These CM were used to treat WT or Gpr132-KO bone marrow-derived macrophages plated in the lower chambers before EO771 cells (FIG. 23A) or 4T1.2 cells (FIG. 23B) were plated on the upper cell culture inserts. For invasion assay, the inserts were pre-coated with 60 μl matrigel. After migration for 6 hrs (FIG. 23A) or invasion for 24 hrs (FIG. 23B), the migrated or invaded cells were stained with crystal violet and counted as cells per field of view under microscope (n=3-4); for (FIG. 23A), the bars in each data set of the bar graph represent, from left to right, control, EO771, Oxamic acid, and Oxamic acid+Lactate; for (FIG. 23B), the bars in each data set of the bar graph represent, from left to right, control, 4T1.2CM, Oxamic acid, and Oxamic acid+Lactate; *p<0.05; ****p<0.001. Scale bar=500 μm. WT, wild-type; Gpr132-KO, Gpr132 knockout.

FIG. 24. Primary breast tumor enhances Gpr132 expression in distant metastatic site. EO771 cells were transplanted into the mammary fat pad of WT mice. When the primary tumors reached about 1500 mm3, lungs were harvested. Gpr132 expression in the lung of control and tumor-bearing mice was quantified by RT-qPCR (n=3-4).

FIG. 25. RT-qPCR analysis of Gpr132 and CD206 mRNA in BMDMs with or without EO771 CM treatment (n=3). *P<0.05. FIG. 27. Quantification of macrophage morphology (FIG. 9D) as an elongation factor (n=2-4). ****P<0.001.

FIG. 26. Quantification of macrophage morphology as an elongation factor (n=2-4). *P<0.05, ***P<0.001.

FIG. 27. Calcium mobilization triggered by lactate, but not HCl, was significantly impaired in Gpr132-KO BMDMs. WT or Gpr132-KO BMDMs were stimulated with 25 mM lactate (Left) or HCl (Right) (n=3).

FIG. 28. Quantification of macrophage morphology as an elongation factor (n=2-3). *P<0.05, ***P<0.005.

FIG. 29. RT-qPCR for Arg-1, GM-CSF, and CCL22, in the lungs of WT or Gpr132-KO mice of the EO771-LMB model (n=4). *P<0.05, ***P<0.005.

### DETAILED DESCRIPTION OF THE INVENTION

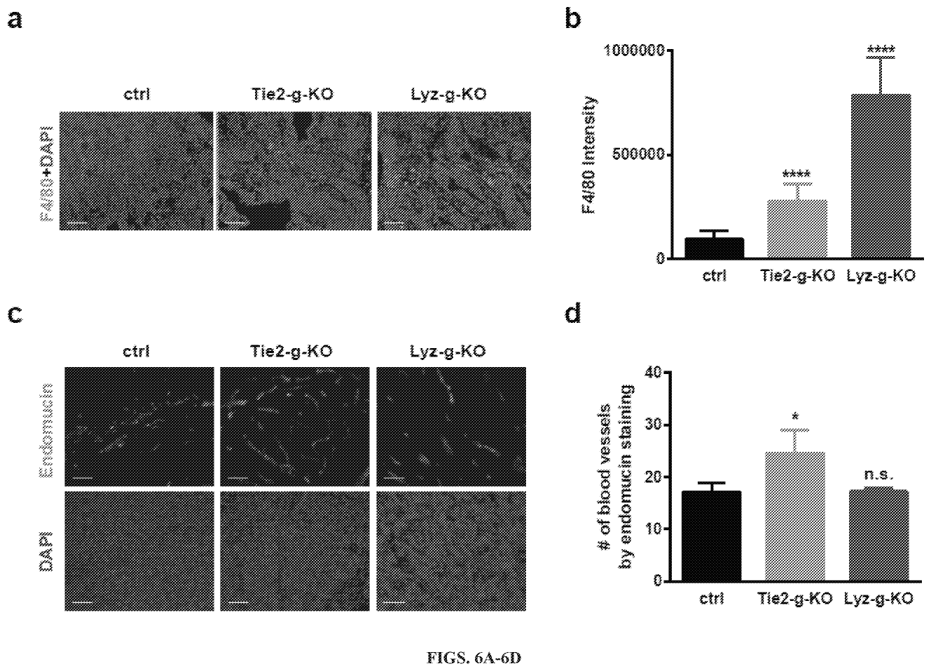

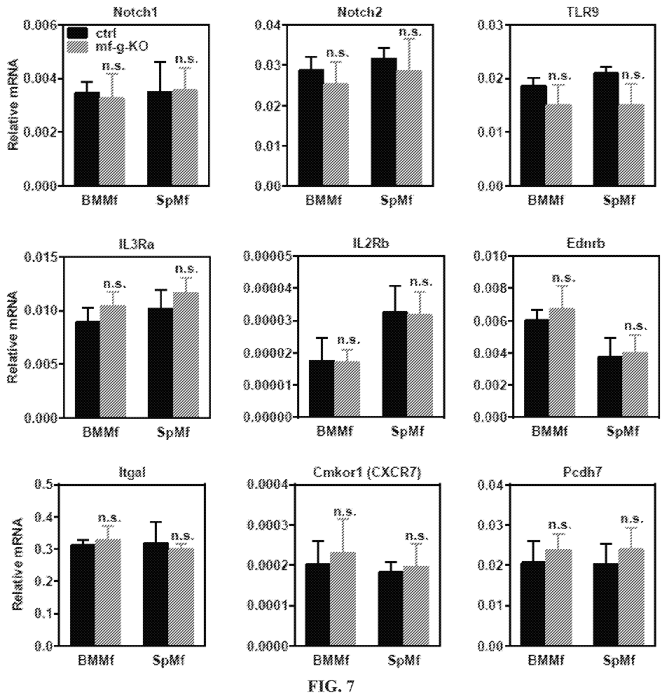

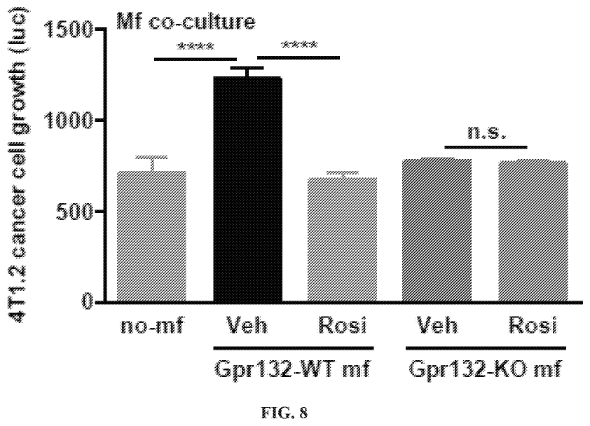

Given the pleotropic and important roles of PPARγ in physiology and disease, as well as the wide-spread usage of TZD drugs for the treatment of insulin resistance and type II diabetes, it is of paramount importance to elucidate the mechanisms for how PPARγ and TZDs affect cancer. Here, the inventors have determined a crucial yet previously unrecognized role of macrophage PPARγ in suppressing cancer progression and mediating the anti-tumor effects of rosiglitazone (FIG. 5Q). Mechanistically, PPARγ activation in macrophages tunes down inflammatory programs by repressing the transcription of GPR132, which is a pro-inflammatory membrane receptor (FIG. 5Q). As described in the examples, tumor growth is inhibited when macrophage GPR132 level is low by either GPR132 deletion/inhibition or PPARγ activation via rosiglitazone, whereas tumor growth is exacerbated when macrophage GPR132 level is high as the result of macrophage PPARγ deficiency. Importantly, GPR132 deletion abolishes the cancer regulation by macrophage PPARγ or rosiglitazone, indicating that GPR132 is an essential mediator of PPARγ functions in macrophages and tumor progression. These findings reveal PPARγ and GPR132 as fundamental key players in TAM, providing new mechanisms how macrophages interact with tumor cells to promote cancer malignancy.

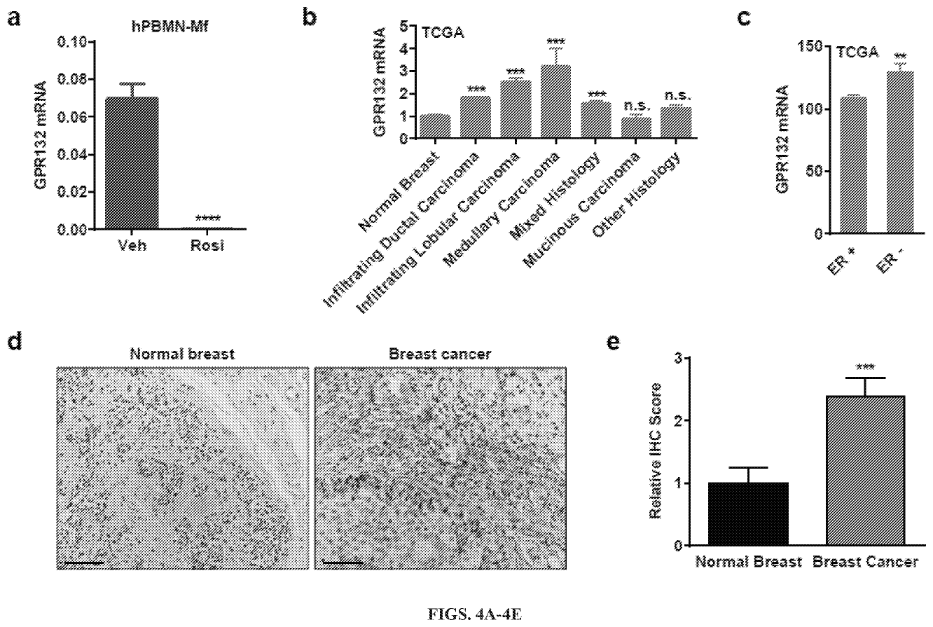

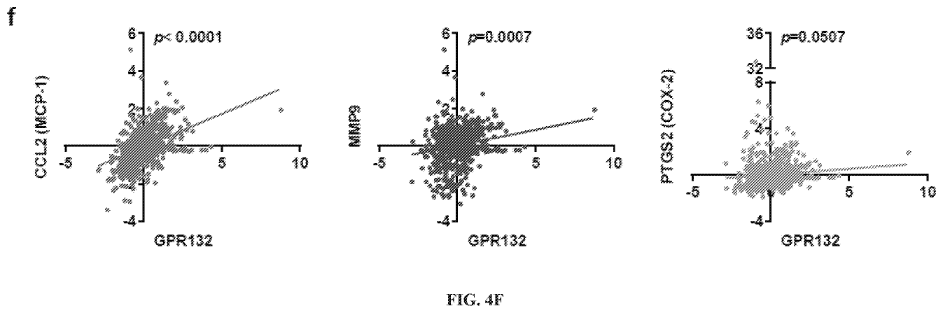

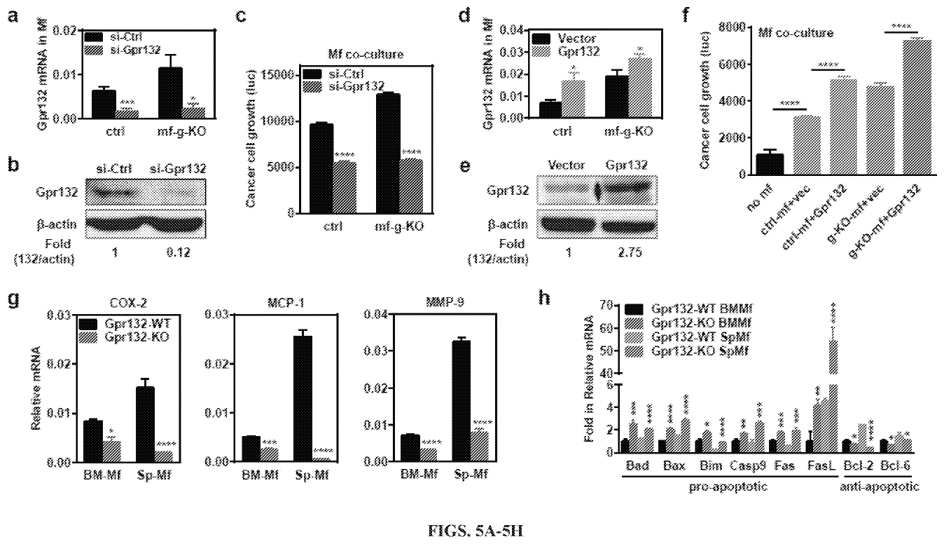

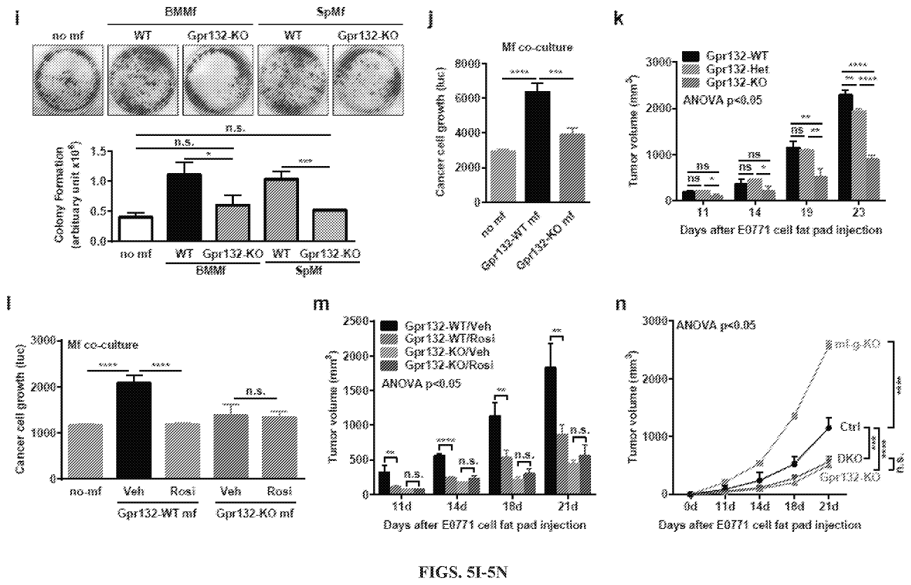

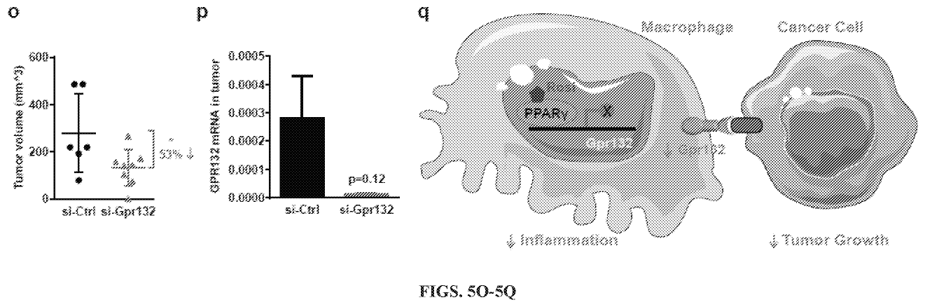

Cancer cells form an intimate relationship with TAMs to proliferate and survive. Targeting the infiltrating macrophages to alter their number and properties can lead to a significant inhibition of cancer malignancy. The data of the examples shows that this can be achieved by GPR132 inhibition in the macrophage. By elucidating the mechanisms that macrophages use to promote cancer and inflammation, effective diagnostic tools as well as innovative anti-tumor and anti-inflammatory therapeutics can be designed. For example, macrophage levels of PPARγ and GPR132 may predict not only tumor aggressiveness but also the pharmacological responses to rosiglitazone or GPR132 inhibitors. The inventors' findings may explain why rosiglitazone exerts anti-tumor effects in certain cancers but not others—cancers with abundant PPARγ-positive macrophages may be sensitive, whereas cancers with limited macrophages or PPARγ-negative macrophages may be resistant. Moreover, the positive association of GPR132 with inflammation and breast cancer in human (FIG. 4B-F), the repression of GPR132 expression by rosiglitazone in human macrophage (FIG. 4A) and the anti-tumor effects of pharmacological GPR132 inhibition (FIG. 5O-P) demonstrate the usefulness of GPR132 blockade as a new therapeutic.

Furthermore, recent studies have shown that cancer cell-derived lactate can educate macrophages to functional TAMs, which in turn promotes tumor growth. How lactate activation of TAMs affects cancer metastasis is poorly understood. Importantly, the molecular basis by which macrophages sense and respond to lactate is largely unknown. The inventors found that cancer cell-derived lactate is a novel Gpr132 ligand/activator that facilitates macrophage M2 phenotype by enhancing the AKT/mTOR signaling pathway in a Gpr132-dependent manner. As a result, Gpr132 deletion impairs macrophage M2 activation and breast cancer metastasis in vitro and in vivo. These findings not only decipher the roles and mechanisms of Gpr132 in macrophage and cancer metastasis, but also provide evidence for Gpr132 as a macrophage sensor/receptor for lactate. Collectively, the studies described in the examples of the application reveal novel molecular basis for the vicious cycle between cancer cells and macrophages, and uncover Gpr132 as an exciting new therapeutic target for cancer metastasis.

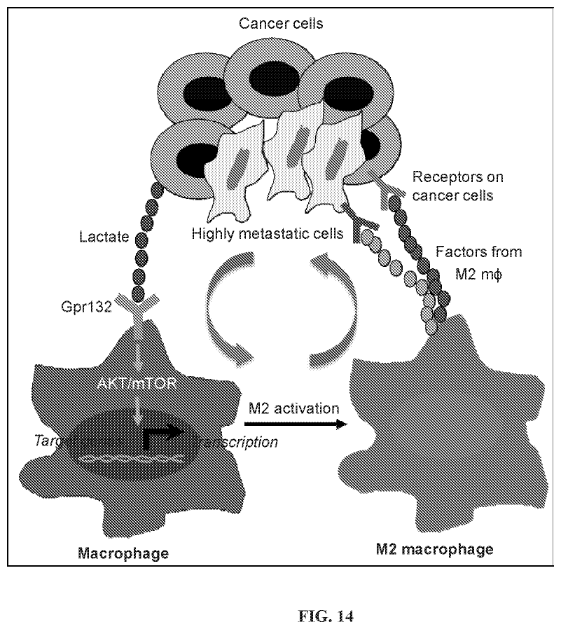

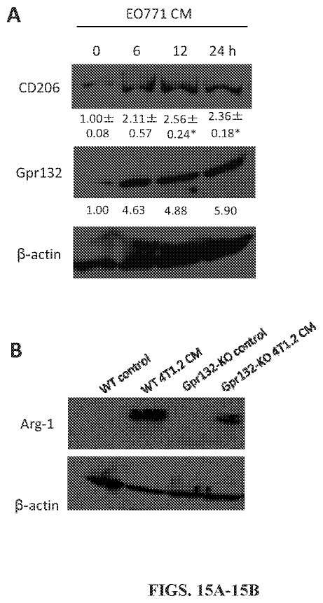

TAMs are generally biased toward M2 phenotype and play a critical role in cancer metastasis. However, precisely how TAMs are educated by breast cancer cells is still poorly defined. In the present study, lactate-Gpr132 was identified as a key signal and receiver pair that represents a critical mechanism for TAM polarization and breast cancer metastasis. The examples show that cancer cell-derived lactate activates macrophage Gpr132 to promote M2 phenotype. In turn, lactate-activated macrophages enhance cancer cell adhesion, migration and invasion in vitro and metastasis in vivo, forming a positive feedback loop (FIG. 14). Importantly, the examples provide evidence that Gpr132 is a novel lactate receptor/sensor in macrophage that is essential for TAM education by cancer cells. Disruption of this lactate-Gpr132 axis abrogates TAM polarization and breast cancer lung metastasis in mice, and lower Gpr132 expression correlates with better survival in breast cancer patients. Therefore, the data of the examples reveal novel tumor-macrophage interplay during cancer metastasis, and provide new biological insights to tumor immunity and breast cancer intervention.

During tumor progression, the recruited macrophages are usually polarized towards M2 phenotypes by responding to cancer cell-secreted factors such as M-CSF and GM-CSF. Therefore, the specific receptors on macrophages are crucial for sensing these stimuli and TAM polarization. Indeed, it has been shown that inhibition of CSF-1R on macrophages impairs M2 polarization and cancer progression. The examples show in vitro that cancer cell CM not only increases the expression of Gpr132 on macrophages but also promotes macrophage M2 phenotype in a Gpr132-dependent manner; and also show in vivo that primary mammary tumor not only augments the expression of Gpr132 in distant metastatic sites but also develop spontaneous lung metastasis in a Gpr132-dependent manner. As a result, loss of Gpr132 in the tumor environment abrogates both macrophage M2 activation and breast cancer lung metastasis in mice; reduced Gpr132 expression correlates with less M2 TAMs and better prognosis with increased metastasis- and relapse-free survival in human breast cancer patients. These new findings reveal Gpr132 as a previously unrecognized key macrophage receptor for cancer cell signals that contribute to cancer cell education of TAMs. The inventors' work reinforces the concept that macrophages are entrained by cancer cells, and expands the molecular understanding of the signals and receivers mediating TAM polarization.

Gpr132 has been implicated as a member of pH-sensing G protein-coupled receptor family. In the experiments described in the examples, the pH value in the CM of a panel of cancer cell lines was screened, and their effects on M2 macrophage activation was examined. The examples show that CM of EO771 and 4T1.2 cells, which exhibit lower pH and higher levels of lactate than CM from other cells, stimulate macrophage M2 phenotype via Gpr132. It was found that this Gpr132-dependent activity resides in the <3 kDa fraction of the CM and is largely attributed to lactate rather than lipids. Moreover, the LC-MS analysis confirmed Gpr132 binding to CM lactate. These findings support lactate as a key cancer cell-derived ligand/activator for Gpr132 that triggers TAM polarization, whereas other reported Gpr132 ligands such as 9-Hydroxyoctadecadienoic acid may be less important in this context. Moreover, Gpr81 has also been reported to be a lactate receptor that inhibits adipose lipolysis and promote cancer cell survival. However, Gpr81 is specifically expressed in mesenchymal and epithelial lineages such as adipocytes and cancer cells but absent in macrophages. In contrast, Gpr132 is exclusively expressed in macrophage and other hematopoietic lineages but absent in adipocytes or cancer cells. This suggests that lactate engages different GPCRs in distinct cell types to perform diverse functions. Therefore, the findings described in the examples identify Gpr132 as a macrophage lactate receptor. This work opens an exciting new path to future investigations on the functional roles of the lactate-Gpr132 axis in the crosstalk between metabolism and immunity.

The examples demonstrate that the pH reduction in EO771 CM was prevented after blocking lactate production using oxamic acid, confirming that rising lactate was the main cause of the acidic cancer environment (FIG. 18E). The data described in the examples not only confirms previous findings that lactate is a key cancer signal that entrains TAMs, but also identifies Gpr132 as a key lactate sensor/receiver on macrophages.

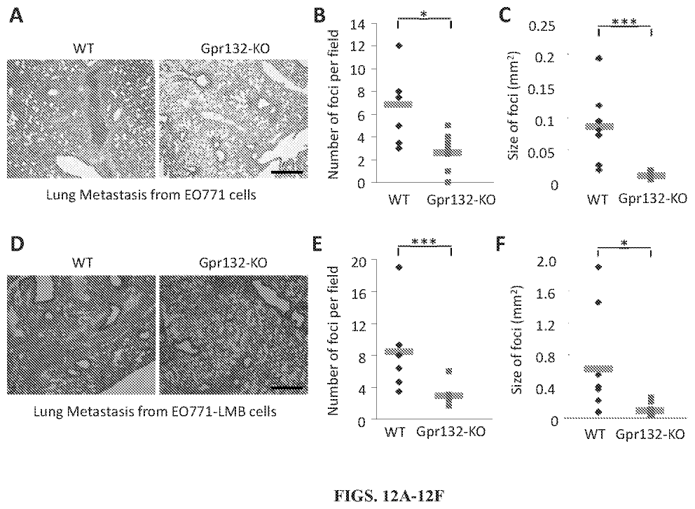

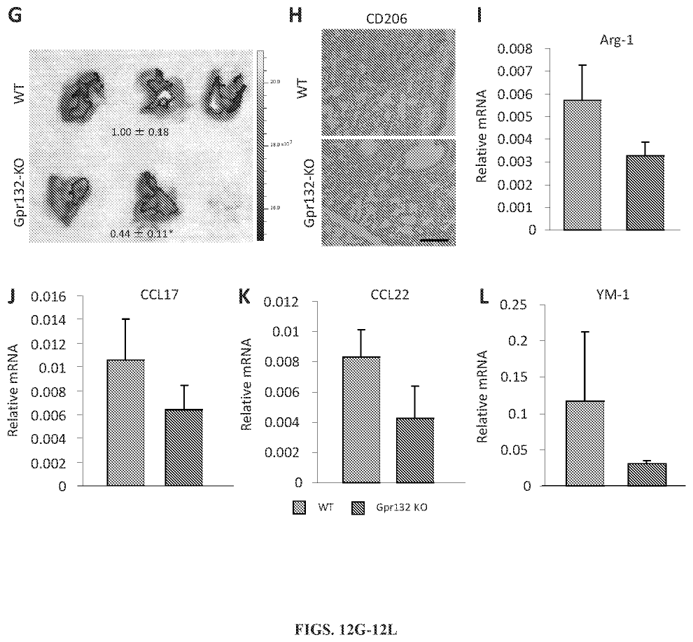

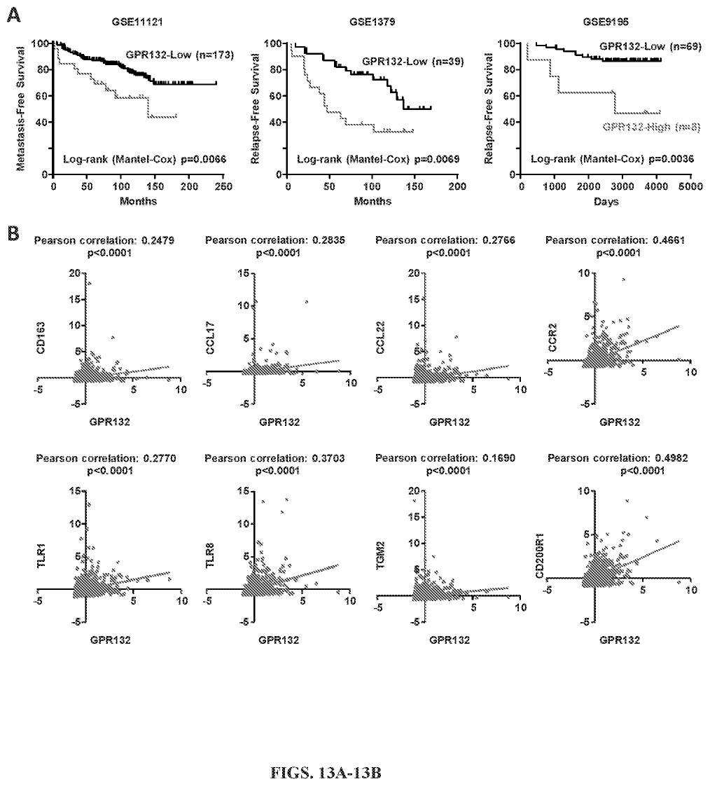

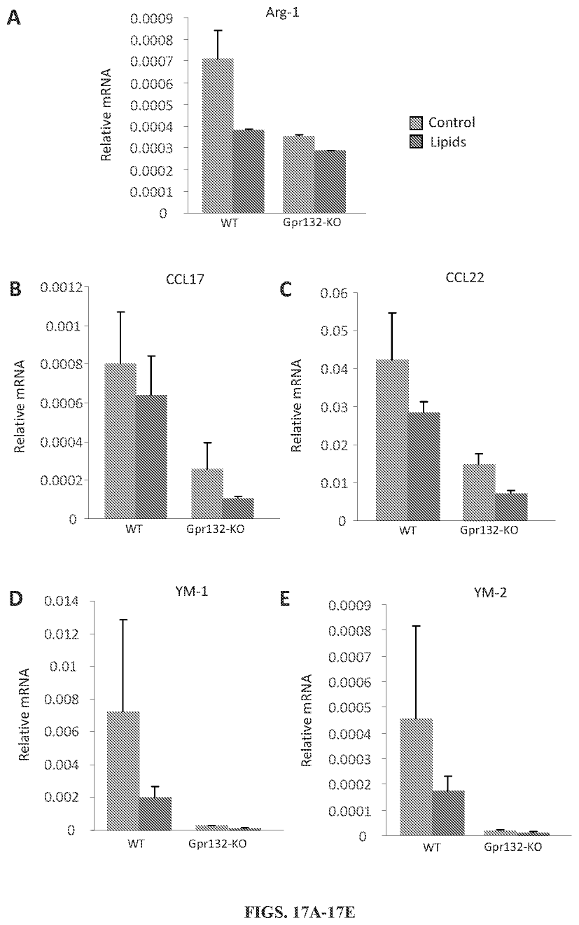

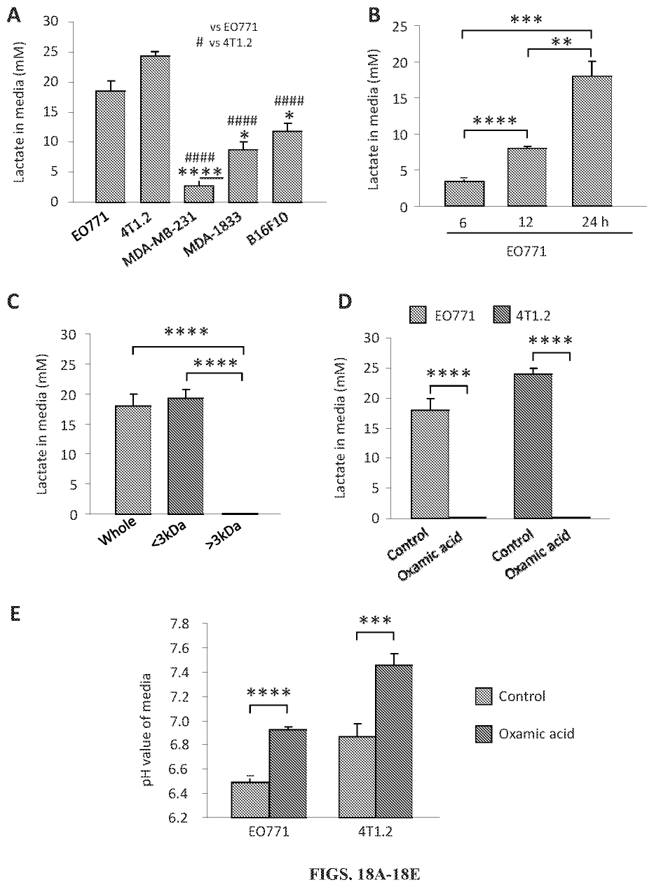

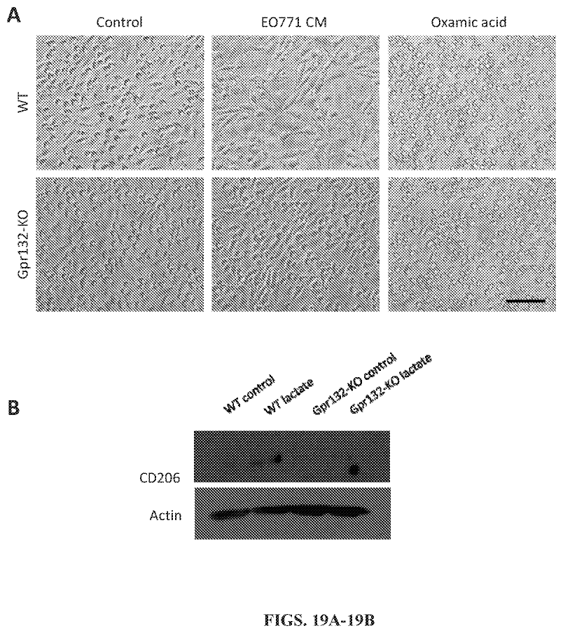

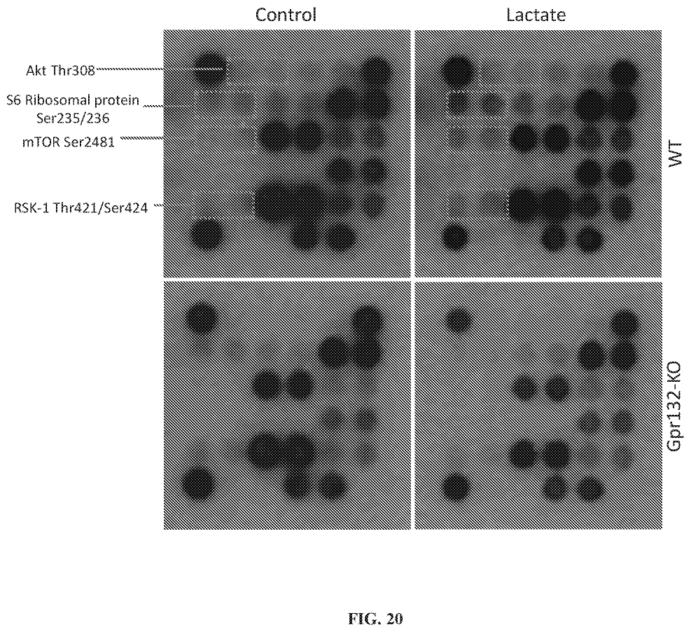

Macrophage M2 activation is mainly controlled by a small group of signaling pathways. The screen using AKT antibody array revealed that lactate enhances macrophage AKT/mTOR signaling in a Gpr132-dependent manner, providing important mechanisms for lactate-Gpr132 functions. In addition, lactate education of M2 macrophages may involve the induction of Arg-1 and the hypoxia-inducible factor 1α (HIF1α)-vascular endothelial cell growth factor (VEGF) pathway (14). As the examples show that Gpr132 is a receptor/sensor of lactate, it is plausible that HIF1α and Arg-1 induction are also part of the downstream events of Gpr132. Indeed, Gpr132-KO macrophages (FIG. 17A) and lung metastasis in Gpr132-KO mice (FIG. 12I) showed lower Arg-1 expression. Future studies are required to further delineate the detailed downstream signals triggered by lactate activation of Gpr132.

The co-migrating tumor cells and macrophages depend on each other for cancer metastasis. The examples show that lactate from cancer cells and Gpr132 on macrophages form a ligand-receptor/signal-receiver pair to activate M2 macrophages, which in turn stimulates cancer cell migration and invasion in a paracrine fashion, thereby inducing a positive feedback loop to promote breast cancer metastasis (FIG. 14). As reported in several previous studies, activation of M2 macrophages may stimulate cancer metastasis via multiple cytokines such as CCL17, CCL18, CCL22, IL-10, VEGF, and transforming growth factor β (TGFβ). Therefore, the inventors' study further extends the knowledge and highlights the importance of this vicious cycle in cancer metastasis. Indeed, the inventors' in vivo findings demonstrate that blockade of this vicious circle by Gpr132 deletion impairs breast cancer lung metastasis by reducing M2 macrophages; their analysis of breast cancer patient data reveal that Gpr132 expression is positively correlated with M2 macrophages and poor prognosis. These findings provide support for the use of Gpr132 inhibitors as a cancer therapeutic and of Gpr132 as a cancer prognostic marker.

### I. GPR132

G protein coupled receptor 132 (GPR132), also termed G2A, is classified as a member of the proton sensing G protein coupled receptor (GPR) subfamily. Like other members of this subfamily, i.e., GPR4, OGR1 (GPR68), and TDAG8 (GPR65), G2A is a G protein coupled receptor that resides in the cell surface membrane, senses changes in extracellular pH and can alter cellular function as a consequence of these changes.

The human protein sequence of GPR132 is exemplified by the following: mcpmllkngy ngnatpvttt apwaslglsa ktcnnvsfee srivlvvvys avctlgvpan cltawlallq vlqgnvlavy llclalcell ytgtlplwvi yirnqhrwtl gllackvtay iffcniyvsi lflcciscdr fvavvyales rgrrrrrtai lisacifilv givhypvfqt edketcfdml qmdsriagyy yarftvgfai plsiiaftnh rifrsikqsm glsaaqkakv khsaiavvvi flvcfapyhl vllvkaaafs yyrgdrnamc gleerlytas vvflclstvn gvadpiiyvl atdhsrqevs rihkgwkews mktdvtrlth srdteelqsp valadhytfs rpvhppgspc pakrlieesc.

The human mRNA sequence is exemplified by the following (SEQ ID NO:1):

atgctgtcat caccagtaag ataccccagc ccggttggct aacccctagc tcagccctgt ttgtggttag gggagttcta
gacagaacat aattagaccc ctgctacttc ctgaaacctc agctaggact gcagggaggg gtgcgaggct agccacgcag
gcggggccct ggctttataa aggtataatt gacaaacaag aagagtacct aggccaggtg cggtggctta cgcctgtaat
cccagcactt tgggaggccg aggtcatttt aaactctcag agtgaacgtc ttgataggac cgacaagacg catgacatgt
acttagaaag cttatcttag agccacactg agattggaac ccgcaaaata tgccagggag gaaggtgagc aagggacacg
acactcaccc ggagaaaccc agcaagcgca gcgaggctgt ggggagaccg gagccctgca caccgccggg
ggaaggtggg ccagcgccac caccgtggag aacagcgcgg aggcacccca cgagatgaga cggaactgcc
gtgagatcca gcaatgccaa ctgtgggtct gacccaggag aacggaaagc agggacgtga acagccctcc tcatgttctt
gacaccgtca ttctcagcag ctcagctaag gcacagaggc agccgagcgt ctgtcagcgg agtcgtggct gagcagaaca
cgccacacgc cacacgccac acgccacacg tgcaggattg ctcaagatgg aagggcacag tggaatatat atatatattt
atatttttgg cgagaccctg gaggacacac tgaatacaat ggaataccat cccgcctttg aaaggaaggg aaatcctggc
acacgctgca acaggaggga gcttgaggac actgtggtga gtggagcacg tgagacacgg aaggacacac gctgaagaca
cgcagagatg cccacccacg tggggaggtg acaggggagc ccagcgcaca gagacaaagt ggaatggagg
cctgggggct gggagcaaat gcggagcgag tgcttcctgg ggcagagtct ccgtttggga agatgagaag gttctgccga
cggatgctgg cgatggttgc agaagaatgt gaatgtgccc aatgctactg aaaaacggtt acaatggaaa cgccacccca
gtgaccacca ctgccccgtg ggcctccctg ggcctctccg ccaagacctg caacaacgtg tccttcgaag agagcaggat
agtcctggtc gtggtgtaca gcgcggtgtg cacgctgggg gtgccggcca actgcctgac tgcgtggctg gcgctgctgc
aggtactgca gggcaacgtg ctggccgtct acctgctctg cctggcactc tgcgagctgc tgtacacagg cacgctgcca
ctctgggtca tctatatccg caaccagcac cgctggaccc taggcctgct ggcctgcaag gtgaccgcct acatcttctt
ctgcaacatc tacgtcagca tcctcttcct gtgctgcatc tcctgcgacc gcttcgtggc cgtggtgtac gcgctggaga
gtcggggccg ccgccgccgg aggaccgcca tcctcatctc cgcctgcatc ttcatcctcg tcgggatcgt tcactacccg
gtgttccaga cggaagacaa ggagacctgc tttgacatgc tgcagatgga cagcaggatt gccgggtact actacgccag
gttcaccgtt ggctttgcca tccctctctc catcatcgcc ttcaccaacc accggatttt caggagcatc aagcagagca
tgggcttaag cgctgcccag aaggccaagg tgaagcactc ggccatcgcg gtggttgtca tcttcctagt ctgcttcgcc
ccgtaccacc tggttctcct cgtcaaagcc gctgcctttt cctactacag aggagacagg aacgccatgt gcggcttgga
ggaaaggctg tacacagcct ctgtggtgtt tctgtgcctg tccacggtga acggcgtggc tgaccccatt atctacgtgc
tggccacgga ccattcccgc caagaagtgt ccagaatcca taaggggtgg aaagagtggt ccatgaagac agacgtcacc
aggctcaccc acagcaggga caccgaggag ctgcagtcgc ccgtggccct tgcagaccac tacaccttct ccaggcccgt
gcacccacca gggtcaccat gccctgcaaa gaggctgatt gaggagtcct gctgagccca ctgtgtggca gggggatggc
aggttggggg tcctggggcc agcaatgtgg ttcctgtgca ctgagcccac cagccacagt gcccatgtcc cctctggaag
acaaactacc aatttctcgt tcctgaagcc actccctccg tgaccactgg ccccaggctt tcccacatgg aaggtggctg
catgccaagg ggaggagcga cacctccagg cttccgggag cccagagagc atgtggcagg cagtggggcc tcttcatcag
cagcctgcct ggctggctcc cttggctgtg ggcaggtagc acgcctgctg gcagaggtac ctggtggctg ccctgttcgc
atcagtggcg atgactttat ttgcggagca tttctgcaag cgttgcctgg atgcggtggt gcattgtggg ccctctgggc
tcctgcctca gaatgtcagt gagcaccatg ctggaggtca cccagcactg tggcagcgcc caggagggca tagggcagcc
taccacctcc aagggggcag gcgccctcat ctggggttgg gtctgtgctg agctggaggg cctctaggga accgtggggc
agggtggcca gctgctggct cccagagcgc agcccaggcg tcctcaacgg ggagccccaa atgtccacgc ccagaacaac
agttggcagg acaggtgtga cacagccaca gcagaggcaa ggggtgccag gagtccccag cggcatcctc ggggagatgc
tggtgagggg tccgtacagg gtggggtccc cacccctagc cccttactga ggggggagtg cagcagttgg cctgcttgtc
tggcggagaa agccagctcc ctgcaccctc ggggctgagt cagatctggg tctgccgcaa aggccttgcc tagaccaggt
cacactgatg ccctggtttc cctatctgta aaatggggcc aatgacacct acctcactgg gtcaccatcg agatcaatcc
tcctccctgc cccgacacct cgggcacatc gcatgcactc agagcacaga gccgggcaga cgcagcacct gcatggggag
cccagtgccc ggcacagcac aggggcttcc agggaggccg cgcagggccg tggggctgag ccacgctctc gttttgtcag
gcagctatgc agttgctctt ccttgttttt gttttgtttt tgtttttgtt tttaatattt atttttttag agacagggcc ttgctctgtt
gcctgggctg gagaacagtg gcaccatcat agctcactgc agcctcaaac tcctgggctc aagcgatcct ccccgctcag
cctcctgagt agctgggact acaggtgtgc accaccacac ccagccaaaa cagccatcct ccccttgaga gtcatcagaa
aaatacatta ggaaaatgtg tttagaaata aaagcacaag gcagggcagt gctcacgcct gtcatcccag cactttggga
ggccgagacg ggaggatcag ttgaggtcag gagtttgaga ccagcctcgg caacatggca aaatcttgtc tctttttttt
ggtattaaaa aaatcataaa aataaaagaa ataatgcaat ttaaccttca aaaaaaaa.

### II. GPR132 INHIBITORS

A GPR132 inhibitor may refer to any member of the class of compound or agents having an IC50 of 100 μM or lower concentration for a GPR132 activity, for example, at least or at most or about 200, 100, 80, 50, 40, 20, 10, 5, 1 μM, 100, 10, 1 nM or lower concentration (or any range or value derivable therefrom) or any compound or agent that inhibits the expression of GPR132. Examples of GPR132 activity or function may include, but not be limited to, the regulation and/or sensing of extracellular pH, and/or the binding of ligand. In some embodiments, the inhibition can be a decrease as compared with a control level or sample. In further embodiments, functional assay such as extracellular pH calculation, MTT assay, colony formation assay, invasion assay, apoptosis assay, or cell cycle analysis may be used to test the GPR132 inhibitors. In some embodiments, the GPR132 inhibitor is lysophosphatidylcholine, described in Murakami et al., The J. of Biological Chemistry, 2004, 279: 42484-42491, which is incorporated by reference.

GPR132 inhibitory agents include, without limitation, blocking antibodies, small molecular compounds (e.g., organic compounds, peptides, and peptide mimetics), antisense nucleic acids, ribozymes, and interfering RNAs. The antagonists may reduce GP132 activity levels by at least 50% (e.g., at least 60%, 70%, 80%, or 90%).

### III. GPR132 INHIBITORY NUCLEIC ACIDS

Inhibitory nucleic acids or any ways of inhibiting gene expression of GPR132 known in the art are contemplated in certain embodiments. Examples of an inhibitory nucleic acid include but are not limited to siRNA (small interfering RNA), short hairpin RNA (shRNA), double-stranded RNA, an antisense oligonucleotide, a ribozyme, and a nucleic acid encoding thereof. An inhibitory nucleic acid may inhibit the transcription of a gene or prevent the translation of a gene transcript in a cell. An inhibitory nucleic acid may be from 16 to 1000 nucleotides long, and in certain embodiments from 18 to 100 nucleotides long. The nucleic acid may have nucleotides of at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 or any range derivable therefrom.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory nucleic acids are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory nucleic acid may be capable of decreasing the expression of GPR132 by at least 10%, 20%, 30%, or 40%, more particularly by at least 50%, 60%, or 70%, and most particularly by at least 75%, 80%, 90%, 95% or more or any range or value in between the foregoing.

In further embodiments, there are synthetic nucleic acids that are GPR132 inhibitors. An inhibitor may be between 17 to 25 nucleotides in length and comprises a 5′ to 3′ sequence that is at least 90% complementary to the 5′ to 3′ sequence of a mature GPR132 mRNA. In certain embodiments, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature GPR132 mRNA, particularly a mature, naturally occurring mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA. Nucleic acid inhibitors such as siRNAs are commercially available. For example, ThermoFischer Scientific™ sells multiple different GPR132 siRNAs under catalog number AM16708. Santa Cruz Biotechnologies also provides commercially available GPR132 nucleic acids such as siRNAs, shRNA, and Lentiviral Particle Gene Silencers under catalog number sc-43776.

### IV. GPR132 INHIBITORY POLYPEPTIDES

In certain embodiments, an antibody or a fragment thereof that binds to at least a portion of GPR132 protein and inhibits GPR132 activity and/or function is used in the methods and compositions described herein.

In some embodiments, the anti-GPR132 antibody is a monoclonal antibody or a polyclonal antibody. In some embodiments, the antibody is a chimeric antibody, an affinity matured antibody, a humanized antibody, or a human antibody. In some embodiments, the antibody is an antibody fragment. In some embodiments, the antibody is a Fab, Fab′, Fab′-SH, F(ab′)2, or scFv. In one embodiment, the antibody is a chimeric antibody, for example, an antibody comprising antigen binding sequences from a non-human donor grafted to a heterologous non-human, human or humanized sequence (e.g., framework and/or constant domain sequences). In one embodiment, the non-human donor is a mouse. In one embodiment, an antigen binding sequence is synthetic, e.g., obtained by mutagenesis (e.g., phage display screening, etc.). In one embodiment, a chimeric antibody has murine V regions and human C region. In one embodiment, the murine light chain V region is fused to a human kappa light chain or a human IgG1 C region.

Examples of antibody fragments include, without limitation: (i) the Fab fragment, consisting of VL, VH, CL and CH1 domains; (ii) the “Fd” fragment consisting of the VH and CH1 domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VH domain and a VL domain are linked by a peptide linker which allows the two domains to associate to form a binding domain; (viii) bi-specific single chain Fv dimers (see U.S. Pat. No. 5,091,513) and (ix) diabodies, multivalent or multispecific fragments constructed by gene fusion (U.S. Patent Pub. 2005/0214860). Fv, scFv or diabody molecules may be stabilized by the incorporation of disulphide bridges linking the VH and VL domains. Minibodies comprising a scFv joined to a CH3 domain may also be made (Hu et al, 1996).

A monoclonal antibody is a single species of antibody wherein every antibody molecule recognizes the same epitope because all antibody producing cells are derived from a single B-lymphocyte cell line. Hybridoma technology involves the fusion of a single B lymphocyte from a mouse previously immunized with a GPR132 antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced. However, in therapeutic applications a goal of hybridoma technology is to reduce the immune reaction in humans that may result from administration of monoclonal antibodies generated by the non-human (e.g., mouse) hybridoma cell line.

Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDR is derived from mouse monoclonal antibodies, and the framework regions are derived from human amino acid sequences. It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

It is possible to create engineered antibodies, using monoclonal and other antibodies and recombinant DNA technology to produce other antibodies or chimeric molecules which retain the antigen or epitope specificity of the original antibody, i.e., the molecule has a binding domain. Such techniques may involve introducing DNA encoding the immunoglobulin variable region or the CDRs of an antibody to the genetic material for the framework regions, constant regions, or constant regions plus framework regions, of a different antibody. See, for instance, U.S. Pat. Nos. 5,091,513, and 6,881,557, which are incorporated herein by this reference.

By known means as described herein, polyclonal or monoclonal antibodies, binding fragments and binding domains and CDRs (including engineered forms of any of the foregoing), may be created that are specific to GPR132 protein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. Particularly, the antibodies may be ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by this reference. These techniques are further described in: Marks (1992); Stemmer (1994); Gram et al. (1992); Barbas et al. (1994); and Schier et al. (1996).

Methods for producing polyclonal antibodies in various animal species, as well as for producing monoclonal antibodies of various types, including humanized, chimeric, and fully human, are well known in the art. Methods for producing these antibodies are also well known. For example, the following U.S. patents and patent publications provide enabling descriptions of such methods and are herein incorporated by reference: U.S. Patent publication Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent publications, and other publications cited herein and therein are hereby incorporated by reference in the present application.

It is fully expected that antibodies to GPR132 will have the ability to neutralize or counteract the effects of the GPR132 regardless of the animal species, monoclonal cell line or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into binding fragments having the binding domain or CDR. Removal of the Fc portion reduces the likelihood that the antigen binding fragment will elicit an undesirable immunological response and, thus, antibodies without Fc may be particularly useful for prophylactic or therapeutic treatments. As described above, antibodies may also be constructed so as to be chimeric, partially or fully human, so as to reduce or eliminate the adverse immunological consequences resulting from administering to an animal an antibody that has been produced in, or has sequences from, other species.

Exemplary GPR132 inhibitory inhibitory polypeptides include, for example, anti-GPR132 antibodies such as antibodies sold under catalog numbers LS-C383594, LS-C177094, LS-C120683, LS-A1604, LS-A3701, LS-C353432, LS-C383761, LS-C292007, LS-C199008, LS-C120683, LS-A1600, LS-A3702, LS-A1605 available commercially from LifeSpan BioSciences, antibody Nos. ABIN615221, ABIN1086357, ABIN302435, ABIN636864, ABIN317643, ABIN213446, ABIN2929966, ABIN2892423, ABIN2270799, ABIN2704849, ABIN2765342, ABIN2604388, ABIN2270648, ABIN2437881, ABIN270951, ABIN2439526, ABIN2284554, ABIN2142750, ABIN2469367, and ABIN2890843 from antibodies online.com, NBP1-89808, NBP1-89807, NBP1-02347, NLS1600, and NLS3702 antibodies from NOVUS Biologicals, antibodies sold under catalog numbers orb223372, orb159220, orb84976, orb85083, orb161204, orb85489, orb85082, orb85490, and orb227391 available commercially from Biorbyt, HPA029694 and HPA029695 antibodies from Atlas Antibodies, antibodies sold under catalog numbers 48-227 and 51-248 from ProSci, antibodies sold under catalog numbers MBS854570 and MB S8505622 from MyBioSource.com, antibodies STJ93333 and STJ93172 available commercially from St. John's Laboratory, AP01208PU-N and AP06923PU-N antibodies from OriGene, antibody available under catalog number 119-10449 from Genway Biotech Inc., and antibody available under catalog number GWB-6E22C7 from Raybiotech, Inc.

A further example is a GPR132 blocking peptide available commercially from antibodies.com under product number ABIN3006625.

### V. GPR132 INHIBITORY SMALL MOLECULES

As used herein, a “small molecule” refers to an organic compound that is either synthesized via conventional organic chemistry methods (e.g., in a laboratory) or found in nature. Typically, a small molecule is characterized in that it contains several carbon-carbon bonds, and has a molecular weight of less than about 1500 grams/mole. In certain embodiments, small molecules are less than about 1000 grams/mole. In certain embodiments, small molecules are less than about 550 grams/mole. In certain embodiments, small molecules are between about 200 and about 550 grams/mole. In certain embodiments, small molecules exclude peptides (e.g., compounds comprising 2 or more amino acids joined by a peptidyl bond). In certain embodiments, small molecules exclude nucleic acids.

For example, a small molecule GPR132 inhibitory may be any small molecules that is determined to inhibit GPR132 function or activity. Such small molecules may be determined based on functional assays in vitro or in vivo.

Exemplary compounds include:

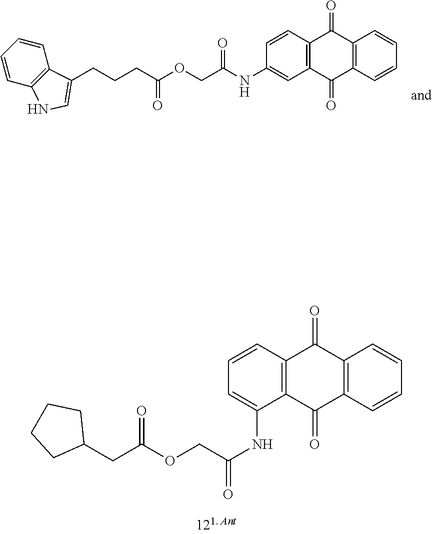 [SMILES: O=C(COC(=O)CCCc1c[nH]c2ccccc12)Nc1ccc2c(c1)C(=O)c1ccccc1C2=O ; O=C(COC(=O)CC1CCCC1)Nc1cccc2c1C(=O)c1ccccc1C2=O]

These compounds are further described in Shehata et al., RSC Adv., 2015, 5, 48551, which is herein incorporated by reference. Another further compound includes lysophosphatidylcholine (PMID: 15280385).

### VI. THERAPEUTIC METHODS

The methods described herein may be used to treat or prevent cancers by inhibition of GPR132 in the tumor microenvironment.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The cancers amenable for treatment include, but are not limited to, cancers and tumors of all types, locations, sizes, and characteristics. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

It is also contemplated that the compositions and methods described herein may be used to treat or ameliorate a number of immune-mediated or autoimmune disorders including, but not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes). Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis. In some embodiments, the compositions and methods described herein are used to treat an inflammatory component of a disorder listed herein.

### VII. PHARMACEUTICAL COMPOSITIONS

Embodiments include methods for treating cancer with compositions comprising a GPR132 inhibitor. Administration of the compositions will typically be via any common route. This includes, but is not limited to oral, parenteral, orthotopic, intradermal, subcutaneous, intramuscular, intraperitoneal, intranasal, intratumoral, or intravenous injection. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, or about 25% to about 70%. In some embodiments, the compositions are administered orally.

Typically, compositions are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of a pharmaceutical composition are applicable. These are believed to include oral application on a solid physiologically acceptable base or in a physiologically acceptable dispersion, parenterally, by injection and the like. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2 day to twelve week intervals, more usually from one to two week intervals. The course of the administrations may be followed by assays for NF-κB activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The NF-κB signaling pathwayGPR132 inhibitors can be formulated for parenteral administration, e.g., formulated for injection via the intravenous, intradermal, intramuscular, sub-cutaneous, or even intraperitoneal routes. In some embodiments, the composition is administered by intravenous injection. The preparation of an aqueous composition that contains an active ingredient will be known to those of skill in the art in light of the current disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active ingredients in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of an the condition have disappeared or been reduced or after 6, 12, 18, or 24 hours or 1, 2, 3, 4, or 5 days after symptoms of an infection have disappeared or been reduced.

### VIII. COMBINATION THERAPY

The compositions and related methods, particularly administration of a GPR132 inhibitor may also be used in combination with the administration of conventional cancer therapies, such as those known in the art and/or described below.

Conventional cancer therapies include one or more selected from the group of chemical or radiation based treatments and surgery. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein transferase inhibitors, transplatinum, 5-fluorouracil, vincristin, vinblastin and methotrexate, or any analog or derivative variant of the foregoing.

Suitable therapeutic agents include, for example, vinca alkaloids, agents that disrupt microtubule formation (such as colchicines and its derivatives), anti-angiogenic agents, therapeutic antibodies, tyrosine kinase targeting agent (such as tyrosine kinase inhibitors), serine kinase targeting agents, transitional metal complexes, proteasome inhibitors, antimetabolites (such as nucleoside analogs), alkylating agents, platinum-based agents, anthracycline antibiotics, topoisomerase inhibitors, macrolides, therapeutic antibodies, retinoids (such as all-trans retinoic acids or a derivatives thereof); geldanamycin or a derivative thereof (such as 17-AAG), and other standard chemotherapeutic agents well recognized in the art.

In some embodiments, the chemotherapeutic is capecitabine, carboplatin, cyclophosphamide (Cytoxan), daunorubicin, docetaxel (Taxotere), doxorubicin (Adriamycin), epirubicin (Ellence), fluorouracil (also called 5-fluorouracil or 5-FU), gemcitabine, eribulin, ixabepilone, methotrexate, mitomycin C, mitoxantrone, paclitaxel (Taxol), thiotepa, vincristine, and/or vinorelbine.

In some embodiments, the chemotherapeutic agent is any of (and in some embodiments selected from the group consisting of adriamycin, colchicine, cyclophosphamide, actinomycin, bleomycin, daunorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Herceptin®, vinorelbine, capecitabine, Gleevec®, Alimta®, Avastin®, Velcade®, Tarceva®, Neulasta®, Lapatinib, STI-571, ZD1839, Iressa® (gefitinib), SH268, genistein, CEP2563, SU6668, SU11248, EMD121974, and Sorafenib.

Suitable doses for cancer therapeutics in patients include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is used in some embodiments. The drug also sometimes is administered intramuscularly, by infiltration or into body cavities.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

Actual dosage levels of the active ingredients in the methods of this disclosure may be varied so as to obtain an amount of the active ingredient which is effective to achieve the desired therapeutic response for a particular patient, composition and mode of administration, without being toxic to the patient. The selected dosage level will depend upon a variety of factors, including the activity of the chemotherapeutic agent selected, the route of administration, the time of administration, the rate of excretion of the chemotherapeutic agent, the duration of the treatment, other drugs, compounds and/or materials used in combination with the particular chemotherapeutic agent, the age, sex, weight, condition, general health and prior medical history of the patient being treated, and like factors well known in the medical arts.

Administration of pharmaceutical compositions to a patient/subject will follow general protocols for the administration of such compounds, taking into account the toxicity, if any. It is expected that the treatment cycles would be repeated as necessary.

Various combinations with the GPR132 inhibitor and a traditional therapy may be employed, for example, a GPR132 inhibitor is “A” and the traditional therapy (or a combination of such therapies) given as part of a treatment for pancreatitis, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of pharmaceutical compositions to a patient/subject will follow general protocols for the administration of such compounds, taking into account the toxicity, if any. It is expected that the treatment cycles would be repeated as necessary.

### IX. SAMPLES

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from cancer tissue by any of the biopsy methods previously mentioned. In other embodiments the sample may be obtained from any of the tissues provided herein that include but are not limited to gall bladder, skin, heart, lung, breast, pancreas, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects the sample is obtained from cystic fluid or fluid derived from a tumor or neoplasm. In yet other embodiments the cyst, tumor or neoplasm is breast cells. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, scraping, phlebotomy, or any other methods known in the art. In some cases, the sample may be obtained, stored, or transported using components of a kit of the present methods. In some cases, multiple samples, such as multiple cancer samples may be obtained for diagnosis by the methods described herein. In other cases, multiple samples, such as one or more samples from one tissue type and one or more samples from another tissue (for example buccal) may be obtained for diagnosis by the methods. In some cases, multiple samples such as one or more samples from one tissue type (e.g., rectal) and one or more samples from another tissue (e.g., cecum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a tumor or a suspected tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### X. NUCLEIC ACID ASSAYS

Aspects of the methods include assaying nucleic acids to determine expression levels. Arrays can be used to detect differences between two samples. Specifically contemplated applications include identifying and/or quantifying differences between GPR132 from a sample that is normal and from a sample that is not normal, between a cancerous condition and a non-cancerous condition, or between two differently treated samples. Also, GPR132 expression levels may be compared between the expression level of the test sample and a reference level indicating favorable prognosis or poor prognosis.

A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells or include traits of poor prognosis after certain treatment.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze GPR132, their activities, and their effects. Such assays include, but are not limited to, nucleic amplification, polymerase chain reaction, quantitative PCR, RT-PCR, in situ hybridization, digital PCR, dd PCR (digital droplet PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### XI. PROTEIN EXPRESSION ASSAYS

In some embodiments, the gene or protein expression of GPR132 is compared to a control or a reference level. Such methods, like the methods of detecting expression described herein, are useful in providing risk prediction, diagnosis, prognosis, etc., of a disease or cancer.

Methods for measuring transcription and/or translation of a particular gene sequence or biomarker are well known in the art. See, for example, Ausubel, Current Protocols in Molecular Biology, 1987-2006, John Wiley & Sons; and Sambrook and Russell, Molecular Cloning: A Laboratory Manual, 3rd Edition, 2000.

Polypeptides encoded by the GPR132 gene described herein can be detected and/or quantified by any methods known to those of skill in the art from samples as described herein. In some embodiments, antibodies can also be used to detect polypeptides encoded by the genes described herein. Antibodies to these polypeptides can be produced using well known techniques (see, e.g., Harlow & Lane, 1988 and Harlow & Lane, 1999; Coligan, 1991; Goding, 1986; and Kohler & Milstein, 1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., 1989; Ward et al., 1989).

Once specific antibodies are available, GPR132 expression can be detected by a variety of immunoassay methods. For a review of immunological and immunoassay procedures, see Basic and Clinical Immunology (1991). Moreover, the immunoassays of certain aspects can be performed in any of several configurations, which are reviewed extensively in Enzyme Immunoassay (1980); and Harlow & Lane, supra).

Immunoassays also often use a labeling agent to specifically bind to and label the complex formed by the antibody and antigen. The labeling agent may itself be one of the moieties comprising the antibody/antigen complex. Thus, the labeling agent may be a labeled polypeptide or a labeled antibody that binds the protein of interest. Alternatively, the labeling agent may be a third moiety, such as a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the labeling agent. These proteins exhibit a strong non-immunogenic reactivity with immunoglobulin constant regions from a variety of species (see, e.g., Kronval et al., 1973; Akerstrom et al., 1985). The labeling agent can be modified with a detectable moiety, such as biotin, to which another molecule can specifically bind, such as streptavidin. A variety of detectable moieties are well known to those skilled in the art.

Commonly used assays include noncompetitive assays, e.g., sandwich assays, and competitive assays. In competitive assays, the amount of polypeptide present in the sample is measured indirectly by measuring the amount of a known, added (exogenous) polypeptide of interest displaced (competed away) from an antibody that binds by the unknown polypeptide present in a sample. Commonly used assay formats include immunoblots, which are used to detect and quantify the presence of protein in a sample. Other assay formats include liposome immunoassays (LIA), which use liposomes designed to bind specific molecules (e.g., antibodies) and release encapsulated reagents or markers. The released chemicals are then detected according to standard techniques (see Monroe et al., 1986).

Any suitable method can be used to detect one or more of the markers described herein. Successful practice can be achieved with one or a combination of methods that can detect and, preferably, quantify the markers. These methods include, without limitation, hybridization-based methods, including those employed in biochip arrays, mass spectrometry (e.g., laser desorption/ionization mass spectrometry), fluorescence (e.g., sandwich immunoassay), surface plasmon resonance, ellipsometry and atomic force microscopy. Expression levels of markers (e.g., polynucleotides or polypeptides) are compared by procedures well known in the art, such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays. Methods may further include, one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)11, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry, where n is an integer greater than zero.

Detection methods may include the use of a biochip array. Biochip arrays include protein and polynucleotide arrays. The protein of interest may be captured on the biochip array and subjected to analysis to detect the level of the protein in a sample.

### XII. KITS

Certain aspects concern kits containing compositions described herein or compositions to implement methods described herein.

In various aspects, a kit is envisioned containing therapeutic agents and/or other therapeutic and delivery agents. In some embodiments, a kit for preparing and/or administering a therapy described herein may be provided. The kit may comprise one or more sealed vials containing any of the pharmaceutical compositions, therapeutic agents and/or other therapeutic and delivery agents. In some embodiments, the lipid is in one vial, and the therapeutic agent is in a separate vial. The kit may include, for example, at least one inhibitor of GPR132 expression/activity, one or more lipid component, as well as reagents to prepare, formulate, and/or administer the components described herein or perform one or more steps of the methods. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of a therapeutic agent.

In some embodiments, kits may be provided to evaluate the expression of GPR132 or related molecules. Such kits can be prepared from readily available materials and reagents. For example, such kits can comprise any one or more of the following materials: enzymes, reaction tubes, buffers, detergent, primers and probes, nucleic acid amplification, and/or hybridization agents. In a particular embodiment, these kits allow a practitioner to obtain samples in blood, tears, semen, saliva, urine, tissue, serum, stool, colon, rectum, sputum, cerebrospinal fluid and supernatant from cell lysate. In another embodiment, these kits include the needed apparatus for performing RNA extraction, RT-PCR, and gel electrophoresis. Instructions for performing the assays can also be included in the kits.

Kits may comprise components, which may be individually packaged or placed in a container, such as a tube, bottle, vial, syringe, or other suitable container means. The components may include probes, primers, antibodies, arrays, negative and/or positive controls. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

The kit can further comprise reagents for labeling GPR132 in the sample. The kit may also include labeling reagents, including at least one of amine-modified nucleotide, poly(A) polymerase, and poly(A) polymerase buffer. Labeling reagents can include an amine-reactive dye or any dye known in the art.

The components of the kits may be packaged either in aqueous media or in lyophilized form. The container means of the kits will generally include at least one vial, test tube, flask, bottle, syringe or other container means, into which a component may be placed, and preferably, suitably aliquotted. Where there is more than one component in the kit (labeling reagent and label may be packaged together), the kit also will generally contain a second, third or other additional container into which the additional components may be separately placed. However, various combinations of components may be comprised in a vial. The kits may also include a means for containing the nucleic acids, antibodies or any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

Alternatively, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits in certain aspects. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits may include a means for containing the vials in close confinement for commercial sale, such as, e.g., injection and/or blow-molded plastic containers into which the desired vials are retained.

A kit may also include instructions for employing the kit components as well the use of any other reagent not included in the kit. Instructions may include variations that can be implemented.

### XIII. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of particular embodiments, are exemplary, and are not intended as limitations on the scope of the invention. Changes therein and other uses which are encompassed within the spirit of the invention as defined by the scope of the claims will occur to those skilled in the art.

### Example 1: Macrophage PPARΓ Deletion Enhances Tumor Growth In Vivo

Macrophage PPARγ knockout mice (mf-g-KO) were generated by breeding PPARγ fox mice with Tie2Cre or Lysozyme-Cre (LyzCre). Tie2Cre deleted PPARγ in hematopoietic cells and endothelial cells were made as previously described (Wan et al., 2007a; Wan et al., 2007b). LyzCre deleted PPARγ specifically in the myeloid lineage was made as previously described (Clausen et al., 1999). PPARγ^{flox/flox}; Cre^{+/-} KO mice were compared with PPARγ^{flox/flox}; Cre^{-/-} littermate controls.

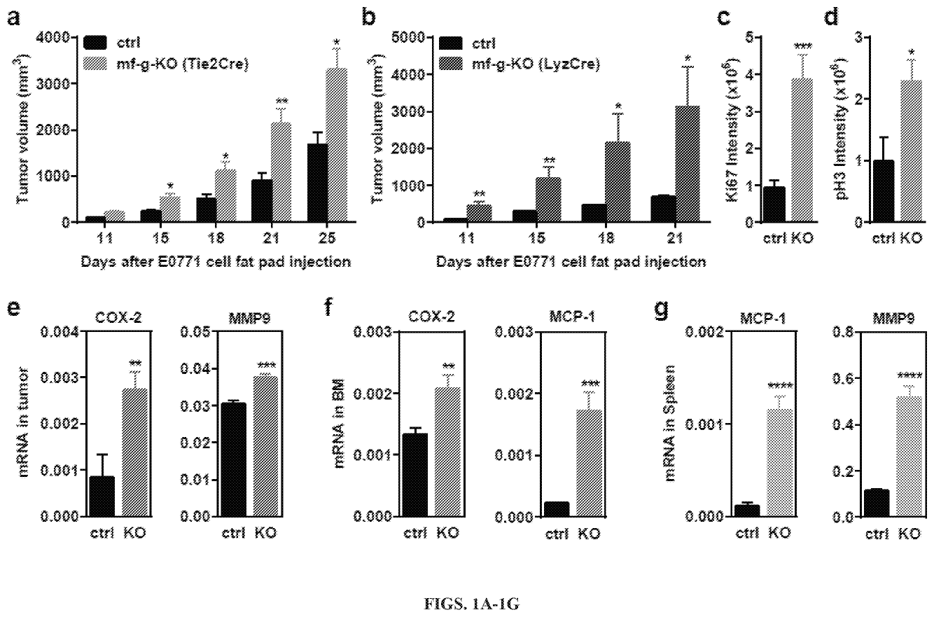

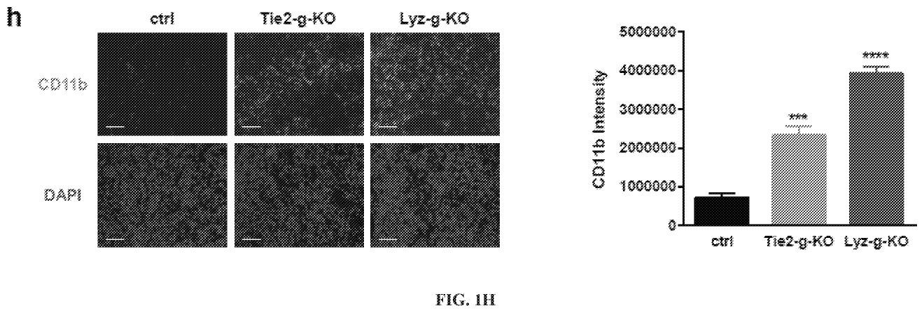

To determine the effects of macrophage PPARγ deletion on breast cancer development, mammary fat pad orthotopic injections of C57BL/6J-compatible mouse breast cancer cells EO771 was performed in female mice, and tumor growth was followed by measuring tumor size. Compared to the littermate controls, both Tie2Cre-induced and LyzCre-induced mf-g-KO mice showed enhanced tumor development as indicated by earlier onset and larger tumor volume (FIG. 1A-B). These results indicate that the pro-tumor effect observed was largely caused by PPARγ deletion in myeloid cells such as macrophages. Staining for Ki67 and phospho histone H3 (PH3) in the tumor sections showed increased cell proliferation in mf-g-KO mice (FIG. 1C-D). These findings suggest that macrophage PPARγ inhibits tumor growth in vivo.

### Example 2: Macrophage PPARγ Deletion Increased Tam Abundance In Vivo

Tumor tissues, bone marrow cells and spleen cells were collected from tumor-bearing mf-g-KO or control mice, and gene expression was compared. The results showed a higher expression of pro-inflammatory genes in these PPARγ-deficient cells and tissues, including COX-2, MMP9 and MCP-1 (FIG. 1E-G). Macrophage infiltration into tumors is a strong indicator for cancer malignancy and poor prognosis (Komohara et al., 2014; Ruffell and Coussens, 2015; Zhang et al., 2012). Immunofluorescence staining using CD11b and F4/80 markers revealed enhanced TAM recruitment in both Tie2-g-KO and Lyz-g-KO mice compared with control mice (FIG. 1H, 6A-B). Consistent with the reports that PPARγ agonists inhibit angiogenesis (Goetze et al., 2002; Keshamouni et al., 2005; Scoditti et al., 2010), it was found that the number of blood vessels in tumor sections was increased in Tie2-g-KO mice but unaltered in Lyz-g-KO mice (FIG. 6C-D), further indicating that PPARγ deficiency in macrophage alone is sufficient to augment tumor growth independent of changes in angiogenesis. Together, these findings suggest that macrophage PPARγ deletion changes both the number and property of TAMs to establish a pro-inflammatory tumor environment.

### Example 3: PPARγ-Deficient Macrophages Promote Cancer Cell Proliferation In Vitro

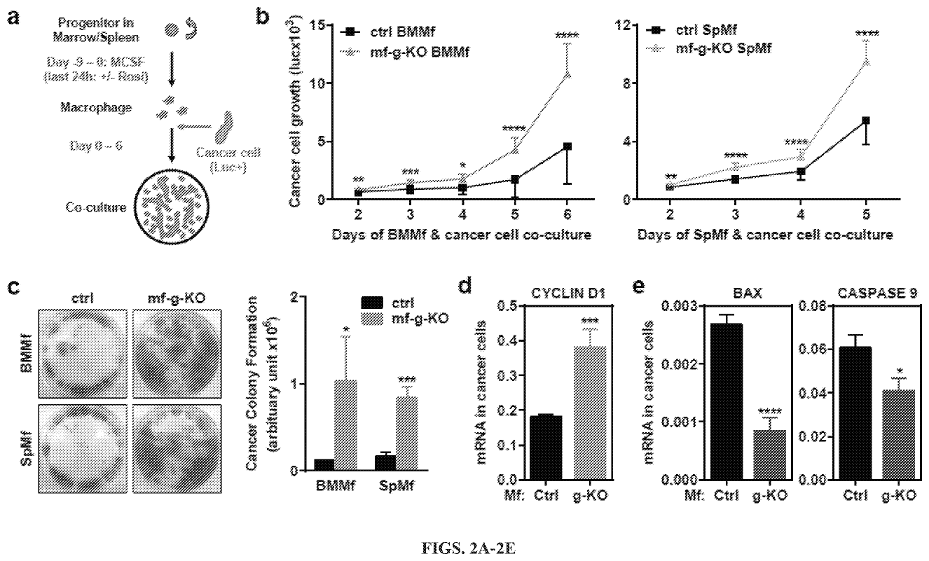

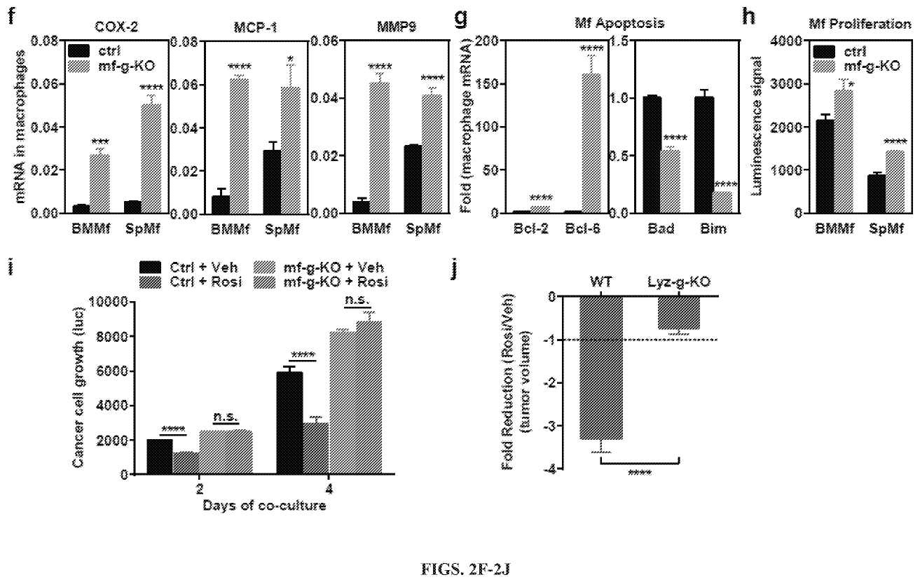

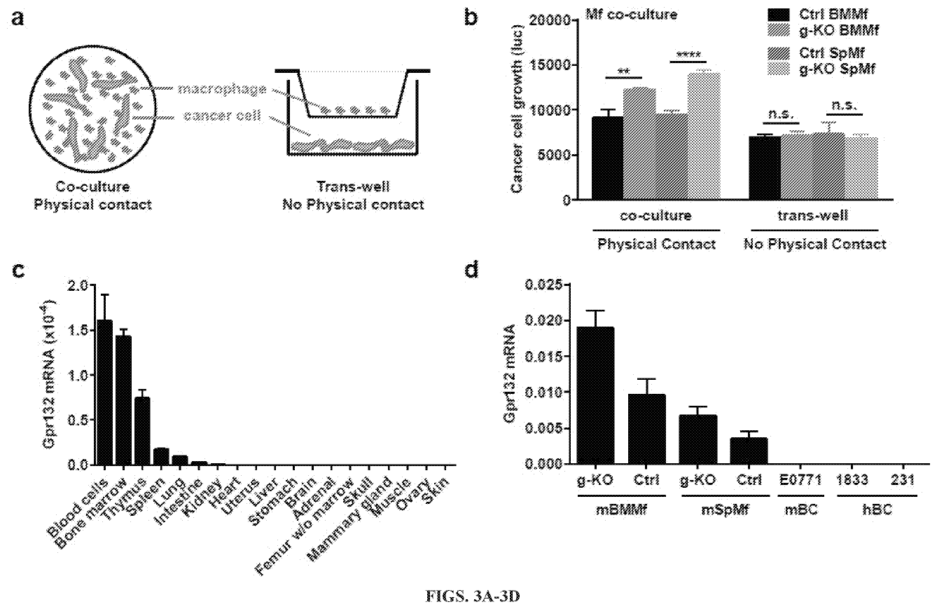

To determine if PPARγ-deficient macrophages regulate cancer cell behavior in the absence of other components in the tumor microenvironment such as fibroblasts and extracellular matrix, macrophage and cancer cell co-culture experiments in vitro were performed (FIG. 2A). Mouse macrophages were differentiated from the progenitors in bone marrow or spleen and then co-cultured with a luciferase-labelled subline of the MDA-MB-231 human breast cancer cell line (1833 cells). Specific quantification of tumor cell proliferation was achieved by luciferase output as only the cancer cells, but not the macrophages, were tagged with a luciferase reporter. The results showed that tumor cell proliferation was significantly augmented by PPARγ-deficient macrophages compared with WT control macrophages (FIG. 2B). Consistent with this observation, co-culture with PPARγ-deficient macrophages also led to an increased tumor cell colony formation (FIG. 2C). Since mouse macrophages and human cancer cells were from different species, mRNA expression in these two cell types in the co-culture setting could be distinguished by species-specific QPCR primers. It was found that co-culture with PPARγ-deficient macrophages resulted in higher expression of proliferation markers and lower expression of apoptosis markers in cancer cells compared with WT control macrophages (FIG. 2D-E).

In accordance to the in vivo observations (FIG. 1), PPARγ-deficient macrophages exhibited elevated expression of pro-inflammatory genes such as COX-2, MCP-1 and MMP-9 (FIG. 2F). In addition, PPARγ-deficient macrophages displayed higher levels of anti-apoptotic genes and lower levels of pro-apoptotic genes (FIG. 2G), indicating an augmented survival. Moreover, PPARγ-deficient macrophages showed increased proliferation, measured by ATP content (FIG. 2H) or MTT assay (not shown). The in vitro findings further support the in vivo observations that the increased number and pro-inflammatory property of PPARγ-deficient macrophages are sufficient to promote tumor progression.

### Example 4: Rosiglitazone Activation of Macrophage PPARγ Inhibits Cancer Cell Proliferation In Vitro

As a complementary approach to the loss-of-function genetic approach, gain-of-function pharmacological experiment was performed to assess the effect of rosiglitazone activation of macrophage PPARγ on cancer cells. Mouse macrophages were pre-treated with rosiglitazone or vehicle control; rosiglitazone was removed by medium change before human cancer cells were seeded for co-culture (FIG. 2A). The results showed that cancer cell growth was significantly inhibited when co-cultured with rosiglitazone-treated WT macrophages compared with vehicle-treated WT macrophages (FIG. 2I). Importantly, this rosiglitazone effect was macrophage-PPARγ-dependent because tumor cell proliferation was increased equally when co-cultured with PPARγ-deficient macrophages regardless of rosiglitazone or vehicle treatment (FIG. 2I). Together, these findings indicate that activation of macrophage PPARγ by either endogenous or synthetic agonists suppresses tumor growth.

### Example 5: Macrophage PPARγ is a Key Mediator of the Anti-Tumor Effect of Rosiglitazone In Vivo

To assess the functional significance of macrophage PPARγ in the pharmacological effects of rosiglitazone, mf-g-KO mice and littermate controls were treated with rosiglitazone or vehicle control starting 4 days after cancer cell injection. The results show that the ability of rosiglitazone to suppress breast cancer growth was significantly attenuated in mf-g-KO mice (FIG. 2J). This indicates that macrophage is an essential cell type that is required for the anti-tumor function of rosiglitazone.

### Example 6: Macrophage PPARγ Represses Gpr132 Expression

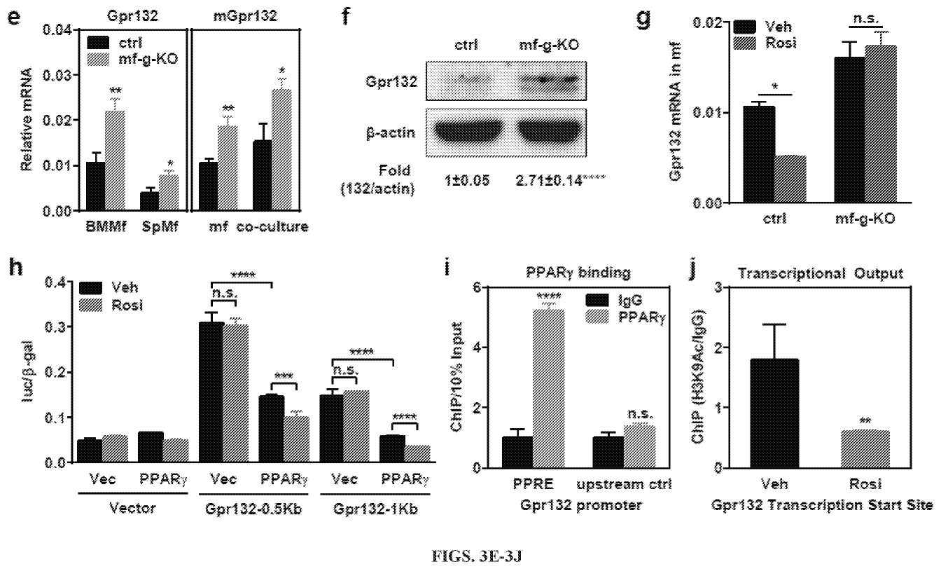

To understand how PPARγ alters the transcription program in macrophages to control cancer cell proliferation, the inventors next set out to identify the key PPARγ target genes. The experiments reveal that tumor cell proliferation could be significantly enhanced by co-culture with PPARγ-deficient macrophages but not by the conditioned medium from PPARγ-deficient macrophages (FIG. 3A-B), indicating that physical contact between macrophages and cancer cells is required and thus the key tumor-modulating PPARγ target gene in macrophages likely encodes a membrane protein. By searching published microarray databases (Hevener et al., 2007; Welch et al., 2003), several candidate membrane proteins that might be regulated by PPARγ in macrophages were selected. Upon examining their expression in the macrophage cultures, it was found that G protein-coupled receptor 132 (GPR132, also known as G2A) was consistently and significantly upregulated in PPARγ-deficient macrophages compared with WT control macrophages (see below), whereas the expression of 11 other candidates was unaltered (FIG. 7). Therefore, the inventors decided to further investigate whether GPR132 is a functional PPARγ target gene in macrophages.

GPR132 has been previously described as a stress-inducible seven-pass transmembrane receptor that functions at the G2/M checkpoint of the cell cycle (Weng et al., 1998), which modulates immune cell function (Kabarowski, 2009; Radu et al., 2004; Yang et al., 2005). The inventors found that GPR132 was predominantly expressed in the hematopoietic cell types/tissues and highly expressed in macrophages, but largely absent in other tissues or tumor cells (FIG. 3C-D), indicating that it may play an important role in macrophage function. GPR132 expression was significantly higher in PPARγ-deficient macrophages compared with control macrophages, either in macrophage cultures alone or in macrophages co-cultured with cancer cells (FIG. 3E-F). In line with this observation, PPARγ activation by rosiglitazone reduced GPR132 expression in WT macrophages but not PPARγ-deficient macrophages (FIG. 3G). These findings suggest that PPARγ represses GPR132 expression.

### Example 7: PPARγ Binds to Gpr132 Promoter and Represses its Transcriptional Activity

To determine whether GPR132 is a direct PPARγ transcriptional target, the inventors investigated whether PPARγ can bind to the GPR132 promoter and regulate its transcription. GPR132 promoter regions (0.5 kb and 1 kb) were cloned into a luciferase reporter vector. Transient transfection and reporter assays reveal that luciferase output from both 0.5 Kb and 1 Kb GPR132 promoter was reduced by the co-transfection of PPARγ and further diminished by rosiglitazone treatment (FIG. 3H). These results indicate that PPARγ represses GPR132 promoter via critical element(s) within the 500 base pairs upstream of GPR132 transcription start site. Indeed, the inventors identified a PPAR response element (PPRE) half site in this region (−188: CATCCGAGCAAGGTCAGAC (SEQ ID NO:2)). Chromatin-immunoprecipitation (ChIP) assay showed that PPARγ could bind to the endogenous GPR132 proximal promoter in macrophages but not an upstream negative control region (FIG. 3I); moreover, rosiglitazone treatment led to a decreased level of H3K9Ac active transcription histone mark at the GPR132 transcriptional start site (FIG. 3J). These mechanistic studies reveal that PPARγ directly represses GPR132 transcription in macrophages.

### Example 8: Gpr132 is Repressed by PPARγ in Human Macrophages and Correlates with Human Breast Cancer

GPR132 expression in human macrophages derived from human peripheral blood mononuclear cells (hPBMN) was also blunted by rosiglitazone (FIG. 4A). This indicates that PPARγ repression of GPR132 is evolutionally conserved and the inventors' findings in mice may translate to human physiology and disease. To explore the significance of GPR132 in human breast cancer, the RNA-Seq and clinical data of breast invasive carcinoma (BRCA) from The Cancer Genome Atlas (TCGA) database was analyzed. Because GPR132 is highly expressed in human macrophages (FIG. 4A) but absent in human breast cancer cells (FIG. 3D), GPR132 expression in tumors mainly originates from hematopoietic cells in the microenvironment such as macrophages. Compared with normal breast samples, the majority of breast cancer lesions displayed significantly higher GPR132 expression (FIG. 4B); in addition, compared with ER-positive breast cancers, the more aggressive ER-negative breast cancers also exhibited higher GPR132 expression (FIG. 4C). Immunohistochemistry staining confirmed that human breast cancer tissues expressed significantly higher GPR132 compared with normal breast control tissues (FIG. 4D-E). Moreover, linear regression analyses showed that higher GPR132 expression was significantly correlated with higher expression of pro-inflammatory markers including CCL2 (MCP-1), MMP9 and PTGS2 (COX-2) in breast cancer lesions (FIG. 4F). These findings further suggest that macrophage GPR132 may promote inflammation and tumor progression.

### Example 9: Macrophage Gpr132 Facilitates Cancer Cell Proliferation In Vitro

The inventors next examined the function of macrophage GPR132 in regulating cancer cells using the in vitro co-culture system. GPR132 knockdown in macrophages significantly reduced cancer cell growth (FIG. 5A-C). Conversely, GPR132 over-expression in macrophages increased cancer cell growth (FIG. 5D-F). The inventors then compared macrophages derived from the bone marrow or spleen of GPR132-KO mice vs. littermate WT control mice. Gene expression analyses reveal that GPR132-KO macrophages displayed the opposite phenotype from PPARγ-deficient macrophages, with lower pro-inflammatory genes (FIG. 5G), higher pro-apoptotic genes and lower anti-apoptotic genes (FIG. 511) compared with WT macrophages. In vitro macrophage-tumor cell co-culture experiments showed that GPR132-KO macrophages exhibited a significantly reduced ability to promote cancer cell colony formation and growth (FIG. 5I-J). These results indicate that GPR132 enhances inflammation and macrophage survival, and the upregulated GPR132 in PPARγ-deficient macrophages may confer their tumor-promoting effects.

### Example 10: Gpr132 Knockout Mice Support Less Tumor Growth In Vivo

To examine the effects of GPR132 deletion in the tumor environment on cancer growth in vivo, EO771 mouse breast cancer cells were injected into the mammary fat pad of GPR132-KO mice and WT littermate controls. In this system, GPR132 was deleted in macrophages as well as other GPR132-expressing tissues such as bone marrow, spleen and thymus, but not in the injected cancer cells which had essentially no GPR132 expression (FIG. 3C-D). Previous study show that GPR132-KO mice display a normal pattern of T and B lineage differentiation, appearing healthy and indistinguishable from WT littermates throughout young adulthood, but develop progressive secondary lymphoid organ enlargement associated with abnormal expansion of both T and B lymphocytes that become pathological when older than one year of age (Le et al., 2001). Therefore, these experiments were initiated in young mice and terminated before GPR132-KO mice aged to prevent any potential effects of lymphoid defects on cancer growth. Compared with WT and GPR132 heterozygous (Het) controls, GPR132-KO mice exhibited significantly diminished tumor growth (FIG. 5K). Compared with WT controls, GPR132-Het mice also showed attenuated tumor growth at later stage (FIG. 5K), indicating that GPR132 regulation is dosage-sensitive. Together, these in vitro and in vivo results indicate that macrophage GPR132 promotes tumor growth, suggesting that GPR132 inhibition may impede cancer progression.

### Example 11: Macrophage Gpr132 Mediates PPARγ Regulation and Rosiglitazone Effects

To further examine whether GPR132 is a functional PPARγ target in macrophage that is required for PPARγ cancer regulation, the inventors conducted pharmacological and genetic experiments. As a pharmacological gain-of-function strategy, the inventors treated the macrophage-cancer cell co-cultures or tumor-grafted mice with rosiglitazone or vehicle control. Pre-treating GPR132-KO macrophages with rosiglitazone before cancer cell seeding no longer showed any inhibition of cancer cell proliferation in the co-cultures (FIG. 5L, 8). Consistent with this in vitro observation, the anti-tumor effect of rosiglitazone in vivo was also abolished in GPR132-KO mice (FIG. 5M). These pharmacological findings support that PPARγ repression of GPR132 in macrophages is a significant contributor to the anti-tumor effects of rosiglitazone.

As a genetic loss-of-function strategy, GPR132-KO mice were bred with mf-g-KO mice to generate mf-g/GPR132 double KO (DKO) mice. Mammary fat pad tumor graft experiments demonstrated that GPR132 deletion in the DKO mice impaired the ability of macrophage PPARγ deficiency to exacerbate tumor growth because DKO mice showed similar tumor volume as GPR132-KO mice (FIG. 5N). This genetic rescue further supports that GPR132 is an essential mediator of macrophage PPARγ regulation of breast cancer progression.

### Example 12: Pharmacological Gpr132 Inhibition Impedes Tumor Growth

To further explore GPR132 as a potential cancer therapeutic target, the inventors next examined whether acute pharmacological inhibition of GPR132 could attenuate breast cancer progression. Because macrophage precursors reside in hematopoietic tissues such as blood, bone marrow and spleen that can be efficiently targeted by siRNA (Larson et al., 2007), siRNA-mediated GPR132 knockdown was employed. WT female mice were treated with si-GPR132 or si-Ctrl for 18 days via intravenous injection at 10m/mouse twice/week, 3 days before and 15 days after cancer cell graft. The results showed that si-GPR132 significantly reduced tumor volume compared with si-Ctrl (FIG. 5O), as the result of depleted GPR132 expression (FIG. 5P). Body weight was unaltered by si-GPR132 (not shown), indicating a lack of overt toxicity by GPR132 inhibition. These results support GPR132 inhibition as a novel anti-cancer strategy.

### Example 13: Tumor-Derived Factors Activate M2-Like Macrophages Via Gpr132

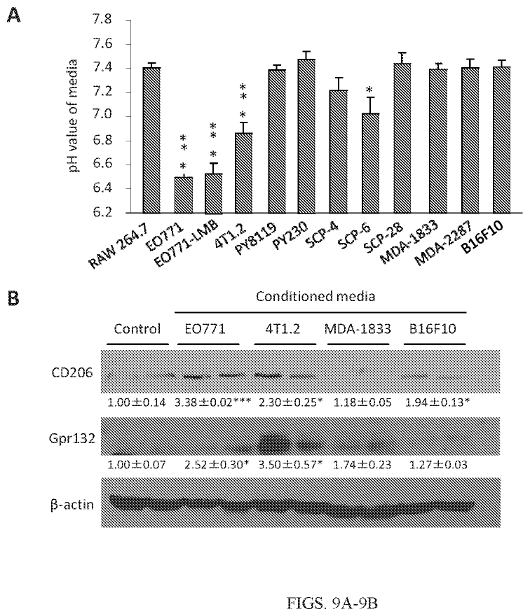

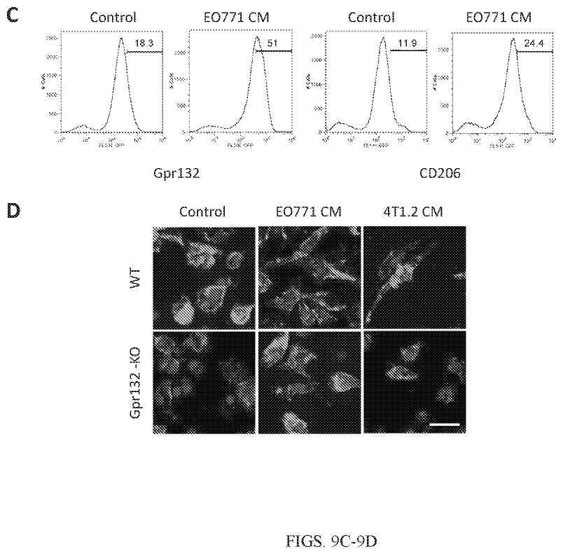

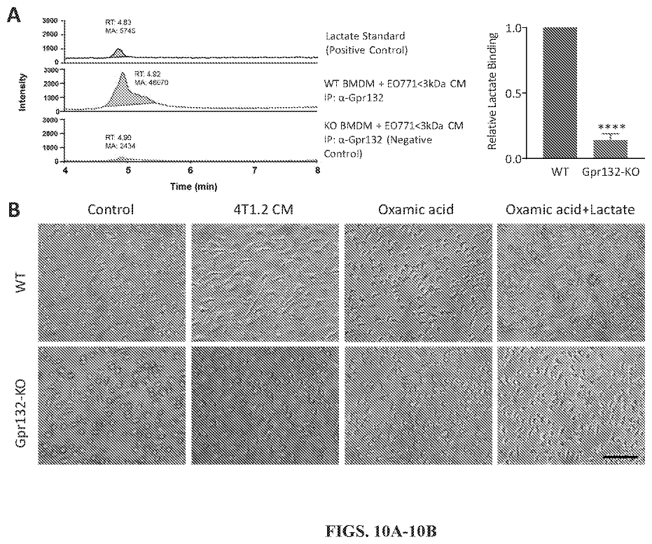

Previous studies have demonstrated that macrophages in the tumor microenvironment are educated by cancer cells. Gpr132 is a cell surface receptor highly expressed in macrophages but largely absent from breast cancer cells (data not shown). At the same time, Gpr132 is also highly sensitive to acidity—a hallmark of cancer milieu. Thus, it was hypothesized that Gpr132 might be the macrophage pH sensor that controls the macrophage phenotype in response to the acidic tumor microenvironment. To examine whether cancer cell-derived acidic signals can modulate macrophage M2 activation and Gpr132 expression, the pH value in the conditioned medium (CM) from 10 equally seeded breast cancer cell lines was first measured, as well as B 16F10 melanoma and RAW 264.7 macrophage cell lines. It was found that the pH values of breast cancer cell CM from EO771, EO771-LMB, 4T1.2 and SCP-6 cell lines were significantly lower than macrophage CM (FIG. 9A). Western blot RT-quantitative PCR (qPCR), and flow cytometry analyses of RAW264.7 macrophage cell line and bone marrow derived macrophages (BMDMs) showed that EO771 CM or 4T1.2 CM significantly enhanced the expression of Gpr132 and CD206 (mannose receptor, a M2 macrophage marker) (FIGS. 9B-C, 15A, 25, and 26). These results suggest that the acidic signals in EO771 CM and 4T1.2 CM may facilitate macrophage M2 activation via Gpr132.

Bone marrow cells from wild-type (WT) or Gpr132 knockout (Gpr132-KO) mice were differentiated into macrophages with or without 30% (vol/vol) EO771 CM or 4T1.2 CM for 7 days. BMDMs from WT mice but not Gpr132-KO mice, when treated with EO771 CM or 4T1.2 CM, became elongated and stretched, a feature of M2-like TAMs (FIG. 9D). Moreover, EO771 CM or 4T1.2 CM enhanced the expression of M2 markers, such as arginase 1 (Arg-1) and CD206, in WT BMDMs but not or to a lesser extent in Gpr132-KO BMDMs (FIG. 15B, 16C). These results indicate a key role of Gpr132 in macrophage M2 activation upon education by cancer cell acidic signals.

### Example 14: Tumor-Derived Lactate Stimulates Gpr132 to Promote Macrophage M2 Activation

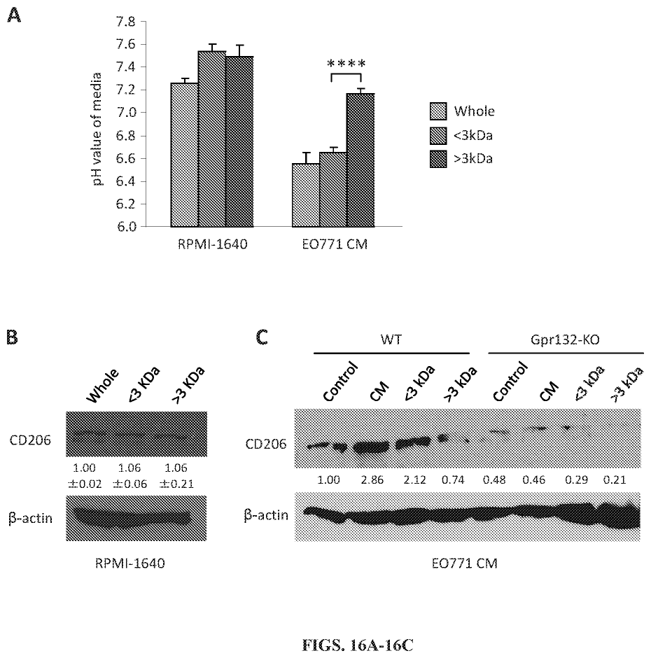

Considering that Gpr132 is an acidic signal-sensing receptor, and the reported Gpr132 ligands such as 9-Hydroxyoctadecadienoic acid (20) are small molecules EO771 CM was fractionated by size (<3 kDa and >3 kDa), and then compared the pH value and M2 activation function in both fractions. It was found that the <3 kDa fraction exhibited lower pH than the >3 kDa fraction (FIG. 16A). Moreover, the <3 kDa fraction, but not the >3 kDa fraction, enhanced CD206 expression in WT macrophages; whereas neither fraction had an effect on Gpr132-KO macrophages (FIG. 16C). Fractionation of basal culture media did not change the pH value or CD206 expression in macrophages (FIG. 16A-B). These results suggest that the small molecule soluble factors in the <3 kDa fraction of EO771 CM may function as Gpr132 ligands/activators to promote the macrophage M2-like phenotype.

To test whether lipid factors were involved, lipids from the <3 kDa fraction of EO771 CM were isolated and applied to WT and Gpr132-KO macrophages. These lipids did not enhance M2 phenotype, but instead exhibited slight inhibitory effects in both WT and Gpr132-KO macrophages (FIG. 17A-E). These results exclude the potential role of CM lipids in stimulating M2 macrophages or activating Gpr132, suggesting other factors may be responsible, such as lactate, which is a potent tumor-derived factor inducing TAM polarization.

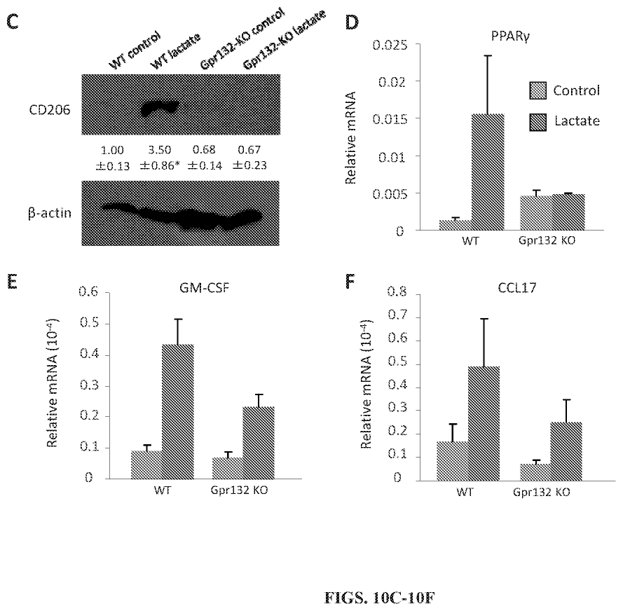

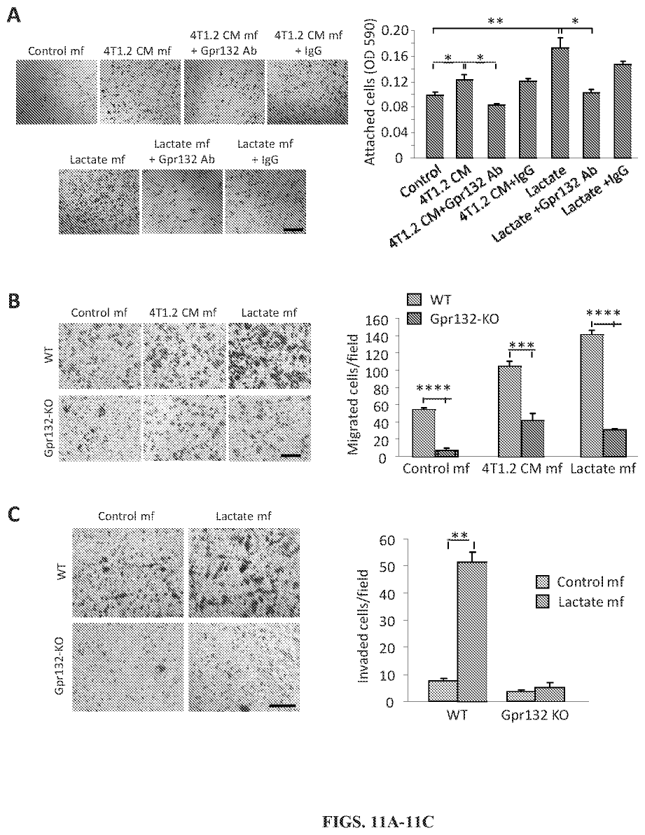

To determine whether lactate in the <3 kDa fraction of EO771 CM could bind to macrophage Gpr132, co-immunoprecipitation with anti-Gpr132 in WT and Gpr132-KO BMDMs was performed. Lactate pulled down by Gpr132 was quantified by liquid chromatography-mass spectrometry (LC-MS). The results showed that lactate was enriched by, 7.1-fold in the eluent from WT macrophages compared with Gpr132-KO macrophages (FIG. 10A), suggesting that lactate is a potential ligand of Gpr132.

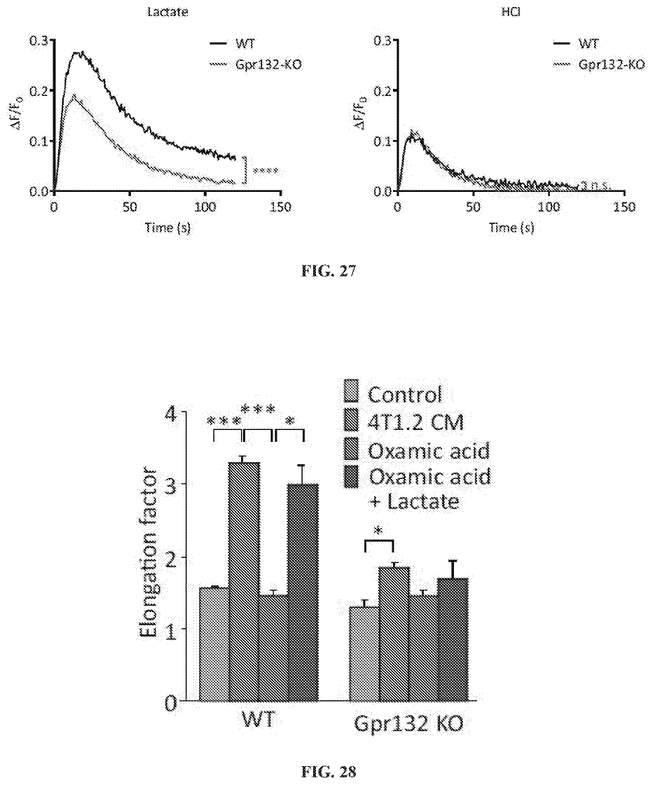

To determine whether Gpr132 is required for lactate signaling in macrophages, a calcium mobilization assay was performed. The results showed that Gpr132 deletion specifically compromised lactate-triggered, but not hydrochloric acid (HCl)-triggered, calcium mobilization (FIG. 27). This finding not only further supports Gpr132 as a functional receptor for lactate but also reveals lactate, rather than simply low pH, as a key activation signal of Gpr132. It was next examined whether lactate was the main factor responsible for the Gpr132-mediated EO771/4T1.2 CM-induced M2 macrophage. First, lactate levels in the CM of distinct cancer cell lines were measured using a Vitros 250 chemistry analyzer. It was found that the lactate level was significantly higher in the lower pH EO771 CM and 4T1.2 CM when compared with CM of other breast cancer cell lines (FIG. 18A). Lactate was secreted from EO771 cells in a time-dependent manner (FIG. 18B), and distributed in the <3 kDa fraction (FIG. 18C). Second, the effects of blocking lactate production from EO771 and 4T1.2 cells by oxamic acid, an inhibitor of lactate dehydrogenase was tested. Oxamic acid treatment depleted lactate in the CM (FIG. 18D), leading to a significant increase of pH (FIG. 18E), indicating that lactic acid was the main contributor of CM acidity. Importantly, oxamic acid treatment completely abolished the ability of the EO771 or 4T1.2 CM to induce M2-like morphology in WT BMDMs (FIG. 10B, 19A), which was restored by the addition of exogenous lactate (FIG. 10B). This finding indicates that lactate was the major mediator of the TAM-modulating activity in the cancer cell CM. Moreover, Gpr132-KO BMDMs were refractory to any of these treatments (FIG. 10B, 19A), further supporting the essential role of Gpr132 in sensing and responding to lactate.

To confirm the role of the lactate-Gpr132 axis in M2 macrophage activation, WT and Gpr132-KO macrophages were treated with exogenous lactate. Western blot and RT-qPCR analyses showed that lactate increased the expression of M2 markers in WT macrophages, including CD206, granulocyte macrophage colony-stimulating factor (GM-CSF) and C-C motif chemokine ligand 17 (CCL17), but these effects were absent or largely attenuated in Gpr132-KO macrophages (FIG. 10C-F, 19B). Taken together, these results suggest that Gpr132 is a macrophage lactate receptor/sensor, and cancer cell-derived lactate is a Gpr132 ligand/activator that stimulates macrophage M2 polarization.

### Example 15: Lactate Activation of Gpr132 Enhances Macrophage AKT/MTOR Signaling

To identify the signaling pathways that may mediate the function of lactate-Gpr132 axis in M2 macrophage activation, PathScan Akt Signaling Antibody Array was used to screen potential targets due to the importance of the Akt pathway in macrophage polarization. It was found that phosphorylation of AKT (Thr308), mammalian target of rapamycin (mTOR, Ser2481), S6 Ribosomal protein (Ser235/236) and RSK-1 (Thr421/Ser424) was increased upon lactate treatment in WT but not Gpr132-KO macrophages (FIG. 14). These findings reveal that lactate activation of Gpr132 facilitates macrophage M2 activation by augmenting AKT/mTOR signaling.

### Example 16: Gpr132 Specifically Respond to Lactate to Activate M2 Macrophages

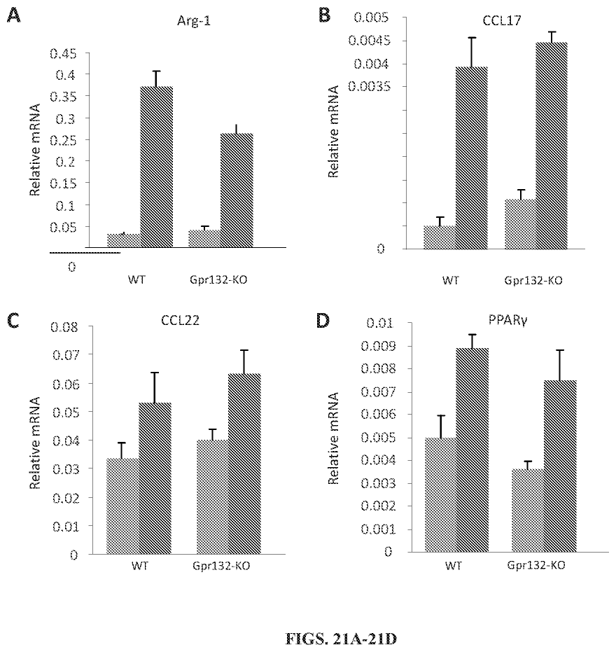

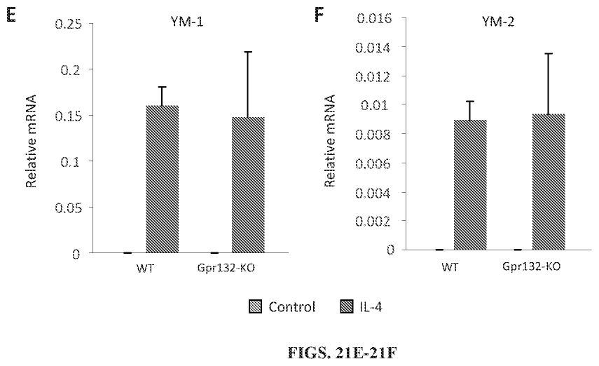

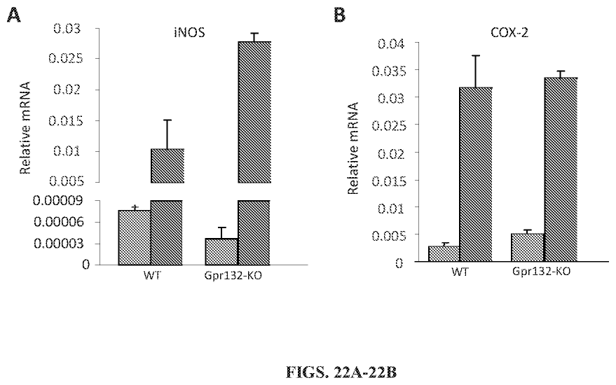

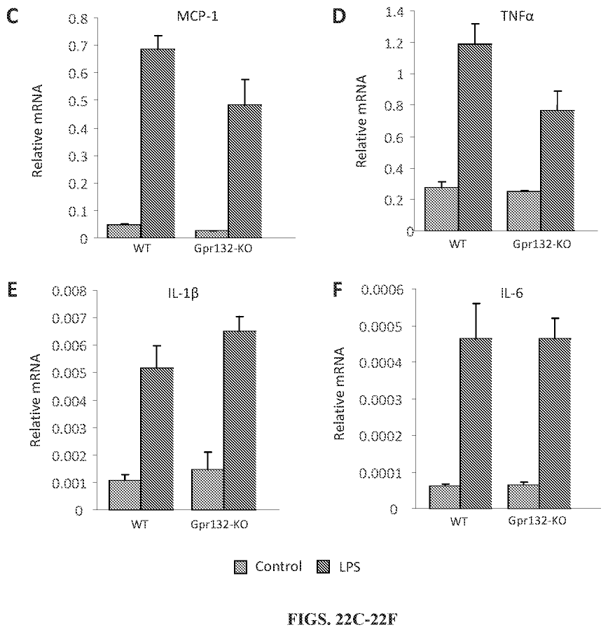

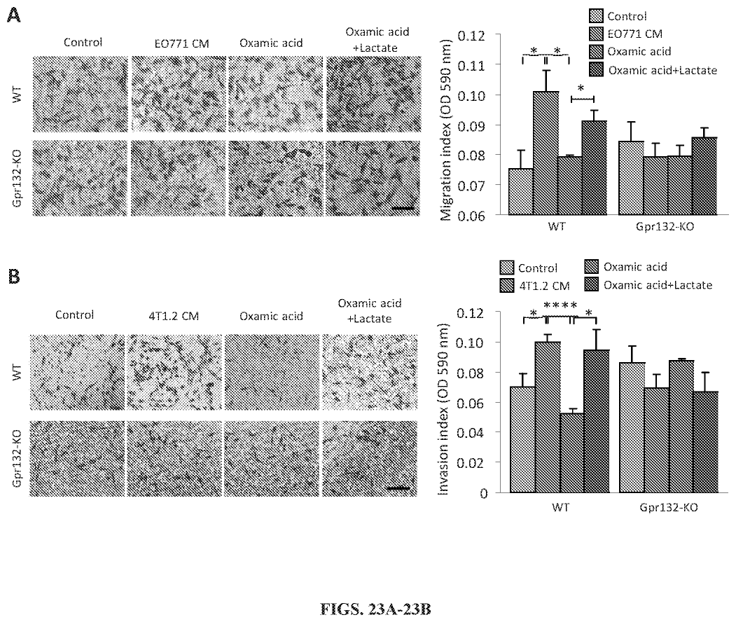

To examine whether Gpr132 is a specific receptor/sensor of lactate during M2 macrophage activation, macrophages were treated with interleukin-4 (IL-4), a notable and standard T helper 2 cytokine widely used to trigger macrophage M2 activation. It was found that WT and Gpr132-KO macrophages responded to IL-4 equally well for the induction of M2 markers, including Arg-1, CCL17, CCL22, peroxisome proliferator-activated receptor gamma (PPAR-y), and chitinase 3-like 3 and 4 (also known as YM-1 and YM-2, respectively) (FIGS. 21A-F and 29). This finding suggests that IL-4 induction of M2 macrophages is independent of Gpr132. Moreover, lipopolysaccharide (LPS)-induced M1 macrophage activation was also largely intact in Gpr132-KO macrophages (FIG. 22A-F). These data indicate that Gpr132 is a specific macrophage receptor/sensor for lactate that specifically mediates lactate-induced M2 macrophage activation.

### Example 17: Lactate-Activated Macrophages Promote Breast Cancer Cell Adhesion, Migration and Invasion Via GPR132 In Vitro

M2 macrophages have been shown to facilitate breast cancer metastasis via secreted factors. Thus, it was investigated whether lactate-induced M2 macrophages promote breast cancer cell adhesion, migration and invasion via paracrine mechanisms in a Gpr132-dependent manner. The effects of the CM from various pre-treated macrophages on breast cancer cell adhesion were first examined. Compared with CM from untreated control macrophages, CM from 4T1.2 CM- and lactate-activated macrophages significantly increased the adherence of 4T1.2 cells to fibronectin (FIG. 11), the most abundant extracellular matrix protein in breast cancer stroma. Interestingly, it was found that these effects were abrogated by a Gpr132 blocking antibody but not an IgG isotype control (FIG. 11A).

Boyden Chamber Assays were next used to examine the migration and invasion of breast cancer cells by plating them in uncoated or matrigel-coated upper inserts, respectively, together with macrophages in the lower chambers. Compared with untreated WT control macrophages, 4T1.2 CM- and lactate-activated WT macrophages significantly enhanced the number of migrated cancer cells (FIG. 11B). Gpr132-KO macrophages led to decreased cancer cell migration under all treatment conditions, indicating that Gpr132 deletion in macrophages both attenuated the effects of endogenous lactate from the upper chamber cancer cell and exogenously added 4T1.2 CM and lactate (FIG. 11B). Moreover, Gpr132 deletion in macrophages not only diminished basal breast cancer cell invasion but also completely abrogated breast cancer cell invasion induced by lactate-activated macrophages (FIG. 11C).

To further confirm lactate is a key factor in cancer cell CM that is responsible for M2 macrophage activation to promote cancer cell metastasis, cancer cells were pre-treated with oxamic acid and then used their lactate-depleted CM (FIG. 18D) to culture macrophages used in Boyden Chamber Assays. The results showed that the effects of EO771 or 4T1.2 CM-activated WT macrophages on cancer cell migration and invasion were abolished by oxamic acid pre-treatment (FIG. 23A-B), which was restored by the addition of exogenous lactate in oxamic acid-pretreated cancer cell CM (FIG. 23A-B). Once again, Gpr132-KO macrophages did not respond to these treatments (FIG. 23A-B). Taken together, these data suggest that macrophage activation by cancer cell-derived lactate further promotes breast cancer cell metastasis via Gpr132 in vitro.

### Example 18: Gpr132 Deletion Impedes Breast Cancer Metastasis In Vivo

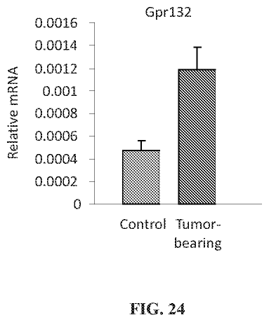

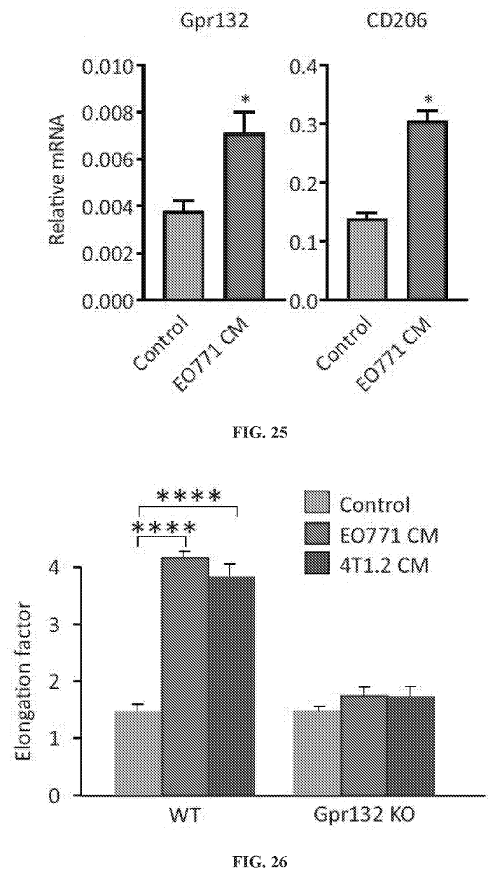

To investigate the in vivo significance of the lactate-Gpr132 axis in breast cancer metastasis, it was first examined if primary tumors could influence Gpr132 expression in pre-metastatic sites such as the lung. The results showed that Gpr132 expression was enhanced in the lung of EO771 tumor-bearing mice compared with tumor-free control mice (FIG. 24). Next, EO771 cells were inoculated into the mammary fat pad of female WT and Gpr132-KO mice, and then spontaneous lung metastasis was examined. Hematoxylin and eosin (H&E) staining showed that the number and size of EO771 lung metastases were significantly decreased in Gpr132-KO mice compared with WT mice (FIG. 12A-C). Recently, a new breast cancer subline, EO771.LMB, has been established, which confers more aggressive lung metastasis without altering primary tumor growth compared with parental EO771 cells. It was found that spontaneous lung metastasis from EO771-LMB cells was also diminished in Gpr132-KO mice compared with WT mice (FIG. 12D-G). In addition to H&E staining-based metastatic foci measurements (FIG. 12D-F), lung metastatic tumor burden has also been quantified using an activatable, pH-responsive fluorescence sensor called Probe 5c that has been demonstrated to “turn on” selectively in tumors but not in normal tissues, thus serving as a tumor indicator. The ratiometric Probe 5c activated much less in the lung metastases of Gpr132-KO mice than those in the lung metastases of WT mice (FIG. 12G), which further suggests that Gpr132 deletion impedes breast cancer metastasis.

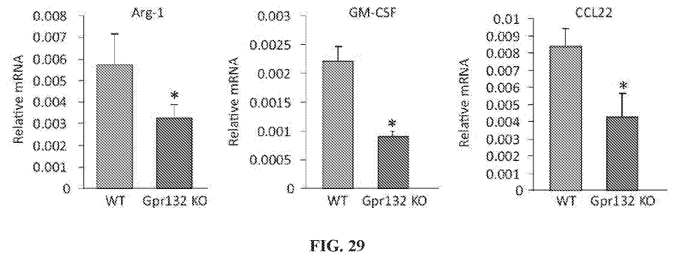

It was next examined whether the reduced lung metastasis in Gpr132-KO mice was related to impaired M2 macrophages. Immunohistochemistry (IHC) and RT-qPCR showed that the expression of M2 macrophage markers, such as CD206, Arg-1, GM-CSF, CCL17, CCL22 and YM-1, was lower in the lung of EO771 tumor-bearing Gpr132-KO mice than in WT mice (FIG. 12H-L and FIG. 29). Together, these data suggest that disruption of the lactate-Gpr132 axis effectively blocks breast cancer metastasis in vivo via compromising M2 macrophage activation.

### Example 19: Gpr132 Correlates with Metastasis and M2 Macrophages in Human Breast Cancer

To assess the clinical significance of Gpr132 in breast cancer metastasis, several datasets were analyzed in PrognoScan. The results revealed that higher Gpr132 expression significantly correlated with lower metastasis-free and relapse-free survival (FIG. 13A). Moreover, linear regression analyses of RNA sequencing data from The Cancer Genome Atlas breast invasive carcinoma database showed that higher Gpr132 expression in breast cancer also significantly correlated with higher expression of M2 macrophage markers, including CD163, CCL17, CCL22, C-C chemokine receptor type 2 (CCR2), toll-like receptor 1 (TLR1), TLR8, transglutaminase 2 (TGM2) and CD200R1 (FIG. 13B). These findings suggest that Gpr132 is clinically associated with breast cancer metastasis and M2 macrophage activation in patients with breast cancer, supporting Gpr132 as a valuable prognostic marker and therapeutic target.

### Example 13: Experimental Procedures

A. Mice

PPARγ flox mice on a C57BL/6 background have been previously described (He et al., 2003). GPR132 knockout mice (Le et al., 2001) on a C57BL/6 background were obtained from the Jackson Laboratory. Mice were fed standard chow ad libitum and kept on a 12-h light, 12-h dark cycle. PPARγ flox mice were bred with Tie2-Cre (Kisanuki et al., 2001) or Lysozyme-Cre (Clausen et al., 1999) transgenic mice to generate mf-g-KO mice. Tie2cre-g-KO was bred with GPR132-KO to obtain mf-g/GPR132 double KO mice. All experiments were conducted using littermates. Sample size estimate was based on power analyses performed using SAS 9.3 TS X64_7PRO platform.

Gpr132-KO mice in a C57BL/6J background were purchased from Jackson Laboratory. For in vivo examination of tumor metastasis, EO771 or EO771-LMB cells (5×10⁵) were injected into the mammary fat pad of 6-8 weeks old female mice. Primary tumors were resected when they reached the indicated volume and then the lungs were harvested at indicated time points for H&E staining to detect spontaneous metastasis as previously described (39). Metastasis of EO771-LMB cells to the lungs was also quantified using a pH-responsive fluorescence probe called 5c, a near infrared PEGylated BODIPY-based dye that activates only in the low pH tumor microenvironment and in proportion to tumor mass (27). Probe 5c in PBS was injected intravenously via retro-orbital route at 0.5 mg/kg dose. Images were acquired from dissected lungs 24 hrs after probe injection using the Cy5.5 filter on an IVIS Lumina imaging system (Caliper Life Sciences). All experiments were conducted using littermates. Sample size estimate was based on power analyses using SAS 9.3 TS X64_7PRO platform. All protocols for mouse experiments were approved by the Institutional Animal Care and Use Committee of UTSW.

B. Orthotopic Fat Pad Injection of Mouse Breast Cancer Cells

The EO771 cell line was derived from a spontaneous mammary tumor in a C57BL/6 mouse (Casey et al., 1951). EO771 cells (2.5×10⁵ or 5×10⁵) were injected into the mammary fat pad of 6-8 weeks old female mice. EO771 cell mixture was prepared with a 1:1 ratio in blank RPMI-1640 medium and matrigel (BD Biosciences). Every 2-3 days, tumor length and width were measured with a caliper and tumor volume was calculated using the formula V=(L×W×W)/2, where V is tumor volume, L is tumor length, and W is tumor width.

C. Immunofluorescence Staining

Tumor tissues were isolated from tumor-bearing mice three weeks after cancer cell injection. Tumors were frozen in OCT compound (Tissue-Tek), cryo-sectioned, and fixed with acetone before staining with antibodies. The tumor sections were blocked with 2% BSA, and then incubated with FITC anti-CD11b antibody (BD Pharmingen; 1:50 dilution) or FITC anti-F4/80 antibody (AbD Serotec; 1:50 dilution). For antibodies without FITC conjugate, the tumor sections were incubated with rat monoclonal anti-endomucin (Santa Cruz Biotechnologies; 1:50 dilution), rabbit monoclonal anti-Ki67 (Cell Signaling; 1:400 dilution), or rabbit polyclonal anti-Phospho-Histone H3 (Ser10) (Cell Signaling; 1:200 dilution). After washing with PBS, the sections were incubated with goat-anti-rat or goat-anti-rabbit IgG-FITC antibody (Santa Cruz Biotechnologies; 1:100 dilutions) for detection. After washing with PBS, cover slips were mounted with the Vectashield medium containing DAPI (Vector Laboratories Burlingame, Calif., USA).

D. Macrophage and Cancer Cell Cultures

For bone marrow-derived macrophage (BMMf) and spleen-derived macrophage

(SpMf), mouse bone marrow or splenocyte were collected with serum-free DMEM (Invitrogen). After passing through a 40 μm cell strainer, the cells were cultured in macrophage differentiation medium (DMEM containing 10% fetal bovine serum (FBS) and 20 ng/ml M-CSF) for 6 days. Rosiglitazone (Cayman Chemical) treatment was conducted at 1 μM. GPR132 overexpression was performed with lentiviral transduction. The luciferase-labeled MDA-MB-231 human breast cancer sub-line (1833) (Kang et al., 2003) was provided by Dr. Joan Massagué (Memorial Sloan-Kettering Cancer Center). The luciferase-labeled 4T1.2 mouse breast cancer subline (Lelekakis et al., 1999) was provided by Drs. Robin Anderson (Peter MacCallum Cancer Centre) and Yibing Kang (Princeton University).

E. Co-Culture of Macrophages and Cancer Cells

Mouse bone marrow and spleen cells were plated in 96-well plate and differentiated into macrophages with 20 ng/ml M-CSF for 9 days. Luciferase-labeled 1833 cells or 4T1.2 cells were then added to the culture dish. At the end point of experiment, cell lysates were collected for luciferase assay to assess cancer cell growth. For the pre-treatment, macrophages were cultured with 1 μM rosiglitazone for the last 24 hrs; the medium was removed and the macrophages were washed before cancer cell seeding.

F. Gene Expression Analyses

Tissue samples were snap frozen in liquid nitrogen and stored at −80° C. RNA was extracted using Trizol (Invitrogen) according to the manufacturer's protocol. RNA was first treated with RNase-free DNase I using the DNA-free kit (Ambion) to remove all genomic DNA, and then reverse-transcribed into cDNA using an ABI High Capacity cDNA RT Kit (Invitrogen). The cDNA was analyzed using real-time quantitative PCR (SYBR Green, Invitrogen) with an Applied Biosystems 7700 Sequence Detection System. Each reaction was performed in triplicate in a 384-well format. The expression of mouse gene was normalized by mouse L19. The expression of human gene was normalized with human GAPDH. Anti-GPR132 antibody (Sigma) was used for western blot detection of GPR132 protein.

G. Immunohistochemistry

Tissue microarrays were purchased from US Biomax, Inc. (Rockville, Md., USA), which contain human normal breast tissues and breast cancer tissues. The immunohistochemistry (IHC) staining was performed as previously described (Su et al., 2014; Zhou et al., 2014). Briefly, after dewaxing with xylence and dehydration with gradient ethanol, the tissue microarrays were incubated with antigen retrieval buffer (BD Biosciences) for 1 hr at 95° C., followed by treatment with 3% hydrogen peroxide (Sigma) for 10 min. Specimens were blocked with 5% defatted milk for 1 hr at room temperature, and incubated with anti-human-GPR132 antibodies (Sigma; 1:100) overnight at 4° C. and then with HRP-conjugated secondary antibodies (1:200) for 30 min at room temperature. Immunostaining was performed using a diaminobenzidine (DAB) kit (Thermo scientific). The expression levels of GPR132 were scored semi-quantitatively according to the staining intensity and distribution using the immunoreactive score as described previously (Su et al., 2014; Zhou et al., 2014). Briefly, the IHC score=staining intensity (negative=0; weak=1; moderate=2; and strong=3)×percentage of positive cells (0%=0; 0-25%=1; 25-50%=3; and 75-100%=4).

Immunohistochemistry (IHC) staining of mouse lung tissues was performed as described (5, 6). Briefly, after dewaxing with xylence and dehydration with gradient ethanol, the sections were incubated with antigen retrieval buffer (BD Biosciences) for 1 hr at 95° C., and then treated with 3% H₂O₂ (Sigma) for 15 min. After blocking with 5% defatted milk for 1 hr at room temperature, specimens were incubated with anti-Gpr132 or anti-CD206 antibodies (1:100) overnight at 4° C., washed and then incubated with HRP-conjugated secondary antibodies (1:200) for 30 min at room temperature. Immunostaining was developed with a diaminobenzidine (DAB) kit (Thermo scientific).

H. TCGA Data Analysis

RNA-Seq and clinical data of breast invasive carcinoma (BRCA) were downloaded from The Cancer Genome Atlas (TCGA) data portal (Cancer Genome Atlas, 2012) and tested for associations. Gene expression for GPR132, CCL2 (MCP-1), MMP9 and PTGS2 (COX-2) were analyzed by linear regression.

I. Statistical Analyses

All statistical analyses were performed with Student's t-Test and represented as mean±standard deviation (SD) unless noted otherwise. For in vivo experiments with >=3 groups, statistical analyses were performed with ANOVA followed by the post hoc Tukey pairwise comparisons. The analysis of TCGA-BRCA RNA seq data for the correlation between Gpr132 and M2 macrophage markers was performed using Pearson's Correlation test. The analysis of the survival data from PrognoScan datasets were performed using Log-rank (Mantel-Cox) test. The p values were designated as *, p<0.05; **, p<0.01; ***, p<0.005; ****, p<0.001; n. s. non-significant (p>0.05).

J. Cell Culture

RAW264.7, EO771, 4T1.2, PY8119, PY230, SCP-4, SCP-6, SCP-28, MDA-1883, MDA-2287 and B16F10 cell lines were cultured in culture media containing 10% FBS (Sigma) and 1:100 antibiotic-antimycotic (Gibco). Conditioned media were obtained from serum-free media after culturing cells for 24 hrs or as indicated. Bone marrow-derived macrophages (BMDMs) and spleen-derived macrophages from WT and Gpr132-KO mice were cultured as previously described. Lactate in the conditioned media was quantified using a Vitros 250 chemistry analyzer at the UT Southwestern Metabolic Phenotyping Core.

K. Lipid Extraction from Cancer Cell Conditional Media

Lipid extraction was performed as described. Briefly, <3 kDa EO771 CM was mixed with methanol and chloroform at a ratio of 1 ml: 1 ml: 2 ml. The mixture was centrifuged at 2000 rpm for 30 min at 4° C. to separate organic and aqueous phases. The organic phase containing extracted lipids was transferred to an empty pre-tared vial and dried under a gentle stream of N2. Dried extracts were dissolved in DMSO and used for subsequent experiments.

L. Adherence Assay

Cancer cells were treated with the conditioned medium from indicated macrophage for 2 hrs. Adherence of treated breast cancer cells to fibronectin was evaluated as described.

M. Boyden Chamber Assay

Migration or invasion of breast cancer cells was examined using 24-well Boyden chambers (Corning, Corning, N.Y.) with transwell inserts of 8 μM pore size, coated without (for migration) or with matrigel (for invasion) as previously reported. Macrophages with indicated treatments were seeded in the lower chambers, and breast cancer cells (10⁵ cells/well) were plated on the inserts and cultured in the upper chambers at 37° C. for 6 hrs (migration) or 24 hrs (invasion). The migrated or invaded cells were fixed with methanol and stained with crystal violet (0.05%, sigma), and then counted as cells per field of view under microscope or measured as OD570 after crystal violet was dissolved.

N. Flow Cytometry

After blocking with 10% BSA for 10 min, cells were incubated with anti-CD206 (Rabbit polyclonal, Abcam) or anti-Gpr132 (Rabbit polyclonal, Sigma) antibodies (1:100) for 30 min at 4° C., washed with cold PBS, and then incubated with FITC-conjugated secondary antibodies (1:100) for 30 min at 4° C. After washing twice using cold PBS, cells were analyzed by flow cytometer (Becton Dickinson).

O. RNA Isolation and Real-Time RT-qPCR

RNA was isolated from cells or mouse tissues using Trizol (Invitrogen) following the manufacturer's instructions. RNA (200-500 ng) was first treated with RNase-free DNase I using the DNA-free kit (Ambion) to remove all genomic DNA, and then reverse-transcribed into cDNA using an ABI High Capacity cDNA RT Kit (Invitrogen). The cDNA was analyzed using real-time quantitative PCR (SYBR Green, Invitrogen) with an Applied Biosystems 7900 Sequence Detection System. Each reaction was performed in triplicate in a 384-well format. The expression of each gene was normalized by that of mouse ribosomal protein L19.

P. Western Blot

After receiving indicated treatments, macrophages were harvested for protein extraction. Protein concentration was examined by a BCA assay (Thermo Scientific).

Samples of 20 μg protein were applied to SDS polyacrylamide gels. After protein transfer, membranes were incubated with antibodies for Arg-1 (rabbit polyclonal, Santa Cruz Biotechnology), CD206, Gpr132, or β-actin (mouse monoclonal, Sigma) (1:1000) overnight at 4° C. Membranes were washed, and then incubated with HRP-conjugated secondary antibodies (1:1000) for 1 hr at room temperature before signal detection by chemiluminescence (Pierce). Densitometric quantification was performed by Image-Pro Plus 6.0 software (Media Cybernetics). PathScan Akt Signaling Antibody Array (Cell Signaling) was incubated with macrophage proteins and analyzed following kit instructions.

Q. Immunofluorescence

Macrophage cultures grown on cover slips were washed in PBS and fixed with acetone. After blocking with 5% BSA, cells were incubated with FITC anti-CD11b antibody (BD Pharmingen; 1:50) for 2 hrs at room temperature, and then with DAPI (Vector Laboratories Burlingame, Calif., USA). After washing with PBS, cover slips were mounted using 80% glycerol.

R. Co-Immunoprecipitation and Liquid Chromatography Mass Spectrometry (LC-MS)

WT or Gpr132-KO BMDM lysate was incubated with concentrated <3 kDa EO771 CM for 4 hrs. This mixture of 1 ml volume was then incubated with 10 μl anti-Gpr132 antibody and 40 μl protein A/G plus-agarose beads overnight at 4° C. After the beads were washed, the IP eluent was collected for lactate quantification. Lactate was extracted and analyzed as reported (7). Briefly, 400 μl of cold MeOH was added to 100 μl of the IP eluent. Samples were mixed and then stored at −80° C. overnight. The next morning, samples were spun at 14,000 g for 10 min, and the supernatant was collected. After drying the supernatant via speedvac, the extract was dissolved in H₂O (40 μl) and a portion of this sample (10 μl) analyzed by LC-MS. Lactate was measured using a TSQ Quantiva LC-MS instrument fitted with a Luna NH2 HPLC column (3.5 m, 4.6 mm×100 mm, Phenomenex). The following LC solvents were used: buffer A, 95:5 H₂O/ACN, 20 mM ammonium hydroxide, 20 mM ammonium acetate; buffer B, 100% ACN. A typical LC run was 23 min long with a flow rate of 0.4 ml/min and consisted of the following steps: 85 to 30% buffer B over 3 min, 30 to 2% buffer B over 9 min, 2% buffer B for 3 min, 2% to 85% buffer B over 1 min, and 85% buffer B for 7 min. MS analysis was performed using electrospray ionization (ESI) in negative ion mode with the following source parameters: spray voltage 3.5 kV, ion transfer tube temperature of 325° C., and a vaporizer temperature of 275° C. SRM [collision energy (CE) of 11 V, RF lens set at 32 V] was used to detect lactate (m/z 89.3→43.2). Relative abundance of lactate was determined by measuring the lactate peak area.

S. Breast Cancer Patient Data Analysis

RNA-Seq data from breast invasive carcinoma (BRCA) were downloaded from The Cancer Genome Atlas (TCGA) data portal on Jul. 20, 2016 and tested for associations. Gene expression for GPR132, CD163, CCL17, CCL22, CCR2, TLR1, TLR8, TGM2 and CD200R1 were analyzed by linear regression. Datasets were downloaded from PrognoScan, a database for meta-analysis of the prognostic value of genes, and used to plot survival curves.

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

### REFERENCES

The following references, to the extent that they provide exemplary procedural or other details supplementary to those set forth herein, are specifically incorporated herein by reference.
Ahmadian, M., Suh, J. M., Hah, N., Liddle, C., Atkins, A. R., Downes, M., and Evans, R. M. (2013). PPARgamma signaling and metabolism: the good, the bad and the future. Nature medicine 19, 557-566.
Aldred M A, M. C., Gimm O, Hoang-Vu C, Krause U, Dralle H, Jhiang S, Eng C (2003). Peroxisome proliferator-activated receptor gamma is frequently downregulated in a diversity of sporadic nonmedullary thyroid carcinomas. Oncogene 22, 3412-3416.
Apostoli, A. J., Roche, J. M., Schneider, M. M., SenGupta, S. K., Di Lena, M. A., Rubino, R. E., Peterson, N. T., and Nicol, C. J. (2015). Opposing roles for mammary epithelial-specific PPARgamma signaling and activation during breast tumour progression. Molecular cancer 14, 85.
Acharyya S, et al. (2012) A CXCL1 paracrine network links cancer chemoresistance and metastasis. Cell 150(1):165-178.
Ahmed K, et al. (2010) An autocrine lactate loop mediates insulin-dependent inhibition of lipolysis through GPR81. Cell metabolism 11(4):311-319.
Barone, B. B., Yeh, H. C., Snyder, C. F., Peairs, K. S., Stein, K. B., Derr, R. L., Wolff, A. C., and Brancati, F. L. (2010). Postoperative mortality in cancer patients with preexisting diabetes: systematic review and meta-analysis. Diabetes care 33, 931-939.
Bingle L, B. N., Lewis CE (2002). The role of tumour-associated macrophages in tumour progression: implications for new anticancer therapies. The Journal of Pathology 196, 254-265.
Blaser, M. J., Chyou, P. H., and Nomura, A. (1995). Age at establishment of Helicobacter pylori infection and gastric carcinoma, gastric ulcer, and duodenal ulcer risk. Cancer research 55, 562-565.
Bolick D T, et al. (2009) G2A deficiency in mice promotes macrophage activation and atherosclerosis. Circulation research 104(3):318-327.
Bosetti, C., Rosato, V., Buniato, D., Zambon, A., La Vecchia, C., and Corrao, G. (2013). Cancer risk for patients using thiazolidinediones for type 2 diabetes: a meta-analysis. The oncologist 18, 148-156.
Burstein, H. J., Demetri, G. D., Mueller, E., Sarraf, P., Spiegelman, B. M., and Winer, E. P. (2003). Use of the peroxisome proliferator-activated receptor (PPAR) gamma ligand troglitazone as treatment for refractory breast cancer: a phase II study. Breast cancer research and treatment 79, 391-397.
Byles V, et al. (2013) The TSC-mTOR pathway regulates macrophage polarization. Nature communications 4:2834.
Cancer Genome Atlas, N. (2012). Comprehensive molecular portraits of human breast tumours. Nature 490, 61-70.
Casey, A. E., Laster, W. R., Jr., and Ross, G. L. (1951). Sustained enhanced growth of carcinoma EO771 in C57 black mice. Proceedings of the Society for Experimental Biology and Medicine. Society for Experimental Biology and Medicine 77, 358-362.
Chen J, et al. (2011) CCL18 from tumor-associated macrophages promotes breast cancer metastasis via PITPNM3. Cancer cell 19(4):541-555.
Chen P & Bonaldo P (2013) Role of macrophage polarization in tumor angiogenesis and vessel normalization: implications for new anticancer therapies. International review of cell and molecular biology 301:1-35.
Chen P, et al. (2015) Collagen VI regulates peripheral nerve regeneration by modulating macrophage recruitment and polarization. Acta neuropathologica 129(1):97-113.
Clausen, B. E., Burkhardt, C., Reith, W., Renkawitz, R., and Forster, I. (1999). Conditional gene targeting in macrophages and granulocytes using LysMcre mice. Transgenic Res 8, 265-277.
Colegio O R, et al. (2014) Functional polarization of tumour-associated macrophages by tumour-derived lactic acid. Nature 513(7519):559-563.
Condeelis J & Pollard J W (2006) Macrophages: obligate partners for tumor cell migration, invasion, and metastasis. Cell 124(2):263-266.
Coussens, L. M., and Werb, Z. (2002). Inflammation and cancer. Nature 420, 860-867.
Covarrubias A J, et al. (2016) Akt-mTORC1 signaling regulates Acly to integrate metabolic input to control of macrophage activation. eLife 5.
Drzewoski, J., Drozdowska, A., and Sliwinska, A. (2011). Do we have enough data to confirm the link between antidiabetic drug use and cancer development? Polskie Archiwum Medycyny Wewnetrznej 121, 81-87.
Feng, Y. H., Velazquez-Torres, G., Gully, C., Chen, J., Lee, M. H., and Yeung, S. C. (2011). The impact of type 2 diabetes and antidiabetic drugs on cancer cell growth. Journal of cellular and molecular medicine 15, 825-836.
Fenner, M. H., and Elstner, E. (2005). Peroxisome proliferator-activated receptor-gamma ligands for the treatment of breast cancer. Expert opinion on investigational drugs 14, 557-568.
Frohlich, E., and Wahl, R. (2015). Chemotherapy and chemoprevention by thiazolidinediones. BioMed research international 2015, 845340.
Goetze, S., Bungenstock, A., Czupalla, C., Eilers, F., Stawowy, P., Kintscher, U., Spencer-Hansch, C., Graf, K., Nurnberg, B., Law, R. E., et al. (2002). Leptin induces endothelial cell migration through Akt, which is inhibited by PPARgamma-ligands. Hypertension 40, 748-754.
Gordon S (2003) Alternative activation of macrophages. Nature reviews. Immunology 3(1):23-35.
He, W., Barak, Y., Hevener, A., Olson, P., Liao, D., Le, J., Nelson, M., Ong, E., Olefsky, J. M., and Evans, R. M. (2003). Adipose-specific peroxisome proliferator-activated receptor gamma knockout causes insulin resistance in fat and liver but not in muscle. Proc Natl Acad Sci USA 100, 15712-15717.
Hevener, A. L., Olefsky, J. M., Reichart, D., Nguyen, M. T., Bandyopadyhay, G., Leung, H. Y., Watt, M. J., Benner, C., Febbraio, M. A., Nguyen, A. K., et al. (2007). Macrophage PPAR gamma is required for normal skeletal muscle and hepatic insulin sensitivity and full antidiabetic effects of thiazolidinediones. The Journal of clinical investigation 117, 1658-1669.
Johnstone C N, et al. (2015) Functional and molecular characterisation of EO771.LMB tumours, a new C57BL/6-mouse-derived model of spontaneously metastatic mammary cancer. Disease models & mechanisms 8(3):237-251.
Jones S E (2008) Metastatic breast cancer: the treatment challenge. Clinical breast cancer 8(3):224-233.
Justus C R, Dong L, & Yang L V (2013) Acidic tumor microenvironment and pH-sensing G protein-coupled receptors. Frontiers in physiology 4:354.
Kabarowski, J. H. (2009). G2A and LPC: regulatory functions in immunity. Prostaglandins & other lipid mediators 89, 73-81.
Kang, Y., Siegel, P. M., Shu, W., Drobnjak, M., Kakonen, S. M., Cordon-Cardo, C., Guise, T. A., and Massague, J. (2003). A multigenic program mediating breast cancer metastasis to bone. Cancer Cell 3, 537-549.
Keshamouni, V. G., Arenberg, D. A., Reddy, R. C., Newstead, M. J., Anthwal, S., and Standiford, T. J. (2005). PPAR-gamma activation inhibits angiogenesis by blocking ELR+CXC chemokine production in non-small cell lung cancer. Neoplasia 7, 294-301.
Khamis Z I, Sahab Z J, & Sang Q X (2012) Active roles of tumor stroma in breast cancer metastasis. International journal of breast cancer 2012:574025.
Kisanuki, Y. Y., Hammer, R. E., Miyazaki, J., Williams, S. C., Richardson, J. A., and Yanagisawa, M. (2001). Tie2-Cre transgenic mice: a new model for endothelial cell-lineage analysis in vivo. Dev Biol 230, 230-242.
Komohara, Y., Jinushi, M., and Takeya, M. (2014). Clinical significance of macrophage heterogeneity in human malignant tumors. Cancer science 105, 1-8.
Krzeszinski, J. Y., and Wan, Y. (2015). New therapeutic targets for cancer bone metastasis. Trends in pharmacological sciences 36, 360-373.
Kumar, A. P., Quake, A. L., Chang, M. K., Zhou, T., Lim, K. S., Singh, R., Hewitt, R. E., Salto-Tellez, M., Pervaiz, S., and Clement, M. V. (2009). Repression of NHE1 expression by PPARgamma activation is a potential new approach for specific inhibition of the growth of tumor cells in vitro and in vivo. Cancer research 69, 8636-8644.
Kuper, H., Adami, H. O., and Trichopoulos, D. (2000). Infections as a major preventable cause of human cancer. J Intern Med 248, 171-183.
Larson, S. D., Jackson, L. N., Chen, L. A., Rychahou, P. G., and Evers, B. M. (2007). Effectiveness of siRNA uptake in target tissues by various delivery methods. Surgery 142, 262-269.
Le, L. Q., Kabarowski, J. H., Weng, Z., Satterthwaite, A. B., Harvill, E. T., Jensen, E. R., Miller, J. F., and Witte, O. N. (2001). Mice lacking the orphan G protein-coupled receptor G2A develop a late-onset autoimmune syndrome. Immunity 14, 561-571.
Lefterova, M. I., Haakonsson, A. K., Lazar, M. A., and Mandrup, S. (2014). PPARgamma and the global map of adipogenesis and beyond. Trends in endocrinology and metabolism: TEM 25, 293-302.
Lelekakis, M., Moseley, J. M., Martin, T. J., Hards, D., Williams, E., Ho, P., Lowen, D., Javni, J., Miller, F. R., Slavin, J., et al. (1999). A novel orthotopic model of breast cancer metastasis to bone. Clinical & experimental metastasis 17, 163-170.
Lin, E. Y., Nguyen, A. V., Russell, R. G., and Pollard, J. W. (2001). Colony-stimulating factor 1 promotes progression of mammary tumors to malignancy. The Journal of experimental medicine 193, 727-740.
Liu C, et al. (2009) Lactate inhibits lipolysis in fat cells through activation of an orphan G-protein-coupled receptor, GPR81. The Journal of biological chemistry 284(5):2811-2822.
Mao Y, Keller E T, Garfield D H, Shen K, & Wang J (2013) Stromal cells in tumor microenvironment and breast cancer. Cancer metastasis reviews 32(1-2):303-315.
Mizuno H, Kitada K, Nakai K, & Sarai A (2009) PrognoScan: a new database for meta-analysis of the prognostic value of genes. BMC medical genomics 2:18.
Monami, M., Dicembrini, I., and Mannucci, E. (2014). Thiazolidinediones and cancer: results of a meta-analysis of randomized clinical trials. Acta diabetologica 51, 91-101.
Mueller, E., Smith, M., Sarraf, P., Kroll, T., Aiyer, A., Kaufman, D. S., Oh, W., Demetri, G., Figg, W. D., Zhou, X. P., et al. (2000). Effects of ligand activation of peroxisome proliferator-activated receptor gamma in human prostate cancer. Proceedings of the National Academy of Sciences of the United States of America 97, 10990-10995.
Mueller E, S. P., Tontonoz P, Evans R M, Martin K J, Zhang M, Fletcher C, Singer S, Spiegelman B M (1998). Terminal differentiation of human breast cancer through PPAR gamma. Mol Cell 1, 465-470.
Murakami N, Yokomizo T, Okuno T, & Shimizu T (2004) G2A is a proton-sensing G-protein-coupled receptor antagonized by lysophosphatidylcholine. The Journal of biological chemistry 279(41):42484-42491.
Noy, R., and Pollard, J. W. (2014). Tumor-associated macrophages: from mechanisms to therapy. Immunity 41, 49-61.
Obinata H, Hattori T, Nakane S, Tatei K, & Izumi T (2005) Identification of 9-hydroxyoctadecadienoic acid and other oxidized free fatty acids as ligands of the G protein-coupled receptor G2A. The Journal of biological chemistry 280(49):40676-40683.
Park, J., Morley, T. S., Kim, M., Clegg, D. J., and Scherer, P. E. (2014). Obesity and cancer--mechanisms underlying tumour progression and recurrence. Nature reviews. Endocrinology 10, 455-465.
Parks B W, Srivastava R, Yu S, & Kabarowski J H (2009) ApoE-dependent modulation of HDL and atherosclerosis by G2A in LDL receptor-deficient mice independent of bone marrow-derived cells. Arteriosclerosis, thrombosis, and vascular biology 29(4):539-547.
Pollard J W (2004) Tumour-educated macrophages promote tumour progression and metastasis. Nature reviews. Cancer 4(1):71-78.
Pyonteck S M, et al. (2013) CSF-1R inhibition alters macrophage polarization and blocks glioma progression. Nature medicine 19(10):1264-1272.
Qian, B. Z., and Pollard, J. W. (2010). Macrophage diversity enhances tumor progression and metastasis. Cell 141, 39-51.
Quail D F & Joyce J A (2013) Microenvironmental regulation of tumor progression and metastasis. Nature medicine 19(11):1423-1437.
Radu, C. G., Yang, L. V., Riedinger, M., Au, M., and Witte, O. N. (2004). T cell chemotaxis to lysophosphatidylcholine through the G2A receptor. Proceedings of the National Academy of Sciences of the United States of America 101, 245-250.
Richardson, L. C., and Pollack, L. A. (2005). Therapy insight: Influence of type 2 diabetes on the development, treatment and outcomes of cancer. Nature clinical practice. Oncology 2, 48-53.
Roland C L, et al. (2014) Cell surface lactate receptor GPR81 is crucial for cancer cell survival. Cancer research 74(18):5301-5310.
Ruffell, B., Affara, N. I., and Coussens, L. M. (2012). Differential macrophage programming in the tumor microenvironment. Trends in immunology 33, 119-126.
Ruffell, B., and Coussens, L. M. (2015). Macrophages and therapeutic resistance in cancer. Cancer Cell 27, 462-472.
Saez, E., Rosenfeld, J., Livolsi, A., Olson, P., Lombardo, E., Nelson, M., Banayo, E., Cardiff, R. D., Izpisua-Belmonte, J. C., and Evans, R. M. (2004). PPAR gamma signaling exacerbates mammary gland tumor development. Genes & development 18, 528-540.
Saez, E., Tontonoz, P., Nelson, M. C., Alvarez, J. G., Ming, U. T., Baird, S. M., Thomazy, V. A., and Evans, R. M. (1998). Activators of the nuclear receptor PPARgamma enhance colon polyp formation. Nature medicine 4, 1058-1061.
Sarraf P, M. E., Smith W M, Wright H M, Kum J B, Aaltonen L A, de la Chapelle A, Spiegelman B M, Eng C (1999). Loss-of-function mutations in PPAR gamma associated with human colon cancer. Mol Cell 3, 799-804.
Scholl, S. M., Pallud, C., Beuvon, F., Hacene, K., Stanley, E. R., Rohrschneider, L., Tang, R., Pouillart, P., and Lidereau, R. (1994). Anti-colony-stimulating factor-1 antibody staining in primary breast adenocarcinomas correlates with marked inflammatory cell infiltrates and prognosis. Journal of the National Cancer Institute 86, 120-126.
Scoditti, E., Massaro, M., Carluccio, M. A., Distante, A., Storelli, C., and De Caterina, R. (2010). PPARgamma agonists inhibit angiogenesis by suppressing PKCalpha- and CREB-mediated COX-2 expression in the human endothelium. Cardiovascular research 86, 302-310.
Shacter, E., and Weitzman, S. A. (2002). Chronic inflammation and cancer. Oncology (Williston Park) 16, 217-226, 229; discussion 230-212.
Simpson-Haidaris P J & Rybarczyk B (2001) Tumors and fibrinogen. The role of fibrinogen as an extracellular matrix protein. Annals of the New York Academy of Sciences 936:406-425.
Skelhorne-Gross, G., Reid, A. L., Apostoli, A. J., Di Lena, M. A., Rubino, R. E., Peterson, N. T., Schneider, M., SenGupta, S. K., Gonzalez, F. J., and Nicol, C. J. (2012). Stromal adipocyte PPARgamma protects against breast tumorigenesis. Carcinogenesis 33, 1412-1420.
Smith, U., and Gale, E. A. (2009). Does diabetes therapy influence the risk of cancer? Diabetologia 52, 1699-1708.
Smith, U., and Gale, E. A. (2010). Cancer and diabetes: are we ready for prime time? Diabetologia 53, 1541-1544.
Sousa S, et al. (2015) Human breast cancer cells educate macrophages toward the M2 activation status. Breast cancer research: BCR 17:101.
Su, S., Liu, Q., Chen, J., Chen, J., Chen, F., He, C., Huang, D., Wu, W., Lin, L., Huang, W., et al. (2014). A positive feedback loop between mesenchymal-like cancer cells and macrophages is essential to breast cancer metastasis. Cancer Cell 25, 605-620.
Tontonoz P, S. S., Forman B M, Sarraf P, Fletcher J A, Fletcher C D, Brun R P, Mueller E, Altiok S, Oppenheim H, Evans R M, Spiegelman B M (1997). Terminal differentiation of human liposarcoma cells induced by ligands for peroxisome proliferator-activated receptor gamma and the retinoid X receptor. Proc Natl Acad Sci USA 94, 237-241.
Tsujikawa T, et al. (2013) Autocrine and paracrine loops between cancer cells and macrophages promote lymph node metastasis via CCR4/CCL22 in head and neck squamous cell carcinoma. International journal of cancer 132(12):2755-2766.
Uray, I. P., Rodenberg, J. M., Bissonnette, R. P., Brown, P. H., and Mancini, M. A. (2012). Cancer-preventive rexinoid modulates neutral lipid contents of mammary epithelial cells through a peroxisome proliferator-activated receptor gamma-dependent mechanism. Molecular pharmacology 81, 228-238.
Vangaveti V, Baune B T, & Kennedy R L (2010) Hydroxyoctadecadienoic acids: novel regulators of macrophage differentiation and atherogenesis. Therapeutic advances in endocrinology and metabolism 1(2):51-60.
Wan, Y., Chong, L. W., and Evans, R. M. (2007a). PPAR-gamma regulates osteoclastogenesis in mice. Nat Med 13, 1496-1503.
Wan, Y., Saghatelian, A., Chong, L. W., Zhang, C. L., Cravatt, B. F., and Evans, R. M. (2007b). Maternal PPAR gamma protects nursing neonates by suppressing the production of inflammatory milk. Genes & development 21, 1895-1908.
Wang C, et al. (2013) Characterization of murine macrophages from bone marrow, spleen and peritoneum. BMC immunology 14:6.
Wei W, et al. (2016) Ligand Activation of ERRalpha by Cholesterol Mediates Statin and Bisphosphonate Effects. Cell metabolism 23(3):479-491.
Welch, J. S., Ricote, M., Akiyama, T. E., Gonzalez, F. J., and Glass, C. K. (2003). PPARgamma and PPARdelta negatively regulate specific subsets of lipopolysaccharide and IFN-gamma target genes in macrophages. Proceedings of the National Academy of Sciences of the United States of America 100, 6712-6717.
Weng, Z., Fluckiger, A. C., Nisitani, S., Wahl, M. I., Le, L. Q., Hunter, C. A., Fernal, A. A., Le Beau, M. M., and Witte, O. N. (1998). A DNA damage and stress inducible G protein-coupled receptor blocks cells in G2/M. Proceedings of the National Academy of Sciences of the United States of America 95, 12334-12339.
Williams C B, Yeh E S, & Soloff A C (2016) Tumor-associated macrophages: unwitting accomplices in breast cancer malignancy. NPJ breast cancer 2.
Wyckoff J, et al. (2004) A paracrine loop between tumor cells and macrophages is required for tumor cell migration in mammary tumors. Cancer research 64(19):7022-7029.
Xiong H, et al. (2016) Activatable Water-Soluble Probes Enhance Tumor Imaging by Responding to Dysregulated pH and Exhibiting High Tumor-to-Liver Fluorescence Emission Contrast. Bioconjugate chemistry 27(7):1737-1744.
Yang, L. V., Radu, C. G., Wang, L., Riedinger, M., and Witte, O. N. (2005). Gi-independent macrophage chemotaxis to lysophosphatidylcholine via the immunoregulatory GPCR G2A. Blood 105, 1127-1134.
Yuan M, Breitkopf S B, Yang X, & Asara J M (2012) A positive/negative ion-switching, targeted mass spectrometry-based metabolomics platform for bodily fluids, cells, and fresh and fixed tissue. Nature protocols 7(5):872-881.
Yumimoto K, et al. (2015) F-box protein FBXW7 inhibits cancer metastasis in a non-cell-autonomous manner. The Journal of clinical investigation 125(2):621-635.
Zhang, Q. W., Liu, L., Gong, C. Y., Shi, H. S., Zeng, Y. H., Wang, X. Z., Zhao, Y. W., and Wei, Y. Q. (2012). Prognostic significance of tumor-associated macrophages in solid tumor: a meta-analysis of the literature. PloS one 7, e50946.
Zhou, W., Fong, M. Y., Min, Y., Somlo, G., Liu, L., Palomares, M. R., Yu, Y., Chow, A., O'Connor, S. T., Chin, A. R., et al. (2014). Cancer-secreted miR-105 destroys vascular endothelial barriers to promote metastasis. Cancer Cell 25, 501-515.

1. A method for reducing and/or inhibiting cancer cell proliferation, tumor growth, and/or metastasis in a subject having cancerous cells, tumor growth and/or metastasis, the method comprising administering an effective amount of an agent that inhibits GPR132 to the subject.

2. A method for treating an inflammatory disorder in a subject in need thereof comprising administering an effective amount of an agent that inhibits GPR132 to the subject.

3. The method of claim 2, wherein the inflammatory disorder is associated with a cancerous or autoimmune condition.

4. The method of claim 1, wherein the tumor or cancer cell has no detectable expression of GPR132 or wherein the tumor or cancer cell has a level of GPR132 expression that is substantially the same or less than the level of expression in a non-cancerous cell control and wherein the tumor or cancer cell and the control cell are the same cell type.

5. The method of any one of claims 1-4, wherein GPR132 is inhibited in a macrophage.

6. The method of any one of claims 1-5, wherein the agent is a nucleic acid inhibitor, an antibody inhibitor, a peptide or polypeptide inhibitor, or a small molecule inhibitor.

7. The method of any one of claims 1-6, wherein the agent is administered systemically, parenterally, intravenously, or intratumorally.

8. The method of any one of claims 1-7, wherein the tumor is comprised within a microenvironment and wherein the tumor microenvironment comprises macrophages expressing GPR132.

9. The method of any one of claim 1, or 3-8, wherein the cancer cell or tumor is a breast cancer cell or a tumor in the breast.

10. The method of any one of claims 1-9, wherein the subject is a human subject that has been diagnosed with a cancer or inflammatory disorder or has previously been treated for the cancer or inflammatory disorder.

11. A method for classifying a cancer patient, comprising:
measuring the level of expression of GPR132 in a cancer sample of the patient; and
classifying the patient as having a favorable prognosis based on a lower expression of GPR132 in the sample as compared to a control or reference level that is normal or indicating favorable prognosis, or classifying the patient as having a poor prognosis based on a higher expression level of GPR132 as compared to the control or a reference level.

12. A method for treating a patient for aggressive or non-aggressive cancer comprising:
treating the patient for aggressive cancer after the patient is determined to have an elevated level of GPR132 expression in a biological sample from the patient compared to a biological sample from a patient with non-aggressive cancer; or
treating the patient for non-aggressive cancer after the patient is determined to have a GPR132 level of expression that is lower than or not significantly different than a level of GPR132 expression in a biological sample from a patient with non-aggressive cancer.

13. The method of claim 12, wherein the cancer comprises breast cancer.

14. The method of claim 12 or 13, wherein the biological sample comprises a hematopoietic cell or a cell progeny thereof, wherein the biological sample comprises macrophages, and/or wherein the biological sample excludes tumor cells.

15. The method of any one of claims 12-14, wherein the treatment for non-aggressive cancer comprises surgical incision of the primary tumor and/or excludes chemotherapy.

16. The method of any one of claims 12-15, wherein the treatment for the aggressive cancer comprises a GRP132 inhibitor and/or rosiglitazone.

17. A kit comprising an agent for detecting GPR132 expression.

18. A method for diagnosing a patient with aggressive or non-aggressive cancer comprising:
diagnosing the patient as having or likely to have aggressive cancer or providing an analysis or report that the patient has or likely has aggressive cancer when the expression or activity level of GPR132 in a biological sample from the patient is determined to be elevated compared to the expression or activity level of GPR132 in a biological sample from a patient with non-aggressive cancer; or
diagnosing the patient as having or likely to have non-aggressive cancer or providing an analysis or report that the patient has or likely has non-aggressive cancer when the expression or activity level of GPR132 in the biological sample from the patient is determined to be not significantly different or lower than the expression or activity level of GPR132 in a biological sample from a patient with non-aggressive cancer.

19. The method of claim 18, wherein the method further comprises measuring the expression or activity level of GPR132 in a biological sample from the patient; wherein the biological sample comprises macrophages or hematopoietic cells or a cell progeny thereof.

20. The method of claim 18 or 19, wherein the biological sample excludes tumor cells.

21. The method of any one of claims 18-20, wherein the method further comprises treating the patient for aggressive or non-aggressive cancer, wherein the treatment for non-aggressive cancer comprises surgical incision of the primary tumor, wherein the treatment for non-aggressive cancer excludes chemotherapy, and/or wherein the treatment for the aggressive cancer comprises a GRP132 inhibitor and/or rosiglitazone.
